(19) United States
(12) Patent Application Publication
(10) Pub. No.: US 2021/0369147 A1
(43) Pub. Date: Dec. 2, 2021

(54) Multimodal Neuroimaging-Based Diagnostic Systems and Methods for Detecting Tinnitus

(71) Applicant: The Regents of the University of California, Oakland, CA (US)

(72) Inventors: Steven Wan Cheung, San Francisco, CA (US); Srikantan Nagarajan, San Francisco, CA (US); Leighton B. Hinkley, San Francisco, CA (US)

(21) Appl. No.: 17/258,403

(22) PCT Filed: Jul. 17, 2019
(86) PCT No.: PCT/US2019/042219

§ 371 Date: Jan. 6, 2021

Related U.S. Application Data
(60) Provisional application No. 62/700,129, filed on Jul. 18, 2018.

(51) Int. Cl. A61B 5/12; A61B 5/245; A61B 5/00; G01R 33/48

(52) U.S. Cl. CPC A61B 5/128; G01R 33/4806; A61B 5/0042; A61B 5/245

(57) ABSTRACT

The present disclosure includes provides methods for assessing resting-state fMRI functional connectivity, resting-state MEGI functional connectivity, and/or task-based spatiotemporal auditory cortical activity latency in a subject to detect, monitor, and/or diagnose Tinnitus, with or without hearing impairment. The present disclosure also provides systems, devices, and methods for diagnosing Tinnitus and/or hearing impairment in a subject. Also provided are systems configured for performing the disclosed methods and computer readable medium storing instructions for performing steps of the disclosed methods.

### CROSS-REFERENCE

This application claims the benefit of U.S. Provisional Patent Application No. 62/700,129, filed Jul. 18, 2018, which application is incorporated herein by reference in its entirety.

### INTRODUCTION

The present disclosure provides multimodal neuroimaging-based systems, devices, and methods for assessing brain activity and synchrony using functional magnetic resonance imaging (fMRI) and magnetoencephalographic imaging (MEGI). More specifically, present disclosure relates detection and/or monitoring of Tinnitus in an individual.

Tinnitus (e.g. subjective Tinnitus) is a disorder of phantom auditory percepts in the absence of physical sound stimuli. Non-observable symptoms include ringing, hissing, buzzing, roaring, and the like that are reported to emanate from one ear, both ears, or somewhere in the head. Occupational noise exposure is one reason for the onset of constant, chronic Tinnitus. Military personnel, Veterans, and civilians in certain professions, such as firefighters and construction workers, are at increased risk for persistent auditory phantoms triggered by hearing loss. With widespread access to consumer electronics, growing affinity for portable music appliances worldwide may contribute to increased hearing loss and Tinnitus.

Tinnitus diagnosis and severity are dependent on subjective self-report survey instruments and corroborative medical evidence. An objective tool to detect Tinnitus and monitor treatment response would allow for understanding the biological basis of Tinnitus and advance care for patients with increased risk for persistent auditory phantoms triggered by hearing loss or other known causes. The systems and methods disclose herein provide a diagnostic tool anchored on multimodal neuroimaging-based objective measurements that would be applicable across a wide range of hearing loss profiles to detect and monitor Tinnitus.

### SUMMARY

The present disclosure includes provides methods for assessing resting-state fMRI functional connectivity (RS-fMRI), resting-state MEGI (RS-MEGI) functional connectivity, and/or task-based spatiotemporal auditory cortical activity estimated from MEGI in an individual subject to detect, monitor, and/or diagnose Tinnitus with or without hearing impairment. The present disclosure also provides systems, devices, and methods for diagnosing and/or monitoring Tinnitus and/or hearing impairment in a subject. Also provided are systems configured for performing the disclosed methods and computer readable medium storing instructions for performing steps of the disclosed methods.

Aspects of the present disclosure include a non-transitory computer readable medium storing instructions that, when executed by a computing device, cause the computing device to perform the steps for detecting and/or monitoring Tinnitus, as provided herein.

In one aspect, the present disclosure relates to a method of detecting Tinnitus in a subject. The method comprising acquiring functional magnetic resonance imaging (fMRI) functional connectivity data or magnetoencephalographic imaging (MEGI) functional connectivity data of at least one of the caudate nucleus, the caudate head, the caudate body, the frontal lobe, and the auditory cortex regions of the brain of the subject; assessing the fMRI functional connectivity data or the MEGI functional connectivity data in at the at least one region of the brain; determining if the fMRI functional connectivity data or the MEGI functional connectivity data are above, below, or at a reference level associated with at least one or more pathology profiles of Tinnitus, wherein at least one pathology profile of Tinnitus comprises: modulated fMRI functional connectivity between the caudate nucleus and the rest of the brain as compared to the reference level; modulated MEGI functional connectivity in the frontal lobe as compared to the reference level; or modulated MEGI functional connectivity in the auditory cortex regions as compared to the reference level. In some embodiments, the modulated fMRI functional connectivity comprises increased fMRI functional connectivity between the caudate nucleus and the auditory cortex region of the brain. In some embodiments, the modulated fMRI functional connectivity comprises decreased fMRI functional connectivity between the caudate nucleus and the frontal lobe region of the brain. In some embodiments, the modulated MEGI functional connectivity comprises increased MEGI functional connectivity in the frontal cortex of the frontal lobe region of the brain. In some embodiments, the modulated MEGI functional connectivity comprises increased MEGI functional connectivity in the auditory cortex of the temporal lobe region of the brain. In some embodiments, the modulated MEGI functional connectivity comprises decreased MEGI functional connectivity in the auditory cortex of the temporal lobe region of the brain. In some embodiments, the modulated MEGI functional connectivity comprises decreased MEGI functional connectivity in the frontal cortex of the frontal lobe region of the brain. In some embodiments, the method further comprises treating the individual with tinnitus by delivering electrical, acoustic, and/or magnetic stimulation to the individual. In some embodiments, the method further comprises treating the individual with tinnitus by delivering electrical, acoustic, and/or magnetic signals to the individual. In some embodiments, the stimulation is synchronized stimulation. In some embodiments, the stimulation is pulsatile stimulation. In some embodiments, the at least one region of the brain is at least two regions of the brain. In some embodiments, the method further comprises recording auditory-evoked field (AEF) peak latency in the subject in response to a pure-tone stimulus, wherein the AEF peaks are recorded using a MEGI imaging (MEGI) device. In some embodiments, the determining further comprises determining if the AEF peak latency in the subject is above, below, or at a second reference level associated a second pathology profile of Tinnitus, wherein the second pathology profile comprises delayed latency of the AEF peaks in response to the pure-tone stimulus as compared to the second reference level. In some embodiments, the fMRI functional connectivity data comprises oscillating neural signals between the auditory cortex and the rest of the brain. In some embodiments, assessing the MEGI functional connectivity comprises assessing the hyposynchrony in the frontal cortex of the brain. In some embodiments, assessing the hyposynchrony in the frontal cortex of the brain comprises assessing the global connectivity of the frontal cortex of the brain with the rest of the brain. In some embodiments, the frontal cortex hyposynchrony magnitude is correlated with Tinnitus severity level. In some embodiments, assessing the MEGI functional connectivity comprises assessing shifts in MEGI bandwidth frequencies in the frontal cortex as associated with the one or more pathology profiles of Tinnitus. In some embodiments, decreased MEGI functional connectivity comprises decreased MEGI alpha-band activity ranging from 8-12 Hz. In some embodiments, assessing the fMRI functional connectivity comprises assessing coherence between: a) the caudate nucleus and the auditory cortex; b) the caudate nucleus and the frontal lobe; c) a combination thereof. In some embodiments, assessing the fMRI functional connectivity comprises assessing hypoconnectivity between the caudate nucleus and the frontal lobe. In some embodiments, assessing the fMRI functional connectivity comprises assessing hypoconnectivity between the caudate nucleus and the frontal lobe. In some embodiments, the one or more pathology profiles of Tinnitus is further associated with: a) modulated functional connectivity between the caudate nucleus and the cuneus region of the brain; b) modulated functional connectivity between the caudate nucleus and the superior lateral occipital cortex (sLOC); or c) modulated functional connectivity between the caudate nucleus and the anterior supramarginal gyrus (aSMG). In some embodiments, the modulated functional connectivity comprises increased functional connectivity between the caudate nucleus and the cuneus region of the brain. In some embodiments, modulated functional connectivity comprises increased functional connectivity between the caudate nucleus and the sLOC. In some embodiments, modulated functional connectivity comprises increased functional connectivity between the caudate nucleus body and the auditory cortex. In some embodiments, modulated functional connectivity comprises increased functional connectivity between the caudate nucleus head and the auditory cortex. In some embodiments, modulated functional connectivity comprises increased functional connectivity between the caudate nucleus and the aSMG. In some embodiments, AEFs are evoked by the pure-tone stimulus at 1 kHz. In some embodiments, the method further comprises acquiring a plurality of high-resolution MR images. In some embodiments, the plurality of high-resolution MR images is reconstructed into three-dimensional images. In some embodiments, the acquiring comprising acquiring the MEGI functional connectivity data with a resting-state MEGI imaging device (MEGI) with the subject's eyes closed. In some embodiments, the recording comprises collecting the AEF peaks with the MEGI device with the subject's eyes open. In some embodiments, the acquiring comprises acquiring the MEGI functional connectivity data with the subject's eyes closed.

In one aspect, the present disclosure relates to a method of analyzing images of the brain, the method comprising: providing a database, using logistic regression algorithms, that comprises one or more pathology profiles associated with Tinnitus with or without hearing impairment; receiving a plurality of functional magnetic resonance (fMR) images or functional magnetoencephalographic (MEG) images of at least one region of the brain; analyzing the plurality of fMRI or MEGI images to obtain fMRI and MEGI functional connectivity data; and comparing the fMRI or MEGI functional connectivity data from the fMRI or fMEGI images with the one or more pathology profiles. In some embodiments, the one or more pathology profiles is associated with acute or chronic tinnitus. In some embodiments, the one or more pathology profiles is associated with hearing impairment. In some embodiments, hearing impairment comprises: i) acute or chronic hearing loss; ii) symmetric or asymmetric hearing loss; or iii) a combination thereof. In some embodiments, the one or more pathology profiles is associated with Tinnitus with or without hearing impairment. In some embodiments, the one or more pathology profiles is derived from a plurality of fMRI or MEGI images of one or more subjects having the one or more pathology profiles. In some embodiments, the plurality of fMRI images are three dimensional images. In some embodiments, the plurality of MEGI images are three dimensional images. In some embodiments, the method further comprises receiving auditory-evoked field (AEF) data in response to a pure-tone stimulus. In some embodiments, the AEF data comprises AEF peaks corresponding to spatiotemporal auditory cortical activity. In some embodiments, the database further comprises AEF data associated with the one or more pathology profiles. In some embodiments, the method further comprises comparing latency of the AEF peaks in response to the pure-tone stimulus with the AEF data associated with the one or more pathology profiles.

One aspect of the present disclosure relates to a method of analyzing fMRI signals or MEGI signals of the brain. In some embodiments, the method comprises providing a database, using logistic regression algorithms, that comprises one or more pathology profiles associated with Tinnitus with or without hearing impairment; receiving functional fMRI signals or functional MEGI signals from at least one region of the brain; analyzing the plurality of fMRI or MEGI signals to obtain fMRI or MEGI functional connectivity data; and comparing the fMRI or MEGI functional connectivity data from the fMRI or MEGI signals with the one or more pathology profiles.

One aspect of the present disclosure relates to a multimodal automated system for determining the presence of Tinnitus in the subject, the system comprising: a functional magnetic resonance imaging (fMRI) device or a magnetoencephalographic imaging (MEGI) device; at least one memory storage medium configured to store functional connectivity data of the brain of the subject received from the fMRI or MEGI device; at least one processor operably coupled to the at least one memory storage medium, the at least one processor being configured to: i) process fMRI data or MEGI data recorded from at least one region of the brain in an individual, thereby generating fMRI functional connectivity data or MEGI functional connectivity data; ii) analyze fMRI or MEGI functional connectivity data, iii) prune, using logistic regression algorithms, the fMRI functional connectivity data or the MEGI functional connectivity data; iv) compare the fMRI or MEGI functional connectivity data obtained in step iii with functional connectivity data obtained from a database comprising one or more pathology profiles associated with Tinnitus; and v) determine if the individual has Tinnitus based on the data obtained in step iv. In some embodiments, the one or more pathology profiles is further associated with hearing impairment. In some embodiments, the processor is further configured to identify latencies of the auditory-evoked field (AEF) peaks recorded from the auditory cortex of the individual in response to a pure-tone stimulus. In some embodiments, the at least one region of the brain comprises the a) caudate nucleus region of the brain; b) caudate head region of the brain; c) caudate body region of the brain; d) auditory cortex region of the brain; e) frontal lobe region of the brain; f) superior occipital cortex region of the brain; g) cuneus region of the brain; or h) a combination thereof. In some embodiments, the MEGI functional connectivity data is recorded in the frontal cortex of the frontal lobe region of the brain. In some embodiments, the MEGI functional connectivity data is recorded in the left and right superior frontal gyrus region of the frontal lobe. In some embodiments, the processing fMRI data comprises linearly detrending and bandpass filtering the fMRI data or MEGI data. In some embodiments, the fMRI functional connectivity data comprises a plurality of images. In some embodiments, the MEGI functional connectivity data comprises a plurality of images. In some embodiments, the processor is further configured to define seed regions within the plurality of images: i) anatomically based on subdivisions of the caudate nucleus of the rest of the brain; and ii) functionally using localizers for the auditory cortex auditory-evoked field (AEF) data recorded from the auditory cortex of the individual in response to a pure-tone stimulus. In some embodiments, the processor is further configured to define seed regions using a statistical map and stereotactic coordinates of the at least one region of the brain. In some embodiments, the comparing further comprises comparing the AEF latency peaks from the individual with one or more latency peaks AEF latency peaks obtained from the database. In some embodiments, the logistic regression algorithm is a linear least squares regression, robust linear regression, support vector machine, k-means clustering, or ridge regression. In some embodiments, the logistic regression algorithm comprises a plurality of logistic regression models. In some embodiments, the logistic regression algorithm is a relevance vector machine that executes automatic feature pruning. In some embodiments, the logistic regression algorithm deploys variants of relevance vector machines to perform pruning. In some embodiments, the at least one or the plurality of logistic regression models comprises predictor variables of functional connectivity data. In some embodiments, the functional connectivity at each oscillatory frequency is quantified by averaging an imaginary component of coherence across a plurality of seeds.

One aspect of the present disclosure comprises a multimodal neuroimaging system. In some embodiments, the system comprises: a functional magnetic resonance imaging (fMRI) device or a magnetoencephalographic imaging (MEGI) device; at least one memory storage medium configured to store functional connectivity data of the brain of the subject received from the fMRI or MEGI device; and at least one processor operably coupled to the at least one memory storage medium, the at least one processor being configured to: i) process fMRI data or MEGI data recorded from at least one region of the brain in an individual, thereby generating fMRI functional connectivity data or MEGI functional connectivity data; ii) analyze fMRI or MEGI functional connectivity data, iii) prune, using logistic regression algorithms, the fMRI functional connectivity data or the MEGI functional connectivity data; iv) compare the fMRI or MEGI functional connectivity data obtained in step iii with functional connectivity data obtained from a database comprising one or more pathology profiles associated with Tinnitus; and v) determine if the individual has Tinnitus based on the data obtained in step iv.

One aspect of the present disclosure relates to a neuroimaging system. In some embodiments, the system comprises: a functional magnetic resonance imaging (fMRI) device; a processor; and a non-transient computer-readable medium comprising instructions that, when executed by the processor, cause the processor to: i) process fMRI functional connectivity data of a brain of an individual, thereby generating fMRI functional connectivity data for at least one region of the brain ii) analyze the fMRI functional connectivity data; and iii) determine if the individual has Tinnitus based on a binomial logistic regression model of functional connectivity between the caudate and auditory cortex region of the brain, wherein the binomial logistic regression model comprises functional connectivity values from bihemispheric caudate connectivity maps extracted from the ipsilateral posterior middle temporal gyrus of the brain.

One aspect of the present disclosure relates to a non-transitory computer-readable memory medium comprising instructions that when executed cause a processor to: i) process fMRI data or MEGI data recorded from at least one region of the brain in an individual, thereby generating fMRI functional connectivity data or MEGI functional connectivity data; ii) analyze fMRI or MEGI functional connectivity data, iii) prune, using logistic regression algorithms, the FMRI functional connectivity data or the MEGI functional connectivity data; iv) compare the fMRI or MEGI functional connectivity data obtained in step iii with functional connectivity data obtained from a database comprising one or more pathology profiles associated with Tinnitus; and v) determine if the individual has Tinnitus based on the data obtained in step iv.

One aspect of the present disclosure relates to a non-transitory computer-readable memory medium comprising instructions that when executed cause a processor to: i) process fMRI data recorded from at least one region of the brain in an individual, thereby generating fMRI functional connectivity data; ii) analyze fMRI functional connectivity data, iii) prune, using logistic regression algorithms, the fMRI functional connectivity data; iv) compare the fMRI functional connectivity data obtained in step iii with functional connectivity data obtained from a database comprising one or more pathology profiles associated with Tinnitus; and v) determine if the individual has Tinnitus based on the data obtained in step iv.

One aspect of the present disclosure relates to a non-transitory computer-readable memory medium comprising instructions that when executed cause a processor to: i) process MEGI data recorded from at least one region of the brain in an individual, thereby generating MEGI functional connectivity data; ii) analyze MEGI functional connectivity data, iii) prune, using logistic regression algorithms, the MEGI functional connectivity data; iv) compare the MEGI functional connectivity data obtained in step iii with functional connectivity data obtained from a database comprising one or more pathology profiles associated with Tinnitus; and v) determine if the individual has Tinnitus based on the data obtained in iv.

One aspect of the present disclosure relates to a method of treating Tinnitus in a subject, the method comprising: a) acquiring functional magnetic resonance imaging (fMRI) functional connectivity data or magnetoencephalographic imaging (MEGI) functional connectivity data of at least one of the caudate nucleus, the caudate head, the caudate body, the frontal lobe, and the auditory cortex regions of the brain of the subject; b) assessing the fMRI functional connectivity data or the MEGI functional connectivity data in at the at least one region of the brain; c) determining if the fMRI functional connectivity data or the MEGI functional connectivity data are above, below, or at a reference level associated with at least one or more pathology profiles of Tinnitus, wherein at least one pathology profile of Tinnitus comprises: i) modulated fMRI functional connectivity between the caudate nucleus and the rest of the brain as compared to the reference level; ii) modulated MEGI functional connectivity in the frontal lobe as compared to the reference level; or iii) modulated MEGI functional connectivity in the auditory cortex regions as compared to the reference level; and d) delivering electrical, acoustic, or magnetic stimulation in one or more of the caudate nucleus, the caudate head, the caudate body, the frontal lobe, and the auditory cortex regions of the brain to reduce tinnitus loudness in the individual. In some aspects, the electrical stimulation is deep brain stimulation (DBS). In some aspects, the electrical stimulation is macrostimulation In some aspects, magnetic stimulation is generated by at least one of a Low Field Magnetic Stimulator (LFMS), a Magnetic Resonance Imager (MRI), a Transcranial Magnetic Stimulator (TMS), a Neuro-EEG synchronization Therapy device, or a combination thereof. In some aspects, delivering stimulation comprises delivering one or more synchronized stimulations to the at least one or more of the caudate nucleus, the caudate head, the caudate body, the frontal lobe, and the auditory cortex regions of the brain. In some aspects, at least one synchronized stimulation comprises stimulation of multiple non-auditory pathways of 10 or more, 20 or more, or 30 or more locations across the caudate nucleus, the caudate head, the caudate body, the frontal lobe, and the auditory cortex regions of the brain. In some aspects, electrical stimulation is performed in one or more locations in the caudate body region of the brain. In some aspects, the electrical stimulation was performed in one or more locations in the caudate head or the brain.

The following examples are offered by way of illustration and not by way of limitation.

### BRIEF DESCRIPTION OF THE FIGURES

FIG. 1 depicts a Striatal Gate Model of Tinnitus (Larson and Cheung et al. 2012). Conscious awareness of auditory phantoms is contingent on associated corticostriatal signals passing through the dorsal striatum or caudate nucleus. Strength of phantom percept neural representations, external modulators, and mood-related circuits of the ventral striatum determine tinnitus severity.

FIG. 2 shows RS-fMRI images, collected at 7 Tesla, of each caudate nucleus with reciprocal patterns of functional connectivity and increased functional connectivity with auditory cortex in subjects with Tinnitus. Top row: Within-group averages for the left and right caudate seeds used to examine resting-state functional connectivity using RS-fMRI. Bottom row: Group comparison between subjects with Tinnitus and moderate hearing loss (TIN+HL) and subjects with moderate hearing loss alone (HL), without Tinnitus. The Tinnitus subjects show significant increases in resting-state connectivity between the caudate nucleus and primary auditory cortex (A1) for both left and right caudate seeds.

FIG. 3 shows RS-fMRI images, collected at 3 Tesla, of each primary auditory cortex (A1) with reciprocal patterns of functional connectivity and increased functional connectivity with the caudate striatum in subjects with Tinnitus. Top row: Resting-state network (RSN) of right and left primary auditory cortices (A1) show functional connectivity with each other in subjects with profound unilateral hearing loss or single-sided deafness (SSD). Bottom row: Group comparison between subjects with Tinnitus and SSD (TIN+SSD) and subjects with SSD alone, without Tinnitus. The Tinnitus subjects show significant increases in resting-state connectivity between the primary auditory cortex (A1) and the caudate nucleus for both left and right A1 seeds.

FIG. 4 shows RS-fMRI segmented images of the caudate nucleus, revealing distinct patterns of functional connectivity of each caudate segment. Upper row: 9 subdivisions defined by fMRI functional connectivity. Middle and lower panels: grand mean functional connectivity maps across all subjects with tinnitus and moderate hearing loss (TIN+HL) and subjects with moderate hearing loss alone (HL) for the 9 separate caudate subdivisions (Seed 1-9). Seed (5 mm radius sphere) locations are derived from centroid coordinates. Patterns of activation are bilateral and symmetric. Renderings are shown only for the left lateral surface (middle row) and left medial surface (lower row). Distinct networks are identifiable for each separate seed, confirming caudate segmentation into 9 separate subdivisions remains valid in chronic Tinnitus. All images are statistically thresholded (p<0.05) and superimposed using the CONN toolbox.

FIG. 5 shows RS-fMRI images comparing subjects with Tinnitus and moderate hearing loss (TIN+HL) to subjects with hearing loss alone (HL), where increased corticostriatal connectivity in chronic Tinnitus is specific to particular caudate subdivisions. Group comparison between subjects with TIN+HL and subjects with HL for the 9 separate subdivisions of the caudate nuclease (Seed 1-9). Top row: seed locations for each functional subdivision in the left (yellow) and right (pink) hemisphere. Middle row: comparison between the two cohorts (TIN+HL>HL) for seeds placed in the left hemisphere. Increased connectivity between the caudate and ipsilateral posterior middle temporal gyrus of auditory cortex is specific to seed location 7 (p<0.005) for the TIN+HL cohort. Bottom row: comparison between the two cohorts (TIN+HL>HL) for seeds placed in the right hemisphere. Increased connectivity between the caudate and ipsilateral posterior middle temporal gyrus of the auditory cortex is specific to seed location 6 (p<0.005) for the TIN+HL cohort. All images are statistically thresholded and superimposed using the CONN toolbox.

FIG. 6 shows images of 20 caudate nucleus locations that were systemically interrogated by positioning a deep brain stimulation (DBS) lead at the desired locale and delivering broad stimulation under different frequency and intensity parameters. Intraoperative direct electrical stimulation of the caudate body, positioned posterior to the caudate head, is more likely to modulate tinnitus loudness acutely in subjects. Left: left hemisphere sagittal image shows the single responder (green) positioned at the caudate head. Middle: axial image shows the spatial distribution of responders (green) and non-responders (red) in the 2 hemispheres. Right: right hemisphere sagittal image shows all non-responders (red) positioned at the caudate head. All images are in Montreal Neurological Institute (MNI) coordinates.

FIG. 7 shows RS-fMRI images comparing functional connectivity profiles of responders versus non-responders by seeding the centroids of respective clusters in 20 chronic Tinnitus subjects with Tinnitus Functional Index scores greater than 50, indicative at moderate disease severity or worse. Group comparisons between resting-state networks collected at 3 Tesla of acute tinnitus modulation by DBS responders at centroid local (caudate body) and non-responders at a separate centroid locale (caudate head) show that auditory cortex (left: left hemisphere; right: right hemisphere) has increased connectivity with the more posteriorly positioned caudate body subdivision (p<0.05).

FIG. 8 shows RS-MEGI of alpha-band (8-12 Hz) functional connectivity of frontal cortex predicts Tinnitus severity. Whole-brain analysis of chronic Tinnitus subjects shows that functional connectivity strength of the left superior frontal gyrus is correlated with tinnitus severity, as measured by the Tinnitus Functional Index (TFI) score (p<0.05, corrected for multiple comparisons). Scatter plot of functional connectivity strength of the left superior frontal gyrus and TFI scores shows significant negative correlation (r−0.744, p<0.05).

FIG. 9 shows RS-MEGI of alpha-band (8-12 Hz) functional connectivity predicts cognitive performance on the Montreal Cognitive Assessment (MoCA). Statistically significant maps of correlations (p<0.05 corrected for five voxel clusters minimum) between MoCA and alpha-band functional connectivity reveal increased functional connectivity in left A) middle temporal gyrus and B) occipital lobe is correlated with reduced cognitive performance. Scatterplots of the negative correlations in these two regions are also shown.

FIG. 10 shows RS-MEGI of alpha-band (8-12 Hz) functional connectivity predicts cognitive performance on the Montreal Cognitive Assessment (MoCA). Statistically significant map of correlations (p<0.05 corrected for five voxel clusters minimum) between MoCA and alpha-band functional connectivity reveal increased functional connectivity in the right A) middle temporal gyrus and B) mesial anterior cingulate cortex is correlated with reduced cognitive performance. Scatterplots of the negative correlations in these two regions are also shown.

FIG. 11 shows task-based MEGI delayed latency of auditory evoked field (AEF) peaks in response to 1 kHz tones in Tinnitus in a group comparison between subjects with Tinnitus and moderate hearing loss (TIN+HL) and subjects with moderate hearing loss alone (HL). AEF latencies were averaged across the left and right ears for TIN+HL (red) and HL (blue). TIN+HL show longer AEF latencies when compared to HL alone (p<0.05), indicating chronic tinnitus is associated with delayed sound processing in auditory cortex.

FIG. 12 depicts a plot illustrating an algorithm deployment in a patient dataset. Bayesian machine learning enabled MEGI diagnostic tool classifies dementia variants for primary progressive aphasia (PPA). Receiver operating characteristic (ROC) curves for pairwise comparisons of all three variants (IvPPA, svPPA, and nfvPPA) are displayed. Pairwise discriminations of dementia variants based on the resting-state functional connectivity are shown: (A) 1vPPA vs nfvPPA; (B) nfvPPA vs. svPPA; (C) svPPA vs. 1vPPA. Each subplot displays three ROC curves: delta-theta (2-8 Hz; yellow line); alpha (2-8 Hz; blue line); beta (12-30 Hz; red line) oscillations. Each logistic regression model includes predictor variables of functional connectivity imaging data from the pair of PPA variants. Functional connectivity at each oscillatory frequency is quantified by taking the average of the imaginary component of the coherence (represented in the complex plane) across all voxels. The imaginary component of coherence is invariant to spurious instantaneous coupling due to volume conduction effects. AUC=Area Under the Curve (confidence interval within parentheses); PPA=primary progressive aphasia; 1vPPA=logopenic variant; nfvPPA=non-fluent variant; svPPA=semantic variant. These results demonstrate that RS-MEGI functional connectivity can be potentially used as a diagnostic biomarker.

FIG.13 depicts a plot for an exemplary neuroimaging-based Tinnitus diagnostic tool. A logistic regression model predicts Tinnitus accurately based on functional connectivity of bihemispheric caudate with auditory cortices in a cohort of subjects with moderate hearing loss, some with Tinnitus and some without Tinnitus. The area under the ROC curve=0.836.

FIG. 14 shows metabolite ratios (GABA/NAA+NA) collected using 7T MR spectroscopy for seeds placed in the left and right basal ganglia for subjects with Tinnitus and hearing loss (TIN+HL, COHORT 1 in red) and hearing loss only (HL, COHORT 2 in blue). GABA/NAA+NA ratio is reduced in the TIN+HL (COHORT 1). GABA concentration alteration may be a neurochemical marker of a dysfunctionally permissive dorsal striatal gate in chronic tinnitus.

FIG. 15 shows RS-fMRI images of hypoconnectivity between the caudate nucleus and frontal lobe distinguishes subjects with Tinnitus with hearing loss from those with hearing loss alone. Group comparison of RS-fMRI functional connectivity of the nine subdivisions (Seed 1-9) of the left caudate (Top Row) and right caudate (Bottom Row) is made using 3 Tesla fMRI. Significant (p<0.001) decreases in functional connectivity are observed in the Tinnitus with hearing loss group: 1) Seed 4 of the left caudate and the paracingulate gyrus (ParCing) of the frontal lobe, and 2) Seeds 4 and 6 of the right caudate and ParCing (in blue). Statistical maps are thresholded and generated using the CONN toolbox.

FIG. 16 shows RS-fMRI images of strength of connectivity between caudate nucleus and nonauditory structures is corrected with tinnitus severity domains. Top: connectivity strength between caudate nucleus and cuneus is correlated with relaxation difficulty attributed to tinnitus. Middle: connectivity strength between caudate nucleus and superior lateral occipital cortex (sLOC) is correlated with control difficulty attributed to tinnitus. Bottom: connectivity strength between caudate nucleus and anterior supramarginal gyrus (aSMG) is correlated with control difficulty attributed to tinnitus.

FIG. 17 shows 20 locations of deep brain stimulation (DBS) electrode placement with macrostimulation displayed in MNI space. Caudate nucleus locations with (green) and without (red) tinnitus loudness reduction are displayed. Within the caudate head, there is 1 location with tinnitus loudness reduction and 15 locations without.

FIG. 18 shows an anteroposterior map of the caudate nucleus for tinnitus modulation. The caudate head is anterior (positive, left) and the body is posterior (negative, right). Data are aggregated from both hemispheres. The outcome of tinnitus loudness interrogation at each anteroposterior coordinate is coded by a box. Increase and decrease in tinnitus loudness modulation is more strongly expressed for MNI coordinates between −8 and −15 (caudate body).

FIG. 19 shows a heat map display of functional connectivity of the left posterior caudate body seed compared to the left anterior caudate head seed. The left caudate body demonstrates increased auditory corticostriatal functional connectivity with both superior temporal gyri. Yellow indicates relatively higher connectivity compared to that indicted by orange. Positive contrast was performed using second-level analysis in the CONN toolbox, with a height threshold of p<0.05 and cluster correction threshold of p<0.05, using a false discovery rate correction.

FIG. 20 shows Table 1 with a summary of baseline characteristics in 20 study participants.

FIG. 21 shows Table 2 with intraoperative caudate nucleus stimulation parameters.

FIG. 22 shows Table 3 with acute tinnitus loudness modulation by caudate nucleus stimulation.

### DEFINITIONS

The term “assessing” includes any form of measurement and includes determining if an element is present or not. The terms “determining”, “measuring”, “evaluating”, “assessing” and “assaying” are used interchangeably and include quantitative and qualitative determinations. Assessing may be relative or absolute.

A “plurality” contains at least 2 members. In certain cases, a plurality may have at least 10, at least 100, at least 1000, at least 10,000, at least 100,000, at least 106, at least 107, at least 108 or at least 109 or more members.

An “individual” or “subject” as used herein, may be any suitable animal amenable to the methods and techniques described herein, where in some cases, the individual may be a vertebrate animal, including a mammal, bird, reptile, amphibian, etc. The individual may be any suitable mammal, e.g., human, mouse, rat, cat, dog, pig, horse, cow, monkey, non-human primate, etc. In some cases, the subject is a human.

“Functional connectivity”, as used herein, may refer to the magnitude of correlation or to the strength of synchrony between a seed and target brain region, or to the average synchrony between a particular brain region and the rest of the brain.

“Seed” as used herein, may refer to an anatomical or functional region of interest (ROI), coordinates, or location of brain activity. A seed may be used interchangeably with signals from a voxel, or cluster of voxels used to calculate correlations with other voxels, or seeds, of the brain.

The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, whole blood, blood fractions such as plasma and serum, and the like.

“Resting” or “resting-state”, as used herein, may refer to an individual who is not performing an explicit, or an externally prompted task. Resting-state functional activity data, such as resting-state fMRI data, may refer to functional activity data collected from an individual who has not been instructed to perform an explicit task requiring active engagement during data acquisition.

“Task-based MEGI” or “Task-based functional connectivity”, as used herein, may refer to the activity of regions of the brain during execution of tasks, or in response to stimuli, such as pure tones.

“Auditory Evoked Field (AEF)”, as used herein, may refer to a form of neural activity that is induced by an auditory stimulus and recorded using a MEGI device or MRI device.

“Hyposynchrony”, as used herein, may refer to decreased synchronization of neuronal activity between a specific brain region and the rest of the brain, separate brain regions or subdivisions of a particular brain region.

“Hypersynchrony”, as used herein, may refer to increased synchronization of neuronal activity between a specific brain region and the rest of the brain, separate brain regions or subdivisions of a particular brain region.

“Hypoconnectivity”, as used herein, may refer to decreased functional connectivity between a specific brain region and the rest of the brain, separate brain regions or subdivisions of a particular brain region.

“Hyperconnectivity”, as used herein, may refer to increased functional connectivity between a specific brain region and the rest of the brain, separate brain regions or subdivisions of a particular brain region.

### DETAILED DESCRIPTION

The present disclosure relates to multimodal neuroimaging-based systems, devices, and methods for analyzing brain function connectivity, synchrony, and spatiotemporal activity using functional magnetic resonance imaging (fMRI) and magnetoencephalographic imaging (MEGI). More specifically, present disclosure relates to detecting and/or monitoring Tinnitus in a subject. Also provided are systems configured for performing the disclosed methods and computer readable medium storing instructions for performing steps of the disclosed methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

The present disclosure provides a method of detecting, monitoring, and/or diagnosing Tinnitus in a subject using a multimodal imaging approach. The present methods provide biomarkers to measure and/or monitor Tinnitus severity objectively. The present methods are also useful in detecting and/or diagnosing hearing impairment or primary progressive aphasia.

fMRI

Aspects of the present methods include performing fMRI of at least one region of the brain of a subject. In some cases, performing fMRI of at least one region of the brain comprises collecting fMRI functional activity data. In some cases, fMRI functional connectivity data is acquired using an fMRI device. In some cases, the fMRI device is a resting-state fMRI device. In some cases, fMRI functional connectivity data is resting-state fMRI functional connectivity data.

Aspects of the present methods include acquiring fMRI functional connectivity data in at least one region of the brain. In some cases, the fMRI functional connectivity data is acquired in at least two regions of the brain. In some cases, the fMRI functional connectivity data is acquired in at least three, at least four, at least five, at least six, at least seven, at least eight, at least nine, or at least ten regions of the brain. In some cases, the functional connectivity data is acquired in the entire brain.

Functional connectivity data may be acquired from any suitable brain region. Suitable brain regions include, without limitation, caudate dorsal striatum, caudate head, nucleus accumbens, caudate body, auditory cortex, frontal lobe, thalamus, non-auditory cortex, superior occipital lobe, ventral tegmental area (VTA), prefrontal cortex (PFC), amygdala, substantia nigra, ventral pallidum, globus pallidus, ventral striatum, subthalamic nucleus, anterior caudate putamen, globus pallidus external, anterior supramarginal gyrus, globus pallidus internal, hippocampus, dentate gyrus, cingulate gyrus, entorhinal cortex, olfactory cortex, motor cortex, cerebellum, lateral occipital cortex, cuneus, or a combination thereof.

In some cases, acquiring fMRI functional connectivity data comprises acquiring fMRI data from the subject's brain. In some cases, the imaging data is reconstructed into three-dimensional (3D) images. Non-limiting programs that may be used to reconstruct 3D images include Matlab®, Voloom (microDimensions, Munich, Germany), Imaris, Image-Pro Premier 3D (Media Cybernetics, Rockville, Md., USA), or any available 3D imaging reconstruction software. In some cases, acquiring fMRI functional connectivity data comprises reconstructing, from a plurality of acquired MR image, 3D MR images of the subject's brain by starting from a seed location within the brain and building the model outward to the surface of the brain. In some cases, the seed is placed in the right and left primary auditory cortices. In some cases, the seed is placed in the center of the caudate nucleus. In some cases, the seed is placed at 9 subdivisions of the caudate nucleus. In some cases, a 1 mm radius sphere seed, a 2 mm radius sphere seed, a 3 mm radius sphere seed, a 4 mm radius sphere seed, a 5 mm radius sphere seed, a 6 mm radius sphere seed, a 7 mm radius sphere seed, an 9 mm radius sphere seed, and/or a 10 mm radius sphere seed is positioned at the centroid coordinate for each subdivision. In some cases, the subdivisions of the caudate nucleus exhibit distinct functional connectivity patterns between subjects with Tinnitus and subjects without Tinnitus.

In some cases, fMRI functional connectivity data of the present methods comprises a plurality of images. In some cases, a high-resolution anatomical MRI is acquired. In some cases, functional connectivity data includes the corticostriatal connectivity data between the auditory cortex and the dorsal striatal region of the brain. In some cases, the fMRI functional connectivity data includes oscillating neural signals between the auditory cortex and the rest of the brain in the subject. In some cases, the fMRI functional connectivity data includes oscillating neural signals between the caudate nucleus and the rest of the brain in the subject.

In some cases, seed regions are defined both anatomically and functionally using localizers for auditory cortex obtained from task-based MEGI. In some cases, a processor is configured to define the seed regions within the plurality of images anatomically and functionally using localizers for auditory cortex recorded from AEF peak signals from a MEGI device.

In some cases, functional connectivity data is fMRI data collected from the fMRI device. In some cases, functional connectivity data is fMRI data. In some cases, performing fMRI of the brain comprises collecting repetitions of spontaneous 1 Tesla or more, 2 Tesla or more, 3 Tesla or more, 4 Tesla or more, 5 Tesla or more, 6 Tesla or more, 7 Tesla or more, 8 Tesla or more, 9 Tesla or more, or 10 Tesla or more fMRI data for a period of time. In some cases, collecting comprises collecting repetitions of spontaneous 3 Tesla fMRI data. In some cases, collecting comprises collecting repetitions of spontaneous 7 Tesla fMRI data.

In some cases, performing fMRI of the brain comprises collecting repetitions of spontaneous 1 Tesla or more, 2 Tesla or more, 3 Tesla or more, 4 Tesla or more, 5 Tesla or more, 6 Tesla or more, 7 Tesla or more, 8 Tesla or more, 9 Tesla or more, or 10 Tesla or more fMRI data for a period of time. In some cases, the repetitions range from 1-100 repetitions, 100-200 repetitions, 200-300 repetitions, 300-400 repetitions, 400-500 repetitions, 500-600 repetitions, 600-700 repetitions, 700-800 repetitions, 800-900 repetitions, or 900-1000 repetitions. In some cases, the repetitions range from 200-210 repetitions, 210-220 repetitions, 220-230 repetitions, 230-240 repetitions, 240-250 repetitions, 250-260 repetitions, 270-280 repetitions, 280-290 repetitions, 290-300 repetitions, 300-310 repetitions, 310-320 repetitions, 320-330 repetitions, 330-340 repetitions, or 340-350 repetitions. In some cases, the repetitions include at least 240 repetitions. In some cases, the repetitions include at least 245 repetitions. In some cases, the repetitions include 250 repetitions. In some cases, the repetitions include at least 255 repetitions. In some cases, the repetitions include at least 260 repetitions. In some cases, the repetitions include 265 repetitions. In some cases, the repetitions include at least 270 repetitions. In some cases, the repetitions include at least 275 repetitions. In some cases, the repetitions include at least 280 repetitions. In some cases, the repetitions include at least 285 repetitions. In some cases, the repetitions include at least 290 repetitions. In some cases, the repetitions include at least 295 repetitions. In some cases, the repetitions include at least 300 repetitions. In some cases, the repetition time (TR) is 50 ms, 100 ms, 150 ms, 200 ms, 250 ms, 300 ms, 350 ms, 400 ms, 450 ms, or 500 ms. In some cases, the TR ranges from 0-50 ms, 50-100 ms, 100-150 ms, 150-200 ms, 200-250 ms, 250-300 ms, 300-350 ms, 350-400 ms, 400-450 ms, or 450-500 ms.

In some cases, the period of time for collecting repetitions of spontaneous Tesla fMRI data include 1 minute, 2 minutes, 3 minutes, 4 minutes, 5 minutes, 6 minutes, 7 minutes, 8 minutes, 9 minutes, or 10 minutes. In some cases, the period of time for collecting repetitions of spontaneous Tesla fMRI data include 1-5 minutes, 5-10 minutes, 10-15 minutes, 15-20 minutes, 20-25 minutes, 25-30 minutes, 30-35 minutes, 35-40 minutes, 40-45 minutes, 45-50 minutes, 50-55 minutes, or 55-60 minutes.

In some cases, performing fMRI of the brain comprises collecting spontaneous fMRI data for a period of time with a gradient EPI sequence (2.0 mm×2.0 mm×2.0 mm slides, TR=2000 ms, TE=28 ms). In some cases, collecting spontaneous fMRI data with a gradient readout echo sequence (GRE), and a standard two-dimensional fast spin T2 weight sequence. In some cases, the present method further comprises acquiring a plurality of high-resolution MR images, a gradient readout echo sequence, and a standard two-dimensional fast spin T2 weight sequence. In some cases, collecting or recording of fMRI functional connectivity data comprises collecting fMRI functional connectivity data, wherein data is linearly detrended and bandpass filtered (2nd-order Butterworth; 0/1=0.008 Hz) prior to functional connectivity analysis.

In some cases, performing fMRI of the brain is performed with the subject's eyes closed. In some cases, performing fMRI of the brain is performed with the subject's eyes open. In some cases, performing fMRI of the brain is performed with the subject's in a supine position. In some cases, performing fMRI of the brain is performed with the subject's eyes open without any instruction to perform an explicit task requiring active engagement during data acquisition.

In some cases, assessing the fMRI functional connectivity data comprises evaluating the spatial extent and amplitude of fMRI connectivity networks. In some cases, assessing the fMRI functional connectivity data comprises assessing functional connectivity data seeded from the basal ganglia and auditory cortex of the brain. In some cases, the fMRI functional connectivity data is assessed using standard bivariate metrics. In some cases, the standard bivariate metrics comprise correlation and coherence. In some cases, assessing the fMRI functional connectivity data comprises evaluating the spatial extent and amplitude of fMRI connectivity networks, seeded from the basal ganglia and auditory cortex of the brain using standard multivariate metrics, such as, but not limited to independent components analysis. In some cases, assessing the fMRI functional connectivity data comprises assessing correlations in coherence between the caudate nucleus and the auditory cortex; the caudate nucleus and the frontal lobe; and/or a combination thereof. In some cases, assessing the fMRI functional connectivity data comprises assessing coherence between: the caudate nucleus and the auditory cortex; the caudate nucleus and the frontal lobe; or a combination thereof.

In some cases, acquiring fMRI functional connectivity data comprises acquiring fMRI signals. In some cases, fMRI functional connectivity of the resting brain is represented in fMRI by synchronously fluctuating, low frequency (<0.1 Hz) blood oxygenation level dependent (BOLD) signals. Emerging from these intrinsic signals are consistent, spatially distinct neural systems that mirror spatial representations found in task-based studies. In some cases, fMRI detects interregional temporal correlations of BOLD signal fluctuations. In some cases, regions whose BOLD signal fluctuations show a high degree of temporal correlation may constitute a tightly coupled neural network. In some cases, consistent, spatially distinct neural systems that mirror spatial representations in task-based studies can be analyzed from BOLD signals. BOLD signals operate on a time scale of several seconds. In some cases, fMRI data of the present methods comprises synchronously fluctuating, low frequency blood oxygenation dependent BOLD signals. BOLD-MR is an imaging tool that is sensitive to specific relaxation rates which are influenced by deoxyhemoglobin. BOLD-MRI contrast is derived from the inherent paramagnetic contrast of deoxyhemoglobin using T2* weighted images (Howe et al., 2001; Turner, 1997). In some cases, a gradient readout sequence (GRE) and a standard 3D T2* weighted sequence will be acquired in the subject at 0.352×0.352 mm voxel size and 512×512 matrix over an 18 cm field-of-view (FOV). In some cases, the matrix is over a 2 mm FOV, a 4 mm FOV, a 6 mm FOV, an 8 mm FOV, a 10 mm FOV, a 12 mm FOV, a 14 mm FOV, a 16 mm FOV, an 18 mm FOV, a 20 mm FOV, a 22 mm FOV, or a 24 mm FOV. The MR signal of blood is modulated by the ratio of oxyhemoglobin and deoxyhemoglobin, where changes in blood oxygen levels are observed as signal changes from the baseline. In the BOLD method the fact that oxyhemoglobin and deoxyhemoglobin are magnetically different is exploited. Oxyhemoglobin is diamagnetic whereas deoxyhemoglobin is paramagnetic. As deoxyhemoglobin is paramagnetic, it alters the T2* weighted magnetic resonance image signal. Thus, deoxyhemoglobin is sometimes referred to as an endogenous contrast enhancing agent, and serves as the source of the signal for fMRI. Imaging methods using BOLD signals of fMRI are described in U.S. Pat. Nos. 9,144,392 and 7,715,901, each of which are incorporated herein by reference.

In some cases, acquiring fMRI functional connectivity data comprises acquiring BOLD signals and fMRI images. In some cases, assessing fMRI functional connectivity data comprises assessing BOLD signals and fMRI images. In some cases, assessing fMRI functional connectivity data comprises assessing processed BOLD signals and reconstructed three-dimensional fMRI images. In some cases, assessing fMRI functional connectivity comprises assessing BOLD signals and reconstructed three-dimensional fMRI images using a three-dimensional tomographic map.

In some cases, fMRI functional connectivity data patterns are assessed and/or analyzed by defining seed regions using functional brain organization maps. In some cases, the seed regions are defined using stereotactic coordinates of a three-dimensional space in the brain. In some cases, the seed regions are defined using a three-dimensional statistical map.

In some cases, assessing and/or analyzing fMRI functional connectivity data comprises extracting connectivity values (i.e. correlation and/or coherence coefficients) from three-dimensional connectivity maps. Non-limiting examples of producing functional brain organization maps are described in U.S. Pat. No. 9,662,039, which is hereby incorporated by reference in its entirety.

In some cases, assessing fMRI functional connectivity data comprises analyzing the coordination and synchrony of the fMRI functional connectivity data between two brain regions.

In some cases, assessing fMRI functional connectivity data comprises assessing patterns of abnormal connectivity between the caudate nucleus and a separate region of the brain. In some cases, abnormal connectivity comprises increased fMRI functional connectivity between the caudate nucleus and the frontal lobe regions of the brain. In some cases, abnormal connectivity comprises decreased fMRI functional connectivity between the caudate nucleus and the frontal lobe regions of the brain. In some cases, abnormal connectivity comprises increased fMRI functional connectivity between the caudate nucleus and the auditory cortex regions of the brain. In some cases, abnormal connectivity comprises decreased fMRI functional connectivity between the caudate nucleus and the auditory cortex regions of the brain. In some cases, abnormal connectivity comprises increased functional connectivity between the caudate nucleus and the cuneus region of the brain. In some cases, abnormal connectivity comprises decreased functional connectivity between the caudate nucleus and the cuneus region of the brain. In some cases, abnormal connectivity comprises increased functional connectivity between the caudate nucleus and the superior lateral occipital cortex (sLOC). In some cases, abnormal connectivity comprises decreased functional connectivity between the caudate nucleus and the superior lateral occipital cortex (sLOC). In some cases, abnormal connectivity comprises increased functional connectivity between the caudate nucleus and the anterior supramarginal gyrus (aSMG). In some cases, abnormal connectivity comprises decreased functional connectivity between the caudate nucleus and the anterior supramarginal gyrus (aSMG).

In some cases, assessing fMRI functional connectivity data comprises assessing patterns of abnormal connectivity between the caudate nucleus and the auditory cortex of the brain in the subject. In some cases, assessing fMRI functional connectivity data comprises assessing patterns of abnormal corticostriatal connectivity between the caudate nucleus and a separate region of the brain.

In some cases, assessing fMRI functional connectivity data comprises assessing hypoconnectivity and/or hyperconnectivity between the caudate nucleus and the frontal lobe regions of the brain. In some cases, assessing fMRI functional connectivity comprises assessing functional connectivity strength between the caudate nucleus and a separate region of the brain. In some cases, assessing fMRI functional connectivity comprises assessing functional connectivity strength between the caudate nucleus and the rest of the brain. In some cases, assessing fMRI functional connectivity comprises assessing the magnitude of functional connectivity between the caudate nucleus and a separate region of the brain. In some cases, assessing fMRI functional connectivity comprises assessing the magnitude of functional connectivity between the caudate nucleus and the frontal lobe region of the brain. In some cases, assessing fMRI functional connectivity data comprises assessing the strength of connectivity between the caudate nucleus and non-auditory structures. In some cases, the strength of connectivity between the caudate nucleus and non-auditory structures is correlated with tinnitus severity domains. In some cases, an increase in functional connectivity between the caudate nucleus and a separate region of the brain (e.g. frontal lobe, cuneus, superior lateral occipital cortex, anterior supramarginal gyrus, auditory cortex) is correlated with an increase in Tinnitus Functional Index (TFI). In some cases, assessing the fMRI functional connectivity data comprises comparing the fMRI functional connectivity data with the TFI. A non-limiting example of the TFI can be found in Meikle et al., 2012 (Meikle, M B et al. 2012 Ear Hear. March-April; 33(2):153-76), which is hereby incorporated by reference in its entirety. In some cases, the fMRI functional connectivity data is correlated with a TFI to determine if the subject has Tinnitus. In some cases, the fMRI functional connectivity data is correlated with a TFI domain (e.g. difficulty with relaxation, sense of control, etc.) to determine the severity level of that particular domain in a subject with Tinnitus.

Aspects of the present methods include determining if the fMRI functional connectivity data is above, below, or at a reference level associated with at least one or more pathology profiles of Tinnitus. In some cases, at least one pathology profile of Tinnitus comprises modulated fMRI functional connectivity between the caudate nucleus and the rest of the brain as compared to the reference level. In some cases, at least one pathology profile of Tinnitus comprises modulated fMRI functional connectivity between the caudate nucleus and the rest of the brain; modulated fMRI functional connectivity between the caudate nucleus and the frontal lobe regions of the brain as compared to the reference level; modulated fMRI functional connectivity between the caudate nucleus and the auditory cortex regions of the brain as compared to the reference level; modulated MEGI functional connectivity in the frontal lobe as compared to the reference level; and/or modulated MEGI functional connectivity in the auditory cortex regions as compared to the reference level. In some cases, modulated fMRI functional connectivity comprises an increase in functional connectivity as compared to the reference level. In some cases, modulated fMRI functional connectivity comprises a decrease and/or reduction in functional connectivity as compared to the reference level. In some cases, modulated MEGI functional connectivity comprises an increase in functional connectivity as compared to the reference level. In some cases, modulated MEGI functional connectivity comprises a decrease and/or reduction in functional connectivity as compared to the reference level.

In some cases, the at least one pathology profile comprises at least two pathology profiles. In some cases, the at least one pathology profile comprises at least three pathology profiles. In some cases, the at least one pathology profile comprises at least two, at least three, at least four, at least five, at least six, at least seven, at least eight, at least nine, or at least ten pathology profiles.

In some cases, the reference level comprises one reference level. In some cases, the reference level comprises two reference levels. In some cases, the reference level comprises three reference levels. In some cases, the reference level comprises at least one, at least two, at least three, at least four, at least five, at least six, at least seven, at least eight, at least nine, or at least ten reference levels. In some cases, the reference level comprises a first, a second, a third, a fourth, a fifth, a sixth, a seventh, an eighth, a ninth, and/or a tenth reference level.

In some cases, at least one pathology profile of Tinnitus comprises modulated fMRI functional connectivity between the caudate nucleus and the frontal lobe regions of the brain as compared to a first reference level; modulated fMRI functional connectivity between the caudate nucleus and the auditory cortex regions of the brain as compared to a second reference level; and/or modulated MEGI functional connectivity in the frontal lobe as compared to a third reference level. In some cases, the first, the second, and the third reference level are the same. In some cases, the first, the second, and the third reference level are different.

In some cases, the strength and/or magnitude of functional connectivity between the caudate nucleus and non-auditory structures is correlated with tinnitus severity domains. In some cases, functional connectivity strength between the caudate nucleus and the cuneus is correlated with relaxation difficulty attributed to Tinnitus. In some cases, functional connectivity strength between the caudate nucleus and the superior lateral occipital cortex is correlated with control difficulty attributed to tinnitus. In some cases, the functional connectivity strength between the caudate nucleus and anterior supramarginal gyrus is correlated with control difficulty attributed to tinnitus.

In some cases, the one or more pathology profiles comprises modulated fMRI functional connectivity strength and/or magnitude between the caudate nucleus and non-auditory structures. In some cases, the one or more pathology profiles comprises an increase in functional connectivity strength between the caudate nucleus and a separate region of the brain (e.g. cuneus, superior lateral occipital cortex, anterior supramarginal gyrus). In some cases, the one or more pathology profiles comprises a decrease and/or reduction in functional connectivity strength between the caudate nucleus and a separate region of the brain (e.g. cuneus, superior lateral occipital cortex, anterior supramarginal gyrus).

In some cases, assessing and/or determining the fMRI functional connectivity comprises comparing patterns of functional connectivity in the brain of the subject with a database that includes one or more patterns of functional connectivity in the brain associated with subjects without Tinnitus, subjects with mild Tinnitus, subjects with moderate Tinnitus, subjects with severe Tinnitus, subjects with Tinnitus and symmetric and/or asymmetric hearing impairment, subjects with Tinnitus and hearing impairment, and/or a combination thereof. In some cases, hearing impairment comprises acute and/or chronic hearing loss; symmetric and/or asymmetric hearing loss; and/or a combination thereof. In some cases, Tinnitus can include acute or chronic tinnitus.

In some cases, assessing and/or determining the fMRI functional connectivity comprises providing a database that provides one or more pathology profiles associated with Tinnitus or hearing impairment. In some cases, the one or more pathology profiles comprises patterns of functional connectivity associated with subjects without Tinnitus and/or hearing loss, subjects with mild Tinnitus, subjects with moderate Tinnitus, subjects with severe Tinnitus, subjects with Tinnitus and symmetric and/or asymmetric hearing impairment, subjects with Tinnitus and hearing impairment, and/or a combination thereof. In some cases, hearing impairment comprises acute and/or chronic hearing loss; symmetric and/or asymmetric hearing loss; and/or a combination thereof. In some cases, Tinnitus can include acute or chronic tinnitus. In some cases, hearing impairment can include acute or chronic hearing loss, and/or symmetric or asymmetric hearing loss.

Aspects of the present methods include a method of detecting Tinnitus in a subject, the method comprising acquiring and assessing fMRI functional connectivity data to determine if the fMRI functional connectivity data is above, below, or at a reference level associated with one or more pathology profiles of Tinnitus. In some cases, the method further comprises acquiring and assessing MEGI functional connectivity data is above, below, or at a reference level associated with one or more pathology profiles of Tinnitus.

Aspects of the present methods include a method of treating or reducing Tinnitus in a subject. In some cases, the method includes a) acquiring functional magnetic resonance imaging (fMRI) functional connectivity data of at least one of the caudate nucleus, the caudate head, the caudate body, the frontal lobe, and the auditory cortex regions of the brain of the subject; b) assessing the fMRI functional connectivity data in at the at least one region of the brain; c) determining if the fMRI functional connectivity data are above, below, or at a reference level associated with at least one or more pathology profiles of Tinnitus, wherein at least one pathology profile of Tinnitus comprises: i) modulated fMRI functional connectivity between the caudate nucleus and the rest of the brain as compared to the reference level; and d) delivering electrical, acoustic, or magnetic stimulation in one or more of the caudate nucleus, the caudate head, the caudate body, the frontal lobe, and the auditory cortex regions of the brain to reduce tinnitus loudness in the individual. In some cases, the fMRI functional connectivity data is above a reference level associated with at least one or more pathology profiles of Tinnitus. In some cases, the fMRI functional connectivity data is below a reference level associated with at least one or more pathology profiles of Tinnitus. In some cases, the fMRI functional connectivity data is at a reference level associated with at least one or more pathology profiles of Tinnitus.

Aspects of the present methods include a method of treating or reducing tinnitus in an individual. In some cases, the method comprises delivering electrical, acoustic, and/or magnetic stimulation to the individual to treat or reduce tinnitus. In some embodiments, the method further comprises treating or reducing the individual with tinnitus by delivering electrical, acoustic, and/or magnetic signals to the individual. In some embodiments, the stimulation is synchronized stimulation. In some embodiments, the stimulation is pulsatile stimulation. In some cases, the treatment is acoustic stimulation. In some cases, the treatment is electrical stimulation. In some cases, the stimulation is macrostimulation. In some cases, the stimulation is magnetic stimulation. In some cases, the acoustic stimulation utilizes sound wave cancellation techniques. Non-limiting examples of electrical, acoustic, or magnetic stimulation treatments of Tinnitus can be found in U.S. Pat. Nos.: 6,210,321, 9,649,502, 10,265,527, 8,934,967, 6,610,019, and 9,242,067, which are hereby incorporated by reference in their entirety.

In some cases, performing stimulation comprises delivering one or more synchronized stimulations to the at least one or more of the caudate nucleus, the caudate head, the caudate body, the frontal lobe, and the auditory cortex regions of the brain. In some cases, at least one synchronized stimulation comprises stimulation of multiple non-auditory pathways of 10 or more, 20 or more, or 30 or more locations across the caudate nucleus, the caudate head, the caudate body, the frontal lobe, and/or the auditory cortex regions of the brain.

In some cases, magnetic stimulation is generated by at least one of a Low Field Magnetic Stimulator (LFMS), a Magnetic Resonance Imager (MRI), a Transcranial Magnetic Stimulator (TMS), a Neuro-EEG synchronization Therapy device, or a combination thereof.

In some cases, the treatment comprises stimulation of 1 or more locations, 5 or more locations, 10 or more locations, 15 or more locations, 20 or more locations, 25 or more locations, 30 or more locations, 35 or more locations, 40 or more locations, 45 or more locations, 50 or more locations, 55 or more locations, or 60 or more locations in the caudate nucleus, the caudate head, the caudate body, the frontal lobe, and/or the auditory cortex regions of the brain. In some cases, the treatment comprises stimulation in a combination of the caudate nucleus, the caudate head, the caudate body, the frontal lobe, and/or the auditory cortex regions of the brain. In some cases, the treatment comprises stimulation of 1 or more, 5 or more, 10 or more, 15 or more, 20 of more 25 or more, or 30 or more locations in the caudate nucleus region of the brain. In some cases, the treatment comprises stimulation of 1 or more, 5 or more, 10 or more, 15 or more, 20 of more 25 or more, or 30 or more locations in the caudate body region of the brain. In some cases, the treatment comprises stimulation of 1 or more, 5 or more, 10 or more, 15 or more, 20 of more 25 or more, or 30 or more locations in the caudate head region of the brain.

In some cases, the electrical stimulation comprises deep brain stimulation and/or macrostimulation. In some cases, the method further comprises positioning electrodes at 1 or more locations, 5 or more locations, 10 or more locations, 15 or more locations, 20 or more locations, 25 or more locations, 30 or more locations, 35 or more locations, 40 or more locations, 45 or more locations, 50 or more locations, 55 or more locations, or 60 or more locations of the caudate nucleus, the caudate head, the caudate body, the frontal lobe, and/or the auditory cortex regions of the brain. In some cases, the method further comprises positioning electrodes at 5 locations in the caudate nucleus. In some cases, the method further comprises positioning electrodes at 1 location in the caudate head, and at 4 locations in the caudate head.

### MEGI

Aspects of the present methods include performing magnetoencephalographic imaging (MEGI) of the brain in the subject to acquire MEGI functional connectivity data. With MEGI, a spectral profile of rhythmic neural activity can be used to describe modulations more accurately in resting-state networks. Known signal source analysis methods permit reconstruction of evoked activations from MEGI data. In some cases, MEGI functional connectivity data is resting-state MEGI functional connectivity data.

In some cases, performing MEGI of at least one region of the brain comprises collecting MEGI functional activity data. In some cases, MEGI functional connectivity data is acquired using an MEGI device. In some cases, the MEGI device is a resting-state MEGI device.

Aspects of the present methods include acquiring MEGI functional connectivity data in at least one region of the brain. In some cases, the MEGI functional connectivity data is acquired in at least two regions of the brain. In some cases, the MEGI functional connectivity data is acquired in at least three, at least four, at least five, at least 6, at least seven, at least eight, at least nine, or at least ten regions of the brain. In some cases, the functional connectivity data is acquired in the entire brain.

Functional connectivity data may be acquired from any suitable brain region. Suitable brain regions include, without limitation, caudate dorsal striatum, caudate head, nucleus accumbens, auditory cortex, frontal lobe, thalamus, non-auditory cortex, ventral tegmental area (VTA), prefrontal cortex (PFC), amygdala, substantia nigra, ventral pallidum, globus pallidus, ventral striatum, subthalamic nucleus, anterior caudate putamen, globus pallidus external, anterior supramarginal gyrus, globus pallidus internal, hippocampus, dentate gyrus, cingulate gyrus, entorhinal cortex, olfactory cortex, motor cortex, cerebellum, lateral occipital cortex, and cuneus.

In some cases, acquiring MEGI of the brain includes collecting MEGI signals from the subject for a period of time. In some cases, the MEGI signals include 0.5 kHz, 1 kHz, 1.5 kHz, 2 kHz, 2.5 kHz, 3 kHz, 3.5 kHz, 4 kHz, 4.5 kHz, 5 kHz, 5.5 kHz, 6 kHz, 6.5 kHz, 7 kHz, 7.5 kHz, 8 kHz, 8.5 kHz, 9 kHz, 9.5 kHz, or 10 kHz of MEGI signals. In some cases, the MEGI signals range from 0-5 kHz, 5-10 kHz, 10-15 kHz, 15-20 kHz, 20-25 kHz, or 25-30 kHz of signals. In some cases, the MEGI signals are acquired and/or collected in the alpha frequency range. In some cases, the MEGI signals are acquired in the 8-12 Hz frequency range. In some cases, the period of time for collecting MEGI signals include 1 minute, 2 minutes, 3 minutes, 4 minutes, 5 minutes, 6 minutes, 7 minutes, 8 minutes, 9 minutes, or 10 minutes. In some cases, the period of time for collecting MEGI signals include 1-5 minutes, 5-10 minutes, 10-15 minutes, 15-20 minutes, 20-25 minutes or 25-30 minutes.

In some cases, performing MEGI of the brain is performed with the subject's eyes closed. In some cases, performing MEGI of the brain is performed with the subject's eyes open. In some cases, performing MEGI of the brain is performed with the subject in a supine position. In some cases, performing MEGI of the brain is performed with the subject's eyes open without any instruction to perform an explicit task requiring active engagement during data acquisition.

In some cases, performing MEGI functional connectivity data comprises measuring, recording, and/or collecting time-frequency signals of bihemispheric auditory cortices. In some cases, a three-dimensional (3D) grid of voxels with 2 mm spatial resolution covering the entire brain is created for each subject.

In some cases, acquiring MEGI functional connectivity data comprises collecting MEGI data signals from a plurality of sensors surrounding the brain of the subject. In some cases, the plurality of sensors include an array of MEGI sensors. In some cases, the array of sensors comprise an array of biomagnetometer sensors. In some cases, the array of biomagnetometer sensors measure small changes in immediate magnetic field, wherein the small changes are generated by the brain activity of the subject. In some cases, the array of sensors (e.g. biomagnetometer sensors) are housed in a helmet. In some cases, the array of sensors are evenly distributed over head of the helmet. In some cases, the biomagnetometer is a multi-channel biomagnetometer.

In some cases, acquiring MEGI functional connectivity data includes collecting MEGI signals and fitting the MEGI signal data to a multisphere head model of co-registered structural 3D T1-weight MR scans from the subject.

Aspects of the present methods include assessing the MEGI functional connectivity data. In some cases, assessing includes assessing the MEGI signals recorded from a MEGI device. In some cases, the MEGI device is a resting-state MEGI device.

In some cases, assessing MEGI functional connectivity comprises assessing MEGI signals and reconstructing three-dimensional MEGI images using a three-dimensional tomographic map.

In some cases, MEGI functional connectivity data patterns are assessed and/or analyzed by defining seed regions using functional brain organization maps. In some cases, the seed regions are defined using stereotactic coordinates of a three-dimensional space in the brain. In some cases, the seed regions are defined using a three-dimensional statistical map.

In some cases, assessing and/or analyzing MEGI functional connectivity data comprises extracting connectivity values (i.e. correlation and/or coherence coefficients) from three-dimensional connectivity maps.

In some cases, MEGI functional connectivity data of the present methods comprises a plurality of images. In some cases, the plurality of images are constructed from the MEGI signals from the subject. In some cases, acquiring the MEGI signals from the subject comprises reconstructing the MEGI signals into three-dimensional (3D) images. In some cases, the 3D images are functional and structural 3D images. In some cases, a reconstruction algorithm is used to reconstruct the electromagnetic neural activity at each brain voxel from the MEGI signal. In some cases, alignment of structural and functional images is conducted by marking at least 1, at least 2, or at least 3 prominent anatomical points on the subject's head in MR images of the subject and localizing at least 1, at least 2, or at least 3 or more fiducials attached to the same points before and after each MEGI scan. In some cases, the following procedures are deployed: 1) fiducials are placed at the left and right periauricular points and at the nasion using localizing sensors in MEGI device, and 2) identical positions are marked on the subject's T1-weighted anatomical MRI for alignment with the MEGI position sensors. In some cases, alignment of structural and functional images is conducted by marking at least 3 or more prominent anatomical points on the subject's head in MR images and localizing 3 or more fiducials attached to the same points before and after each MEGI scan. Non-limiting examples of constructing MEGI signals into three dimensional images of the electrophysiological activity within the brain is described in U.S. Pat. No. 6,697,660, which is hereby incorporated by reference in its entirety.

In some cases, acquiring MEGI functional connectivity data comprises acquiring MEGI image data from the subject's brain. In some cases, the imaging data is reconstructed into three-dimensional (3D) images. Non-limiting programs that may be used to reconstruct 3D images include Matlab®, Voloom (microDimensions, Munich, Germany), Imaris, Image-Pro Premier 3D (Media Cybernetics, Rockville, Md., USA), or any available 3D reconstruction software. In some cases, acquiring MEGI functional connectivity data comprises reconstructing, from a plurality of acquired MR image, 3D MR images of the subject's brain by starting from a seed location within the brain and building the model outward to the surface of the brain.

Aspects of the present methods include assessing the MEGI functional connectivity data of the frontal cortex region of the brain. In some cases, assessing the MEGI functional connectivity data comprises assessing the three-dimensional images reconstructed from the functional connectivity data. In some cases, assessing the MEGI functional connectivity data comprises assessing the MEGI signals collected from the MEGI device. In some cases, assessing the MEGI functional connectivity data includes assessing the hyposynchrony in the frontal cortex of the brain. In some cases, assessing the MEGI functional data comprises assessing the global connectivity of the frontal cortex of the brain with the rest of the brain. In some cases, assessing the MEGI functional connectivity data comprises assessing patterns of hypoconnectivity and/or hyperconnectivity with MEGI functional connectivity data of an individual or subject without Tinnitus. In some cases, assessing patterns of functional connectivity of a region of the brain and comparing the functional connectivity to functional connectivity distributions with the rest of the brain. In some cases, the frontal cortex hyposynchrony magnitude is correlated with Tinnitus severity level.

In some cases, assessing the MEGI functional connectivity data comprises assessing shifts in MEGI bandwidth frequencies in the frontal cortex. In some cases, decreased MEGI functional connectivity comprises decreased MEGI alpha-band activity. In some cases, decreased MEGI functional connectivity comprises decreased MEGI alpha-band activity ranging from 7-8 Hz, 8-9 Hz, 9-10 Hz, 10-11 Hz, 11-12 Hz, or 12-13 Hz. In some cases, decreased MEGI functional connectivity comprises decreased MEGI alpha-band activity ranging from 8-12 Hz. In some cases, assessing the MEGI functional connectivity comprises assessing patterns of abnormal functional connectivity of the frontal cortex of the brain. In some cases, assessing the MEGI functional connectivity comprises assessing patterns of abnormal functional connectivity of the left and/or right left and/or right superior frontal gyrus of the brain. In some cases, assessing and/or determining the MEGI functional connectivity data comprises comparing patterns of functional connectivity in the brain of the subject with a database that includes one or more patterns of functional connectivity in the brain associated with subjects without Tinnitus and/or hearing loss, subjects with mild Tinnitus, subjects with moderate Tinnitus, subjects with severe Tinnitus, subjects with acute or chronic hearing loss, subjects with single-sided hearing loss, subjects with Tinnitus and single-sided hearing loss, subjects with Tinnitus and hearing loss, and/or a combination thereof. Thresholds are determined by statistical analyses of subjects without Tinnitus for each pairwise connectivity comparison. Subjects will be compared against a null distribution in which statistical significance (p<0.05, corrected for multiple comparisons) acts as a threshold.

In some cases, the method comprises assessing the MEGI functional connectivity data of the frontal cortex region of the brain. In some cases, assessing the MEGI functional connectivity data comprises assessing shifts in functional MEGI bandwidth frequencies. In some cases, assessing the MEGI functional connectivity data comprises comparing shifts in functional MEGI bandwidth frequencies in the brain of the subject with a database that includes MEGI bandwidth frequencies in the brain associated with subjects without Tinnitus and/or hearing loss, subjects with mild Tinnitus, subjects with moderate Tinnitus, subjects with severe Tinnitus, subjects with acute or chronic hearing loss, subjects with single-sided hearing loss, subjects with Tinnitus and single-sided hearing loss, subjects with Tinnitus and hearing loss, and/or a combination thereof. In some cases, assessing MEGI functional connectivity of the brain comprises examining time-frequency activation patterns in the brain of the subject. In some cases, assessing MEGI functional connectivity of the brain comprises comparing the time-frequency activation patterns in the brain of the subject with a database that includes time-frequency activation patterns in the brain of subjects with without Tinnitus and/or hearing loss, subjects with mild Tinnitus, subjects with moderate Tinnitus, subjects with severe Tinnitus, subjects with acute or chronic hearing loss, subjects with asymmetric hearing loss, subjects with Tinnitus and asynnetric hearing loss, subjects with Tinnitus and hearing loss, and/or a combination thereof.

Aspects of the present methods further include recording and/or measuring auditory evoked field (AEF) peaks in the subject in response to a pure-tone stimulus to determine spatiotemporal auditory cortical activity in the subject. In some cases, the AEF peaks are measured using a MEGI device. In some cases, the AEF peaks are measured using a task-based MEGI device. In some cases, the spatiotemporal auditory activity is evoked by the pure-stone stimulus at 0.5 kHz. In some cases, the spatiotemporal auditory activity is evoked by the pure-stone stimulus at 1 kHz. In some cases, the spatiotemporal auditory activity is evoked by the pure-stone stimulus at 1.5 kHz, 2 kHz, 2.5 kHz, 3 kHz, 3.5 kHz, 4 kHz, 4.5 kHz, and/or 5 kHz. In some cases, a pure-tone stimulus is a stimulus signal emitted at a particular human audible frequency. In some cases, the method further comprises instructing the subject to confirm if the subject can hear the pure-tone stimulus signal and produce a behavioral response.

In some cases, the method further comprises measuring the AEF latency in response to the pure-tone stimulus. In some cases, the AEF peaks in the left frontal gyrus of the subject have increased latency in response to the pure-tone stimulus as compared to the AEF latency in response to a pure-tone stimulus of the left frontal gyrus in a subject without Tinnitus.

Aspects of the present disclosure include determining if the fMRI functional connectivity data, the MEGI functional connectivity data, and/or the spatiotemporal auditory cortical activity are above, below, or at a threshold level associated with a positive diagnosis of Tinnitus.

In some cases, a positive diagnosis of Tinnitus comprises increased fMRI functional connectivity between the caudate nucleus and auditory cortex as compared to the threshold level, decreased MEGI functional connectivity in the frontal cortex as compared to the threshold level, and/or delayed latency of the AEF peaks in response to the pure-tone stimulus as compared to the threshold level.

In some cases, a positive diagnosis of Tinnitus comprises increased fMRI functional connectivity between the caudate nucleus and auditory cortex as compared to the threshold level.

In some cases, a positive diagnosis of Tinnitus comprises decreased MEGI functional connectivity in the frontal cortex as compared to the threshold level. In some cases, frontal cortex hyposynchrony magnitude is correlated with Tinnitus severity level. In some cases, functional connectivity strength of the left superior frontal gyrus is associated with Tinnitus severity.

In some cases, a positive diagnosis of Tinnitus comprises delayed latency of the AEF peaks in response to the pure-tone stimulus as compared to the threshold level. In some cases, the left frontal gyrus is correlated with Tinnitus distress magnitude and increased latency of the peak M100 response to a 1 kHz tone.

Aspects of the present disclosure include determining if the fMRI functional connectivity data, the MEGI functional connectivity data, and/or the AEF peaks comprise patterns of abnormal functional connectivity and spatiotemporal auditory cortical activity latency.

In some cases, patterns of abnormal connectivity and spatiotemporal auditory cortical activity latency comprise i) increased fMRI functional connectivity between the caudate nucleus and auditory cortex as compared to normal functional connectivity patterns, decreased MEGI functional connectivity in the frontal cortex as compared to normal functional connectivity patterns, and/or delayed latency of the AEF peaks in response to a pure-tone stimulus that are above, below, or at a threshold level associated with a positive diagnosis of Tinnitus.

Aspects of the present methods include determining if the MEGI functional connectivity data is above, below, or at a reference level associated with at least one or more pathology profiles of Tinnitus. In some cases, at least one pathology profile of Tinnitus comprises modulated MEGI functional connectivity in the frontal lobe as compared to the reference level. In some cases, modulated MEGI functional connectivity comprises an increase in functional connectivity as compared to the reference level. In some cases, modulated MEGI functional connectivity comprises a decrease and/or reduction in functional connectivity as compared to the reference level. In some cases, modulated MEGI functional connectivity comprises increased functional connectivity in the frontal lobe region of the brain as compared to the reference level. In some cases, modulated MEGI functional connectivity comprises decreased functional connectivity in the frontal lobe region of the brain as compared to the reference level. In some cases, determining comprises determining shifts in MEGI bandwidth frequencies in the frontal cortex as compared to MEGI bandwidth frequencies associated with the one or more pathology profiles of Tinnitus. In some cases, at least one pathology profile of Tinnitus comprises decreased MEGI functional connectivity in the frontal cortex as compared to the reference level. In some cases, frontal cortex hyposynchrony magnitude is correlated with Tinnitus severity level. In some cases, functional connectivity strength of the left superior frontal gyrus is associated with Tinnitus severity.

In some cases, the at least one pathology profile comprises at least two pathology profiles. In some cases, the at least one pathology profile comprises at least three pathology profiles. In some cases, the at least one pathology profile comprises at least two, at least three, at least four, at least five, at least six, at least seven, at least eight, at least nine, or at least ten pathology profiles.

In some cases, the reference level comprises one reference level. In some cases, the reference level comprises two reference levels. In some cases, the reference level comprises three reference levels. In some cases, the reference level comprises at least one, at least two, at least three, at least four, at least five, at least six, at least seven, at least eight, at least nine, or at least ten reference levels. In some cases, the reference level comprises a first, a second, a third, a fourth, a fifth, a sixth, a seventh, an eighth, a ninth, and/or a tenth reference level.

In some cases, at least one pathology profile of Tinnitus comprises modulated fMRI functional connectivity between the caudate nucleus and the frontal lobe regions of the brain as compared to a first reference level; modulated fMRI functional connectivity between the caudate nucleus and the auditory cortex regions of the brain as compared to a second reference level; and/or modulated MEGI functional connectivity in the frontal lobe as compared to a third reference level. In some cases, at least one pathology profile further comprises delayed latency of the AEF peaks in response to the pure-tone stimulus as compared to a fourth reference level. In some cases, the first, the second, and the third reference level are the same. In some cases, the first, the second, and the third reference level are different. In some cases, the first, the second, the third, and the fourth reference level are the same. In some cases, the first, the second, the third, and the fourth reference level are different.

In some cases, the strength and/or magnitude of functional connectivity between the caudate nucleus and non-auditory structures is correlated with tinnitus severity domains. In some cases, functional connectivity strength between the caudate nucleus and the cuneus is correlated with relaxation difficulty attributed to Tinnitus. In some cases, functional connectivity strength between the caudate nucleus and the superior lateral occipital cortex is correlated with control difficulty attributed to tinnitus. In some cases, the functional connectivity strength between the caudate nucleus and anterior supramarginal gyrus is correlated with control difficulty attributed to tinnitus.

In some cases, the one or more pathology profiles comprises modulated MEGI functional connectivity strength and/or magnitude is correlated with Tinnitus severity level. In some cases, the one or more pathology profiles comprises shifts in MEGI bandwidth frequencies in the frontal cortex as compared to MEGI bandwidth frequencies. In some cases, the one or more pathology profiles comprises reduced MEGI alpha-band activity.

In some cases, assessing and/or determining the MEGI functional connectivity comprises comparing patterns of functional connectivity in the brain of the subject with a database that includes one or more patterns of functional connectivity in the brain associated with subjects without Tinnitus and/or hearing loss, subjects with mild Tinnitus, subjects with moderate Tinnitus, subjects with severe Tinnitus, subjects with acute or chronic hearing impairment, subjects with symmetric and/or asymmetric hearing impairment, subjects with Tinnitus and symmetric and/or asymmetric hearing impairment, subjects with Tinnitus and hearing impairment, and/or a combination thereof. In some cases, hearing impairment comprises acute and/or chronic hearing loss; symmetric and/or asymmetric hearing loss; and/or a combination thereof. In some cases, Tinnitus can include acute or chronic tinnitus.

In some cases, assessing and/or determining the MEGI functional connectivity comprises providing a database that provides one or more pathology profiles associated with Tinnitus or hearing impairment. In some cases, the one or more pathology profiles comprises patterns of functional connectivity associated with subjects without Tinnitus and/or hearing loss, subjects with mild Tinnitus, subjects with moderate Tinnitus, subjects with severe Tinnitus, subjects with acute or chronic hearing impairment, subjects with symmetric and/or asymmetric hearing impairment, subjects with Tinnitus and symmetric and/or asymmetric hearing impairment, subjects with Tinnitus and hearing impairment, and/or a combination thereof. In some cases, hearing impairment comprises acute and/or chronic hearing loss; symmetric and/or asymmetric hearing loss; and/or a combination thereof. In some cases, Tinnitus can include acute or chronic tinnitus. In some cases, hearing impairment can include acute or chronic hearing loss, and/or symmetric or asymmetric hearing loss.

Aspects of the present methods include a method of detecting Tinnitus in a subject, the method comprising acquiring and assessing MEGI functional connectivity data to determine if the MEGI functional connectivity data is above, below, or at a reference level associated with one or more pathology profiles of Tinnitus. In some cases, the method further comprises acquiring and assessing MEGI functional connectivity data is above, below, or at a reference level associated with one or more pathology profiles of Tinnitus.

Aspects of the present methods include a method of analyzing images of the brain. In some cases, the method includes providing a database, using logistic regression algorithms, that comprises one or more pathology profiles associated with Tinnitus or hearing impairment. In some cases, the method comprises receiving a plurality of fMRI images and/or functional MEGI images of at least one region of the brain. In some cases, the method comprises analyzing the plurality of fMRI and/or MEGI images of at least one region of the brain. In some cases, the method comprises analyzing the plurality of fMRI and/or MEGI images to obtain fMRI and MEGI functional connectivity data. In some cases, the method comprises comparing the fMRI and/or MEGI functional connectivity data from the fMRI or MEGI images with the one or more pathology profiles of step associated with Tinnitus or hearing impairment. In some cases, hearing impairment includes acute or chronic hearing loss; symmetric or asymmetric hearing loss; or a combination thereof. In some cases, the one or more pathology profiles is derived from a plurality of fMRI or MEGI images of one or more subjects having one or more pathology profiles. In some cases, the plurality of fMRI and/or MEGI images are three dimensional images.

In some cases, the method comprises receiving AEF data in response to a pure-tone stimulus. In some cases, the AEF data comprises AEF peaks corresponding to spatiotemporal auditory cortical activity. In some cases, the database further comprises AEF data associated with the one or more pathology profiles. In some cases, the method comprises comparing latency of the AEF peaks in response to the pure-tone stimulus with the AEF data associated with the one or more pathology profiles.

Aspects of the present methods include method of analyzing fMRI signals and/or MEGI signals of the brain. In some cases, the method comprises providing a database, using logistic regression algorithms, that comprises one or more pathology profiles associated with Tinnitus or hearing impairment. In some cases, the method comprises receiving fMRI signals and/or MEGI signals from at least one region of the brain. In some cases, the method comprises analyzing the plurality of fMRI and/or MEGI signals to obtain fMRI and/or MEGI functional connectivity data. In some cases, the method comprises comparing the fMRI and/or MEGI functional connectivity data from the fMRI or MEGI signals with the one or more pathology profiles.

Aspects of the present methods include a method of treating or reducing Tinnitus in a subject. In some cases, the method includes a) acquiring magnetoencephalographic imaging (MEGI) functional connectivity data of at least one of the caudate nucleus, the caudate head, the caudate body, the frontal lobe, and the auditory cortex regions of the brain of the subject; b) assessing the MEGI functional connectivity data in at the at least one region of the brain; c) determining if the MEGI functional connectivity data are above, below, or at a reference level associated with at least one or more pathology profiles of Tinnitus, wherein at least one pathology profile of Tinnitus comprises: i) modulated MEGI functional connectivity in the frontal lobe as compared to the reference level; or ii) modulated MEGI functional connectivity in the auditory cortex regions as compared to the reference level; and d) delivering electrical, acoustic, or magnetic stimulation in one or more of the caudate nucleus, the caudate head, the caudate body, the frontal lobe, and the auditory cortex regions of the brain to reduce tinnitus loudness in the individual. In some cases, the MEGI functional connectivity data is above a reference level associated with at least one or more pathology profiles of Tinnitus. In some cases, the MEGI functional connectivity data is below a reference level associated with at least one or more pathology profiles of Tinnitus. In some cases, the MEGI functional connectivity data is at a reference level associated with at least one or more pathology profiles of Tinnitus.

Aspects of the present methods include a method of treating or reducing tinnitus in an individual. In some cases, the method comprises delivering electrical, acoustic, and/or magnetic stimulation to the individual to treat or reduce tinnitus. In some embodiments, the method further comprises treating or reducing the individual with tinnitus by delivering electrical, acoustic, and/or magnetic signals to the individual. In some embodiments, the stimulation is synchronized stimulation. In some embodiments, the stimulation is pulsatile stimulation. In some cases, the treatment is acoustic stimulation. In some cases, the treatment is electrical stimulation. In some cases, the stimulation is macrostimulation. In some cases, the stimulation is magnetic stimulation. In some cases, the acoustic stimulation utilizes sound wave cancellation techniques. Non-limiting examples of electrical, acoustic, or magnetic stimulation treatments of Tinnitus can be found in U.S. Pat. Nos.: 6,210,321, 9,649,502, 10,265,527, 8,934,967, 6,610,019, and 9,242,067, which are hereby incorporated by reference in their entirety.

In some cases, performing stimulation comprises delivering one or more synchronized stimulations to the at least one or more of the caudate nucleus, the caudate head, the caudate body, the frontal lobe, and the auditory cortex regions of the brain. In some cases, at least one synchronized stimulation comprises stimulation of multiple non-auditory pathways of 10 or more, 20 or more, or 30 or more locations across the caudate nucleus, the caudate head, the caudate body, the frontal lobe, and/or the auditory cortex regions of the brain.

In some cases, magnetic stimulation is generated by at least one of a Low Field Magnetic Stimulator (LFMS), a Magnetic Resonance Imager (MRI), a Transcranial Magnetic Stimulator (TMS), a Neuro-EEG synchronization Therapy device, or a combination thereof.

In some cases, the treatment comprises stimulation of 1 or more locations, 5 or more locations, 10 or more locations, 15 or more locations, 20 or more locations, 25 or more locations, 30 or more locations, 35 or more locations, 40 or more locations, 45 or more locations, 50 or more locations, 55 or more locations, or 60 or more locations in the caudate nucleus, the caudate head, the caudate body, the frontal lobe, and/or the auditory cortex regions of the brain. In some cases, the treatment comprises stimulation in a combination of the caudate nucleus, the caudate head, the caudate body, the frontal lobe, and/or the auditory cortex regions of the brain. In some cases, the treatment comprises stimulation of 1 or more, 5 or more, 10 or more, 15 or more, 20 of more 25 or more, or 30 or more locations in the caudate nucleus region of the brain. In some cases, the treatment comprises stimulation of 1 or more, 5 or more, 10 or more, 15 or more, 20 of more 25 or more, or 30 or more locations in the caudate body region of the brain. In some cases, the treatment comprises stimulation of 1 or more, 5 or more, 10 or more, 15 or more, 20 of more 25 or more, or 30 or more locations in the caudate head region of the brain.

In some cases, the electrical stimulation comprises deep brain stimulation and/or macrostimulation. In some cases, the method further comprises positioning electrodes at 1 or more locations, 5 or more locations, 10 or more locations, 15 or more locations, 20 or more locations, 25 or more locations, 30 or more locations, 35 or more locations, 40 or more locations, 45 or more locations, 50 or more locations, 55 or more locations, or 60 or more locations of the caudate nucleus, the caudate head, the caudate body, the frontal lobe, and/or the auditory cortex regions of the brain. In some cases, the method further comprises positioning electrodes at 5 locations in the caudate nucleus. In some cases, the method further comprises positioning electrodes at 1 location in the caudate head, and at 4 locations in the caudate head.

### Systems

The present disclosure includes systems for determining the presence of Tinnitus in a subject. Also provided are systems configured for performing the disclosed methods and computer readable medium storing instructions for performing steps of the disclosed methods.

In some embodiments, the system is an automated system. In some embodiments, the system is multimodal neuroimaging system.

In some embodiments, the comprises: a) a functional magnetic resonance imaging (fMRI) device and/or a magnetoencephalographic imaging (MEGI) device; b) at least one memory storage medium configured to store functional connectivity data of the brain of the subject received from the fMRI and/or MEGI device; e) at least one processor operably coupled to the at least one memory storage medium, the at least one processor being configured to: i) process fMRI data and/or MEGI data recorded from at least one region of the brain in an individual, thereby generating fMRI functional connectivity data and/or MEGI functional connectivity data; ii) analyze fMRI and/or MEGI functional connectivity data, iii) prune, using logistic regression algorithms, the fMRI functional connectivity data and/or the MEGI functional connectivity data; iv) compare the fMRI and/or MEGI functional connectivity data obtained in step iii with functional connectivity data obtained from a database comprising one or more pathology profiles associated with Tinnitus; and v) determine if the individual has Tinnitus based on the data obtained in step iv.

In some embodiments, the system includes a fMRI device. Non-limiting examples of components of an fMRI include an operator workstation, a display, one or more input devices and/or a computer, and a processor. In some cases, the fMRI device includes a 32-channel receive-only array with a volume transmit head coil on a FMRI device. In some cases, the processor may include a commercially available programmable machine running a commercially available operating system. The operator workstation provides the operator interface that enables scan prescriptions to be entered into the fMRI device. In general, the operator workstation may be coupled to one or more, two or more, three or more, or four or more servers. Non-limiting examples of servers include a pulse sequence server; a data acquisition server; a data processing server; and a data store server. The operator workstation and each server are connected to communicate with each other. For example, the servers may be connected via a communication system, which may include any suitable network connection, whether wired, wireless, or a combination of both. As an example, the communication system may include both proprietary or dedicated networks, as well as open networks, such as the internet. Suitable fMRI devices are described in, e.g., U.S. Pat. No. 8,834,546; 9,662,039; U.S. Application Publication No. 2016/0270723; and PCT Application Nos. PCT/US2016/043179; PCT/US2016/064250; and PCT/US2016/049508, each of the disclosures of which are incorporated herein by reference.

Among the processing tasks for operating the fMRI, the at least one processor may also be configured to receive a population atlas, variation map and time-series fMRI data, wherein the received time-series fMRI data may be pre-processed, and/or may undergo any number of further processing steps using the at least one processor. In some aspects, the at least one processor may be capable of performing computations using time-series signals derived from time-series fMRI data. For example, the at least one processor may be capable of combining any time-series signals associated with brain locations assigned to specific functional networks, or may be capable of correlating time-series signals in relation to any functional connectivity networks. Specifically, such iterative process, as will be described, may be guided by population information, such as organization and variability in functional networks of a population, as well as individual subject information, such as a signal-to-noise ratio determined from time-series fMRI data acquired from that subject. A suitable example of an fMRI device with at least one processor is described in U.S. Pat. No. 9,662,039, the disclosure of which is incorporated herein by reference. Non-limiting examples of fMRI devices include the 0.5 T Paramed Upright MRI scanner, the 1.5 Tesla GE HDxt MRI Scanner, the 3 Tesla GE Discovery MR750 MRI Scanner, the 3 Tesla Philips Achieva MRI Scanner, the 3 Tesla Philips Ingenia Wide Bore MRI Scanner, the Siemens fMRI, and the 7 Tesla Philips Achieva MRI Scanner.

In some embodiments, the system includes a MEGI device. MEGI devices measures magnetic fields produced by the brain. Commercially available MEGI scanners sense and map the minute magnetic fields associated with the electric voltages and currents generated by large groups of firing neurons within the brain, and construct a three-dimensional map of detected neural activity. Non-limiting MEGI devices include the CTF MEGI scanner and the 4D Neuroimaging MEGI.

In some embodiments, the MEGI signal is recorded by a MEGI sensor array. In some cases, the array of sensors comprise an array of biomagnetometer sensors. In some cases, the array of sensors comprise an array of biomagnetometer sensors ranging from 100-125 biomagnetometer sensors, 125-150 biomagnetometer sensors, 150-175 biomagnetometer sensors, 200-225 biomagnetometer sensors, 225-250 biomagnetometer sensors, 250-275 biomagnetometer sensors, 275-300 biomagnetometer sensors, 300-325 biomagnetometer sensors, 325-350 biomagnetometer sensors, 350-375 biomagnetometer sensors, or 375-400 biomagnetometer sensors. In some cases, the array of sensors comprise an array of 200 biomagnetometer sensors, 205 biomagnetometer sensors, 210 biomagnetometer sensors, 215 biomagnetometer sensors, 220 biomagnetometer sensors, 225 biomagnetometer sensors, 230 biomagnetometer sensors, 235 biomagnetometer sensors, 240 biomagnetometer sensors, 245 biomagnetometer sensors, 250 biomagnetometer sensors, 255 biomagnetometer sensors, 260 biomagnetometer sensors, 265 biomagnetometer sensors, 270 biomagnetometer sensors, 275 biomagnetometer sensors, 280 biomagnetometer sensors, 285 biomagnetometer sensors, 290 biomagnetometer sensors, 295 biomagnetometer sensors, or 300 biomagnetometer sensors. In some cases, the array of biomagnetometer sensors measure small changes in immediate magnetic field, wherein the small changes are generated by the brain activity of the subject. In some cases, the array of sensors (e.g. biomagnetometer sensors) are housed in a helmet. In some cases, the array of sensors are evenly distributed over head of the helmet.

Aspects of the present system include a pure-tone stimulus. In some embodiments, a pure tone stimulus will be sampled at 1 kHz with a MEGI sensor array of 275 axial magnetometers that span the whole scalp surface of the subject. In some cases, the magnetometers are biomagnetometers.

Aspects of the present system include at least one memory storage medium configured to store functional connectivity data of the brain of the subject received from the fMRI and MEGI device.

Aspects of the present system include at least one processor operably coupled to the at least one memory storage medium. In some embodiments, the at least one processor is configured to record fMRI (e.g. resting-state fMRI) functional connectivity of at least one region of the brain in an individual, thereby generating fMRI functional connectivity data for at least one region of the brain. In some embodiments, the at least one processor is configured to record a MEGI (e.g. resting-state MEGI) functional connectivity data for a region of the brain, thereby generating MEGI functional connectivity data. In some embodiments, the at least one processor is configured to record AEF peaks in response to the pure-tone stimulus. In some embodiments, the at least one processor is at least two processors, at least three processors, at least four processors, at least five processors, at least six processors, at least seven processors, at least eight processors, at least nine processors, or at least ten processors. In some embodiments, the processor that is configured to record the fMRI functional connectivity data is the same as the processor that is configured to record the MEGI functional connectivity data and/or the AEF peaks in response to the pure-tone stimulus. In some embodiments, the processor that is configured to record the fMRI functional connectivity data is different than the processor that is configured to record the MEGI functional connectivity data and/or the AEF peaks in response to the pure-tone stimulus. In some embodiments, the processor that is configured to record the MEGI functional connectivity data is the same as the processor that is configured to record the AEF peaks in response to the pure-tone stimulus. In some embodiments, the processor that is configured to record the MEGI functional connectivity data is the different than the processor that is configured to record the AEF peaks in response to the pure-tone stimulus.

In some embodiments, the processor is configured to identify latencies of the AEF peaks derived from the auditory cortex of the subject in response to the pure-tone stimulus. AEF is a form neural activity that is induced by an auditory stimulus. At 100 ms after stimulus onset occurs, the change in the magnetic field over auditory cortex in response to the 100-msec latency range is termed “M100”. The M100 wave corresponds to the N1 peak of the auditory long latency response (ALR) potential.

By “data processing unit” or “processor”, as used herein, is meant any hardware and/or software combination that will perform he functions required of it. For example, any data processing unit herein may be a programmable digital microprocessor such as available in the form of an electronic controller, mainframe, server or personal computer (desktop or portable). Where the data processing unit is programmable, suitable programming can be communicated from a remote location to the data processing unit, or previously saved in a computer program product (such as a portable or fixed computer readable storage medium, whether magnetic, optical or solid-state device based).

Substantially any circuitry can be configured to a functional arrangement within the devices and systems for performing the methods disclosed herein. The hardware architecture of such circuitry, including e.g., a specifically configured computer, is well known by a person skilled in the art, and can comprise hardware components including one or more processors (CPU), a random-access memory (RAM), a read-only memory (ROM), an internal or external data storage medium (e.g., hard disk drive). Such circuitry can also comprise one or more graphic boards for processing and outputting graphical information to display means. The above components can be suitably interconnected via a bus within the circuitry, e.g., inside a specific-use computer. The circuitry can further comprise suitable interfaces for communicating with general-purpose external components such as a monitor, keyboard, mouse, network, etc. In some embodiments, the circuitry can be capable of parallel processing or can be part of a network configured for parallel or distributive computing to increase the processing power for the present methods and programs. In some embodiments, the program code read out from the storage medium can be written into a memory provided in an expanded board inserted in the circuitry, or an expanded unit connected to the circuitry, and a CPU or the like provided in the expanded board or expanded unit can actually perform a part or all of the operations according to the instructions of the programming, so as to accomplish the functions described.

The systems of the present disclosure may further include a “memory” that is capable of storing information such that it is accessible and retrievable at a later date by a computer. Any convenient data storage structure may be chosen, based on the means used to access the stored information. In certain aspects, the information may be stored in a “permanent memory” (i.e. memory that is not erased by termination of the electrical supply to a computer or processor) or “non-permanent memory”. Computer hard-drive, CD-ROM, floppy disk, portable flash drive and DVD are all examples of permanent memory. Random Access Memory (RAM) is an example of non-permanent memory. A file in permanent memory may be editable and re-writable.

In addition to the components of the devices and systems of the present disclosure, e.g., as described above, systems of the disclosure may include a number of additional components, such as data output devices, e.g., monitors and/or speakers, data input devices, e.g., interface ports, keyboards, etc., fluid handling components, slide handling components, power sources, etc.

### Regression Algorithms

Aspects of the present systems include at least one processor operably coupled to the at least one memory storage medium, the at least one processor being configured to prune, using logistic regression algorithms, the fMRI functional connectivity data and the MEGI functional connectivity data. In some embodiments, the logistic regression algorithms use both fMRI and MEGI data and neuropsychological and audiological clinical data to determine if the individual has Tinnitus, with or without acute or chronic hearing loss. In some embodiments, the logistic regression algorithm includes logistic regression models. In some embodiments, the regression models deploy variants of relevance vector machines to perform pruning for diagnostic tool refinement. In some embodiments, the logistic regression algorithm is a sparse Bayesian logistic regression algorithm.

In some embodiments, pruning includes applying automatic relevance determination (ARD) and the sparse Bayesian learning (SBL) framework effective algorithms to prune large numbers of irrelevant features leading to a sparse explanatory subset. In some embodiments, ARD is equivalent to performing standard maximum a posteriori (MAP) estimation in a dual space using particular-feature and noise-dependent, non-factorial weighted priors. In some embodiments, the logistic regression algorithm is a linear least squares regression, robust linear regression, support vector machine, k-means clustering, or ridge regression. In some embodiments, the logistic regression algorithms comprise a plurality of logistic regression models. In some embodiments, the logistic regression algorithm is a relevance vector machine that executes automatic feature pruning. Relevance vector machines are Bayesian-based machine learning algorithms that use parsimonious solutions for regression and probabilistic classification. In some embodiments, the logistic regression algorithm deploys variants of relevance vector machines to perform pruning. In some embodiments, the machine learning sparse Bayesian logistic regression algorithm is a relevance vector machine that involves no approximation steps and descends a well-defined objective function.

In some embodiments, wherein at least one of the plurality of logistic regression models comprises predictor variables of functional connectivity data. In some embodiments, the at least one or the plurality of logistic regression models comprises predictor variables of functional connectivity data. In such embodiments, the functional connectivity data comprises functional connectivity at each oscillatory frequency. In some embodiments, the functional connectivity at each oscillatory frequency is quantified by averaging the imaginary component of coherence across a plurality of seeds.

In some embodiments, the processor is configured to determine if the individual has Tinnitus, with or without hearing loss based on the logistic regression models and latencies of the AEF peaks derived from the auditory cortex of the subject in response to the pure-tone stimulus.

In some embodiments, the processor is configured to determine if the individual has Tinnitus, with or without hearing loss based on a binomial logistic regression model of functional connectivity between the caudate and auditory cortex of the brain. In some embodiments, the binomial logistic regression model comprises functional connectivity values from bihemispheric caudate connectivity maps extracted from the ipsilateral posterior middle temporal gyrus of the brain. Binomial regressions involve prediction a response (Y) as one of two possible outcomes (e.g. tinnitus or no-tinnitus) related to one or more explanatory variables, such as strength of functional connectivity.

### Computer Readable Medium

The present disclosure includes computer readable medium, including non-transitory computer readable medium, which stores instructions for detecting Tinnitus, hearing impairment, and/or primary progressive aphasia. Aspects of the present disclosure include computer readable medium storing instructions that, when executed by a computing device, cause the computing device to perform one or more of the steps of i) recording fMRI functional connectivity of a brain of an individual, thereby generating fMRI functional connectivity data for at least one region of the brain; ii) recording MEGI functional connectivity data for a region of the brain, thereby generating MEGI functional connectivity data; iii) recording auditory-evoked field (AEF) peaks in response to the pure-tone stimulus; iv) pruning, using logistic regression algorithms, the fMRI functional connectivity data and/or the MEGI functional connectivity data; v) identifying latencies of the AEF peaks derived from the auditory cortex of the subject in response to the pure-tone stimulus; and/or vi) determining if the individual has Tinnitus and/or hearing impairment based on the data obtained from step iv and v. In some embodiments, the computing device is a processor or a data processing unit.

In some embodiments, aspects of the present disclosure include computer readable medium storing instructions that, when executed by a computing device, cause the computing device to perform one or more of the steps of i) recording resting-state fMRI functional connectivity of a brain of an individual, thereby generating fMRI functional connectivity data for at least one region of the brain; and ii) determining if the individual has Tinnitus with or without hearing impairment based on a binomial logistic regression model of functional connectivity between the caudate and auditory cortex of the brain, wherein the binomial logistic regression model comprises functional connectivity values from bihemispheric caudate connectivity maps extracted from the ipsilateral posterior middle temporal gyrus of the brain.

The devices and systems of the present disclosure may further include a “memory” that is capable of storing information such that it is accessible and retrievable at a later date by a computer. Any convenient data storage structure may be chosen, based on the means used to access the stored information. In certain aspects, the information may be stored in a permanent memory (i.e., memory that is not erased by termination of the electrical supply to a computer or processor) or non-permanent memory. Computer hard-drive, CD-ROM, floppy disk, portable flash drive and DVD are all examples of permanent memory. Random Access Memory (RAM) is an example of non-permanent memory. A file in permanent memory may be editable and re-writable.

Substantially any circuitry can be configured to a functional arrangement within the devices and systems for performing the methods disclosed herein. The hardware architecture of such circuitry, including e.g., a specifically configured computer, is well known by a person skilled in the art, and can comprise hardware components including one or more processors (CPU), a random-access memory (RAM), a read-only memory (ROM), an internal or external data storage medium (e.g., hard disk drive). Such circuitry can also comprise one or more graphic boards for processing and outputting graphical information to display means. The above components can be suitably interconnected via a bus within the circuitry, e.g., inside a specific-use computer. The circuitry can further comprise suitable interfaces for communicating with general-purpose external components such as a monitor, keyboard, mouse, network, etc. In some embodiments, the circuitry can be capable of parallel processing or can be part of a network configured for parallel or distributive computing to increase the processing power for the present methods and programs. In some embodiments, the program code read out from the storage medium can be written into a memory provided in an expanded board inserted in the circuitry, or an expanded unit connected to the circuitry, and a CPU or the like provided in the expanded board or expanded unit can actually perform a part or all of the operations according to the instructions of the programming, so as to accomplish the functions described.

In addition to the components of the devices and systems of the present disclosure, e.g., as described above, systems of the disclosure may include a number of additional components, such as data output devices, e.g., monitors and/or speakers, data input devices, e.g., interface ports, keyboards, etc., actuatable components, power sources, etc.

As summarized above, also provided by the present disclosure are computer-readable media, e.g., which find use in practicing the methods of the present disclosure.

The present disclosure includes computer readable medium, including non-transitory computer readable medium, which stores instructions for methods described herein. Aspects of the present include computer readable medium storing instructions that, when executed by a computing device (e.g., processor of a computing device), cause the computing device to perform one or more steps of a method as described herein. According to certain embodiments, a computer readable medium may include instructions for recording resting-state fMRI functional connectivity data of a brain of an individual, recording a resting-state MEGI functional connectivity data of a brain, recording auditory evoked field peaks in response to a pure-tone stimulus, prune, using machine learning sparse logistic regression algorithms, the fMRI functional connectivity data and the MEGI functional connectivity data, identify latencies of the AEF peaks derived from the auditory cortex of the subject in response to the pure-tone stimulus, and/or determine if the individual has Tinnitus with or without hearing impairment.

Aspects of the present disclosure include a non-transitory computer-readable memory medium comprising instructions that when executed cause a processor to: i) process fMRI data or MEGI data recorded from at least one region of the brain in an individual, thereby generating fMRI functional connectivity data or MEGI functional connectivity data; ii) analyze fMRI or MEGI functional connectivity data, iii) prune, using logistic regression algorithms, the fMRI functional connectivity data or the MEGI functional connectivity data; iv) compare the fMRI or MEGI functional connectivity data obtained in step iii with functional connectivity data obtained from a database comprising one or more pathology profiles associated with Tinnitus; and v) determine if the individual has Tinnitus based on the data obtained in step iv.

Aspects of the present disclosure include a non-transitory computer-readable memory medium comprising instructions that when executed cause a processor to: i) process fMRI data recorded from at least one region of the brain in an individual, thereby generating fMRI functional connectivity data; ii) analyze fMRI functional connectivity data, iii) prune, using logistic regression algorithms, the fMRI functional connectivity data; iv) compare the fMRI functional connectivity data obtained in step iii with functional connectivity data obtained from a database comprising one or more pathology profiles associated with Tinnitus; and v) determine if the individual has Tinnitus based on the data obtained in step iv.

Aspects of the present disclosure include a non-transitory computer-readable memory medium comprising instructions that when executed cause a processor to: i) process MEGI data recorded from at least one region of the brain in an individual, thereby generating MEGI functional connectivity data; ii) analyze MEGI functional connectivity data, iii) prune, using logistic regression algorithms, the MEGI functional connectivity data; iv) compare the MEGI functional connectivity data obtained in step iii with functional connectivity data obtained from a database comprising one or more pathology profiles associated with Tinnitus; and v) determine if the individual has Tinnitus based on the data obtained in iv.

In certain embodiments, instructions in accordance with the methods described herein can be coded onto a computer-readable medium in the form of “programming”, where the term “computer readable medium” as used herein refers to any storage or transmission medium that participates in providing instructions and/or data to a computer for execution and/or processing. Examples of storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer.

The computer-implemented method described herein can be executed using programming that can be written in one or more of any number of computer programming languages. Such languages include, for example, Java (Sun Microsystems, Inc., Santa Clara, Calif.), Visual Basic (Microsoft Corp., Redmond, Wash.), and C++ (AT&T Corp., Bedminster, N.J.), as well as any many others.

In certain aspects, the instructions comprise instructions for converting collected raw data into three dimensional images to acquire functional connectivity data.

### Utility

Subject methods and systems find use in detecting Tinnitus, with or without hearing loss in an individual. Subject methods and systems find use in individuals with Post-traumatic stress disorder (PTSD). PTSD is relatively common among military personnel and Veterans, with a median point prevalence twice that of the general population. PTSD prevalence is about 18% in soldiers exposed to combat and is associated with more troublesome tinnitus. In a cohort of Veterans receiving care at a specialized tinnitus clinic, those with comorbid PTSD showed clinically significant greater severity, poorer sound tolerance capacity, and lower confidence to manage their phantom percept-related problems. Military personnel and Veterans are at risk for noise induced hearing loss and tinnitus. Those with comorbid PTSD are likely to experience greater tinnitus severity.

In addition to PTSD, other behavioral modulators of tinnitus include mood, anxiety, stress and obsessive-compulsive disorder. Mood disorders, principally depression and anxiety, can worsen tinnitus severity and have been reported in tinnitus patients at rates 2-3 times higher than the general population. When modulators of tinnitus, such as stress and anxiety worsen, tinnitus severity often increases in tandem, reinforcing a cycle of heightened auditory phantom distress that drives its modulators to even higher levels of severity. Problematic tinnitus adversely impacts restful sleep, cognitive focus, and psychological wellness, and interferes with sound reception (Tyler et al., 2006; Tyler et al., 2007; Moller, 2016).

Hearing change is often associated with tinnitus modulation. Rapid degradation of audiometric thresholds in idiopathic sudden sensorineural loss, fluctuating hearing loss in Meniere's disease, subacute conductive hearing loss, and sudden mixed conductive and sensorineural hearing loss in blast injury and in chemotherapy treatment reveal strong covariation between hearing impairment and tinnitus awareness. Surgical correction of conductive hearing loss by middle ear surgery and sensorineural hearing loss by cochlear implantation reduces tinnitus loudness. Thus, changes in hearing thresholds, irrespective of sensorineural, conductive or mixed pattern, can modulate tinnitus loudness up or down. Tinnitus modulation related to hearing change typically stabilizes within one year.

Subject methods and systems find use for diagnosing, detecting and/or monitoring Tinnitus, with or without hearing loss in an individual. Subject methods and systems find use for diagnosing, detecting, monitoring, and/or treating Tinnitus or diseases associated with tinnitus, with or without hearing loss in an individual. Non-limiting examples of related conditions affiliated with Tinnitus include vestibular disorders, audiological problems, and behavioral health issues, such as, but not limited to: hearing loss, Meniere's Disease, hyperacusis, Misophonia, Phonophobia, Depression, Anxiety, and Temporomandibular Joint Disorder (TMD).

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-33 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

### Aspects A

Aspect 1. A method of detecting Tinnitus in a subject, the method comprising: a) acquiring functional magnetic resonance imaging (fMRI) functional connectivity data or magnetoencephalographic imaging (MEGI) functional connectivity data of at least one of the caudate nucleus, the caudate head, the caudate body, the frontal lobe, and the auditory cortex regions of the brain of the subject; b) assessing the fMRI functional connectivity data or the MEGI functional connectivity data in at the at least one region of the brain; c) determining if the fMRI functional connectivity data or the MEGI functional connectivity data are above or below a reference level associated with at least one or more pathology profiles of Tinnitus, wherein at least one pathology profile of Tinnitus comprises: i) modulated fMRI functional connectivity between the caudate nucleus and the rest of the brain as compared to the reference level; ii) modulated MEGI functional connectivity in the frontal lobe as compared to the reference level; or iii) modulated MEGI functional connectivity in the auditory cortex regions as compared to the reference level.

Aspect 2. The method of Aspect 1, wherein the modulated fMRI functional connectivity comprises increased fMRI functional connectivity between the caudate nucleus and the auditory cortex region of the brain.

Aspect 3. The method of Aspect 1, wherein the modulated fMRI functional connectivity comprises decreased fMRI functional connectivity between the caudate nucleus and the frontal lobe region of the brain.

Aspect 4. The method of Aspect 1, wherein the modulated MEGI functional connectivity comprises increased MEGI functional connectivity in the frontal cortex of the frontal lobe region of the brain.

Aspect 5. The method of Aspect 1, wherein the modulated MEGI functional connectivity comprises increased MEGI functional connectivity in the auditory cortex of the temporal lobe region of the brain.

Aspect 6. The method of Aspect 1, wherein the modulated MEGI functional connectivity comprises decreased MEGI functional connectivity in the auditory cortex of the temporal lobe region of the brain.

Aspect 7. The method of Aspect 1, wherein the modulated MEGI functional connectivity comprises decreased MEGI functional connectivity in the frontal cortex of the frontal lobe region of the brain.

Aspect 8. The method of any one of Aspects 1-7, wherein the at least one region of the brain is at least two regions of the brain.

Aspect 9. The method of any one of Aspects 1-8, wherein the method further comprises recording auditory-evoked field (AEF) peak latency in the subject in response to a pure-tone stimulus, wherein the AEF peaks are recorded using a MEGI imaging (MEGI) device.

Aspect 10. The method any one of Aspects 1-9, wherein the determining further comprises determining if the AEF peak latency in the subject is above or below a second reference level associated a second pathology profile of Tinnitus, wherein the second pathology profile comprises delayed latency of the AEF peaks in response to the pure-tone stimulus as compared to the second reference level.

Aspect 11. The method of any one of Aspects 1-10, wherein the fMRI functional connectivity data comprises oscillating neural signals between the auditory cortex and the rest of the brain.

Aspect 12. The method of any one of Aspects 1-11, wherein assessing the MEGI functional connectivity comprises assessing the hyposynchrony in the frontal cortex of the brain.

Aspect 13. The method of any one of Aspects 1-12, wherein assessing the hyposynchrony in the frontal cortex of the brain comprises assessing the global connectivity of the frontal cortex of the brain with the rest of the brain.

Aspect 14. The method of any one of Aspects 1-13, wherein the frontal cortex hyposynchrony magnitude is correlated with Tinnitus severity level.

Aspect 15. The method of any of the proceeding Aspects, wherein assessing the MEGI functional connectivity comprises assessing shifts in MEGI bandwidth frequencies in the frontal cortex as associated with the one or more pathology profiles of Tinnitus.

Aspect 16. The method of any one of Aspects 1-15, wherein decreased MEGI functional connectivity comprises decreased MEGI alpha-band activity ranging from 8-12 Hz.

Aspect 17. The method of any one of Aspects 1-16, wherein assessing the fMRI functional connectivity comprises assessing coherence between: a) the caudate nucleus and the auditory cortex; b) the caudate nucleus and the frontal lobe; or c) a combination thereof.

Aspect 18. The method of any one of Aspects 1-17, wherein assessing the fMRI functional connectivity comprises assessing hypoconnectivity between the caudate nucleus and the frontal lobe.

Aspect 19. The method of any one of Aspects 1-18, wherein assessing the fMRI functional connectivity comprises assessing hyperconnectivity between the caudate nucleus and the frontal lobe.

Aspect 20. The method any one of Aspects 1-19, wherein the one or more pathology profiles of Tinnitus is further associated with: a)modulated functional connectivity between the caudate nucleus and the cuneus region of the brain; b) modulated functional connectivity between the caudate nucleus and the superior lateral occipital cortex (sLOC); or c) modulated functional connectivity between the caudate nucleus and the anterior supramarginal gyrus (aSMG).

Aspect 21. The method of any of the proceeding Aspects, wherein the modulated functional connectivity comprises increased functional connectivity between the caudate nucleus and the cuneus region of the brain.

Aspect 22. The method of any of the proceeding Aspects, wherein modulated functional connectivity comprises increased functional connectivity between the caudate nucleus and the sLOC.

Aspect 23. The method of any of the proceeding Aspects, wherein modulated functional connectivity comprises increased functional connectivity between the caudate nucleus and the aSMG.

Aspect 24. The method of Aspect 9, wherein AEFs are evoked by the pure-tone stimulus at 1 kHz.

Aspect 25. The method of any one of Aspects 1-24, the method further comprises acquiring a plurality of high-resolution MR images.

Aspect 26. The method of Aspect 25, wherein the plurality of high-resolution MR images is reconstructed into three-dimensional images.

Aspect 27. The method of any one of Aspects 1-26, wherein the acquiring comprising acquiring the MEGI functional connectivity data with a resting-state MEGI imaging device (MEGI) with the subject's eyes closed.

Aspect 28. The method of any one of Aspects 24-27, wherein the recording comprises collecting the AEF peaks with the MEGI device with the subject's eyes open.

Aspect 29. The method of any of the proceeding Aspects, wherein the acquiring comprises acquiring the MEGI functional connectivity data with the subject's eyes closed.

Aspect 30. A method of analyzing images of the brain, the method comprising: (a) providing a database, using logistic regression algorithms, that comprises one or more pathology profiles associated with Tinnitus with or without hearing impairment; (b) receiving a plurality of functional magnetic resonance (fMR) images or functional magnetoencephalographic (MEG) images of at least one region of the brain; (c) analyzing the plurality of fMRI or MEGI images to obtain fMRI and MEGI functional connectivity data; and (d) comparing the fMRI or MEGI functional connectivity data from the fMRI or fMEGI images with the one or more pathology profiles of step (a).

Aspect 31. The method of Aspect 30, wherein the one or more pathology profiles is associated with acute or chronic tinnitus.

Aspect 32. The method of any one of Aspects 30-31, wherein the one or more pathology profiles is associated with hearing impairment.

Aspect 33. The method of Aspect 32, wherein hearing impairment comprises:i) acute or chronic hearing loss; ii) symmetric or asymmetric hearing loss; or iii) a combination thereof.

Aspect 34. The method of Aspect 33, wherein the one or more pathology profiles is associated with Tinnitus with or without hearing impairment.

Aspect 35. The method of any one of Aspects 30-34, wherein the one or more pathology profiles is derived from a plurality of fMRI or MEGI images of one or more subjects having the one or more pathology profiles.

Aspect 36. The method of Aspect 35, wherein the plurality of fMRI images are three dimensional images.

Aspect 37. The method of Aspect 35, wherein the plurality of MEGI images are three dimensional images.

Aspect 38. The method of any one of Aspects 30-37, further comprising receiving auditory-evoked field (AEF) data in response to a pure-tone stimulus.

Aspect 39. The method of Aspect 38, wherein the AEF data comprises AEF peaks corresponding to spatiotemporal auditory cortical activity.

Aspect 40. The method of any one of Aspects 38-39, wherein the database further comprises AEF data associated with the one or more pathology profiles.

Aspect 41. The method of any one of Aspects 38-40, wherein the method further comprises comparing latency of the AEF peaks in response to the pure-tone stimulus with the AEF data associated with the one or more pathology profiles.

Aspect 42. A method of analyzing fMRI signals or MEGI signals of the brain, the method comprising: (a) providing a database, using logistic regression algorithms, that comprises one or more pathology profiles associated with Tinnitus with or without hearing impairment; (b) receiving functional fMRI signals or functional MEGI signals from at least one region of the brain; (c) analyzing the plurality of fMRI or MEGI signals to obtain fMRI or MEGI functional connectivity data; and(d) comparing the fMRI or MEGI functional connectivity data from the fMRI or MEGI signals with the one or more pathology profiles of step (a).

Aspect 43. A multimodal automated system for determining the presence of Tinnitus in the subject, the system comprising: a) a functional magnetic resonance imaging (fMRI) device or a magnetoencephalographic imaging (MEGI) device; b) at least one memory storage medium configured to store functional connectivity data of the brain of the subject received from the fMRI or MEGI device; e) at least one processor operably coupled to the at least one memory storage medium, the at least one processor being configured to: i) process fMRI data or MEGI data recorded from at least one region of the brain in an individual, thereby generating fMRI functional connectivity data or MEGI functional connectivity data; ii) analyze fMRI or MEGI functional connectivity data, iii) prune, using logistic regression algorithms, the fMRI functional connectivity data or the MEGI functional connectivity data; iv) compare the fMRI or MEGI functional connectivity data obtained in step iii with functional connectivity data obtained from a database comprising one or more pathology profiles associated with Tinnitus; and v) determine if the individual has Tinnitus based on the data obtained in step iv.

Aspect 44. The system of Aspect 43, wherein the one or more pathology profiles is further associated with hearing impairment.

Aspect 45. The system of any one of Aspects 43-44, wherein the processor is further configured to identify latencies of the auditory-evoked field (AEF) peaks recorded from the auditory cortex of the individual in response to a pure-tone stimulus.

Aspect 46. The system of any one of Aspects 43-45, wherein the at least one region of the brain comprises the: a) caudate nucleus region of the brain; b) caudate head region of the brain; c) caudate body region of the brain; d) auditory cortex region of the brain; e) frontal lobe region of the brain; f) superior occipital cortex region of the brain; g) cuneus region of the brain; or h) a combination thereof.

Aspect 47. The system of any one of Aspects 43-46, wherein the MEGI functional connectivity data is recorded in the frontal cortex of the frontal lobe region of the brain.

Aspect 48. The system of any one of Aspects 43-46, wherein the MEGI functional connectivity data is recorded in the left and right superior frontal gyrus region of the frontal lobe.

Aspect 49. The system of any of the proceeding Aspects, wherein the processing fMRI data comprises linearly detrending and bandpass filtering the fMRI data or MEGI data.

Aspect 50. The system of any of the proceeding Aspects, wherein the fMRI functional connectivity data comprises a plurality of images.

Aspect 51. The system of any of the proceeding Aspects, wherein the MEGI functional connectivity data comprises a plurality of images.

Aspect 52. The system of any of the proceeding Aspects, wherein the processor is further configured to define seed regions within the plurality of images: i) anatomically based on subdivisions of the caudate nucleus of the rest of the brain; and ii) functionally using localizers for the auditory cortex auditory-evoked field (AEF) data recorded from the auditory cortex of the individual in response to a pure-tone stimulus.

Aspect 53. The system of any of the proceeding Aspects, wherein the processor is further configured to define seed regions using a statistical map and stereotactic coordinates of the at least one region of the brain.

Aspect 54. The system of any one of Aspects 43-53, wherein the comparing further comprises comparing the AEF latency peaks from the individual with one or more latency peaks AEF latency peaks obtained from the database.

Aspect 55. The system of any of the proceeding Aspects, wherein the logistic regression algorithm is a linear least squares regression, robust linear regression, support vector machine, k-means clustering, or ridge regression.

Aspect 56. The system of any one of Aspects 43-55, wherein the logistic regression algorithm comprises a plurality of logistic regression models.

Aspect 57. The system of any one of Aspects 43-56, wherein the logistic regression algorithm is a relevance vector machine that executes automatic feature pruning.

Aspect 58. The system of any one of Aspects 43-56, wherein the logistic regression algorithm deploys variants of relevance vector machines to perform pruning.

Aspect 59. The system of Aspect 56, wherein at least one or the plurality of logistic regression models comprises predictor variables of functional connectivity data.

Aspect 60. The system of any of the proceeding Aspects, wherein the functional connectivity at each oscillatory frequency is quantified by averaging an imaginary component of coherence across a plurality of seeds.

Aspect 61. A multimodal neuroimaging system, the system comprising: a) a functional magnetic resonance imaging (fMRI) device or a magnetoencephalographic imaging (MEGI) device; b) at least one memory storage medium configured to store functional connectivity data of the brain of the subject received from the fMRI or MEGI device; c) at least one processor operably coupled to the at least one memory storage medium, the at least one processor being configured to: i) process fMRI data or MEGI data recorded from at least one region of the brain in an individual, thereby generating fMRI functional connectivity data or MEGI functional connectivity data; ii) analyze fMRI or MEGI functional connectivity data, iii) prune, using logistic regression algorithms, the fMRI functional connectivity data or the MEGI functional connectivity data; iv) compare the fMRI or MEGI functional connectivity data obtained in step iii with functional connectivity data obtained from a database comprising one or more pathology profiles associated with Tinnitus; and v) determine if the individual has Tinnitus based on the data obtained in step iv.

Aspect 62. A neuroimaging system, the system comprising: a) a functional magnetic resonance imaging (fMRI) device; b) a processor; and c) a non-transient computer-readable medium comprising instructions that, when executed by the processor, cause the processor to: i) process fMRI functional connectivity data of a brain of an individual, thereby generating fMRI functional connectivity data for at least one region of the brain ii) analyze the fMRI functional connectivity data; and iii) determine if the individual has Tinnitus based on a binomial logistic regression model of functional connectivity between the caudate and auditory cortex region of the brain, wherein the binomial logistic regression model comprises functional connectivity values from bihemispheric caudate connectivity maps extracted from the ipsilateral posterior middle temporal gyrus of the brain.

Aspect 63. A non-transitory computer-readable memory medium comprising instructions that when executed cause a processor to: i) process fMRI data or MEGI data recorded from at least one region of the brain in an individual, thereby generating fMRI functional connectivity data or MEGI functional connectivity data; ii) analyze fMRI or MEGI functional connectivity data, iii) prune, using logistic regression algorithms, the FMRI functional connectivity data or the MEGI functional connectivity data; iv) compare the fMRI or MEGI functional connectivity data obtained in step iii with functional connectivity data obtained from a database comprising one or more pathology profiles associated with Tinnitus; and v) determine if the individual has Tinnitus based on the data obtained in step iv.

Aspect 64. A non-transitory computer-readable memory medium comprising instructions that when executed cause a processor to: i) process fMRI data recorded from at least one region of the brain in an individual, thereby generating fMRI functional connectivity data; ii) analyze fMRI functional connectivity data, iii) prune, using logistic regression algorithms, the fMRI functional connectivity data; iv) compare the fMRI functional connectivity data obtained in step iii with functional connectivity data obtained from a database comprising one or more pathology profiles associated with Tinnitus; and v) determine if the individual has Tinnitus based on the data obtained in step iv.

Aspect 65. A non-transitory computer-readable memory medium comprising instructions that when executed cause a processor to: i) process MEGI data recorded from at least one region of the brain in an individual, thereby generating MEGI functional connectivity data; ii) analyze MEGI functional connectivity data, iii) prune, using logistic regression algorithms, the MEGI functional connectivity data; iv) compare the MEGI functional connectivity data obtained in step iii with functional connectivity data obtained from a database comprising one or more pathology profiles associated with Tinnitus; and v) determine if the individual has Tinnitus based on the data obtained in iv.

Aspects B

Aspect 1. A method of detecting Tinnitus in a subject, the method comprising: a) acquiring functional magnetic resonance imaging (fMRI) functional connectivity data or magnetoencephalographic imaging (MEGI) functional connectivity data of at least one of the caudate nucleus, the caudate head, the caudate body, the frontal lobe, and the auditory cortex regions of the brain of the subject; b) assessing the fMRI functional connectivity data or the MEGI functional connectivity data in at the at least one region of the brain; c) determining if the fMRI functional connectivity data or the MEGI functional connectivity data are above, below, or at a reference level associated with at least one or more pathology profiles of Tinnitus, wherein at least one pathology profile of Tinnitus comprises: i) modulated fMRI functional connectivity between the caudate nucleus and the rest of the brain as compared to the reference level; ii) modulated MEGI functional connectivity in the frontal lobe as compared to the reference level; or iii) modulated MEGI functional connectivity in the auditory cortex regions as compared to the reference level.

Aspect 2. The method of Aspect 1, wherein the modulated fMRI functional connectivity comprises increased fMRI functional connectivity between the caudate nucleus and the auditory cortex region of the brain.

Aspect 3. The method of Aspect 1, wherein the modulated fMRI functional connectivity comprises decreased fMRI functional connectivity between the caudate nucleus and the frontal lobe region of the brain.

Aspect 4. The method of Aspect 1, wherein the modulated MEGI functional connectivity comprises increased MEGI functional connectivity in the frontal cortex of the frontal lobe region of the brain.

Aspect 5. The method of Aspect 1, wherein the modulated MEGI functional connectivity comprises increased MEGI functional connectivity in the auditory cortex of the temporal lobe region of the brain.

Aspect 6. The method of Aspect 1, wherein the modulated MEGI functional connectivity comprises decreased MEGI functional connectivity in the auditory cortex of the temporal lobe region of the brain.

Aspect 7. The method of Aspect 1, wherein the modulated MEGI functional connectivity comprises decreased MEGI functional connectivity in the frontal cortex of the frontal lobe region of the brain.

Aspect 8. The method of any one of Aspects 1-7, wherein the at least one region of the brain is at least two regions of the brain.

Aspect 9. The method of any one of Aspects 1-8, wherein the method further comprises recording auditory-evoked field (AEF) peak latency in the subject in response to a pure-tone stimulus, wherein the AEF peaks are recorded using a MEGI imaging (MEGI) device.

Aspect 10. The method any one of Aspects 1-9, wherein the determining further comprises determining if the AEF peak latency in the subject is above, below, or at a second reference level associated a second pathology profile of Tinnitus, wherein the second pathology profile comprises delayed latency of the AEF peaks in response to the pure-tone stimulus as compared to the second reference level.

Aspect 11. The method of any one of Aspects 1-10, wherein the fMRI functional connectivity data comprises oscillating neural signals between the auditory cortex and the rest of the brain.

Aspect 12. The method of any one of Aspects 1-11, wherein assessing the

MEGI functional connectivity comprises assessing the hyposynchrony in the frontal cortex of the brain.

Aspect 13. The method of any one of Aspects 1-12, wherein assessing the hyposynchrony in the frontal cortex of the brain comprises assessing the global connectivity of the frontal cortex of the brain with the rest of the brain.

Aspect 14. The method of any one of Aspects 1-13, wherein the frontal cortex hyposynchrony magnitude is correlated with Tinnitus severity level.

Aspect 15. The method of any of the proceeding Aspects, wherein assessing the MEGI functional connectivity comprises assessing shifts in MEGI bandwidth frequencies in the frontal cortex as associated with the one or more pathology profiles of Tinnitus.

Aspect 16. The method of any one of Aspects 1-15, wherein decreased MEGI functional connectivity comprises decreased MEGI alpha-band activity ranging from 8-12 Hz.

Aspect 17. The method of any one of Aspects 1-16, wherein assessing the fMRI functional connectivity comprises assessing coherence between: a) the caudate nucleus and the auditory cortex; b) the caudate nucleus and the frontal lobe; or c) a combination thereof.

Aspect 18. The method of any one of Aspects 1-17, wherein assessing the fMRI functional connectivity comprises assessing hypoconnectivity between the caudate nucleus and the frontal lobe.

Aspect 19. The method of any one of Aspects 1-18, wherein assessing the fMRI functional connectivity comprises assessing hyperconnectivity between the caudate nucleus and the frontal lobe.

Aspect 20. The method any one of Aspects 1-19, wherein the one or more pathology profiles of Tinnitus is further associated with: a) modulated functional connectivity between the caudate nucleus and the cuneus region of the brain; b) modulated functional connectivity between the caudate nucleus and the superior lateral occipital cortex (sLOC); or c) modulated functional connectivity between the caudate nucleus and the anterior supramarginal gyrus (aSMG).

Aspect 21. The method of any of the proceeding Aspects, wherein the modulated functional connectivity comprises increased functional connectivity between the caudate nucleus and the cuneus region of the brain.

Aspect 22. The method of any of the proceeding Aspects, wherein modulated functional connectivity comprises increased functional connectivity between the caudate nucleus and the sLOC.

Aspect 23. The method of any of the proceeding Aspects, wherein modulated functional connectivity comprises increased functional connectivity between the caudate nucleus and the aSMG.

Aspect 24. The method of Aspect 9, wherein AEFs are evoked by the pure-tone stimulus at 1 kHz.

Aspect 25. The method of any one of Aspects 1-24, the method further comprises acquiring a plurality of high-resolution MR images.

Aspect 26. The method of Aspect 25, wherein the plurality of high-resolution MR images is reconstructed into three-dimensional images.

Aspect 27. The method of any one of Aspects 1-26, wherein the acquiring comprising acquiring the MEGI functional connectivity data with a resting-state MEGI imaging device (MEGI) with the subject's eyes closed.

Aspect 28. The method of any one of Aspects 24-27, wherein the recording comprises collecting the AEF peaks with the MEGI device with the subject's eyes open.

Aspect 29. The method of any of the proceeding Aspects, wherein the acquiring comprises acquiring the MEGI functional connectivity data with the subject's eyes closed.

Aspect 30. A method of analyzing images of the brain, the method comprising: (a) providing a database, using logistic regression algorithms, that comprises one or more pathology profiles associated with Tinnitus with or without hearing impairment; (b) receiving a plurality of functional magnetic resonance (fMR) images or functional magnetoencephalographic (MEG) images of at least one region of the brain; (c) analyzing the plurality of fMRI or MEGI images to obtain fMRI and MEGI functional connectivity data; and (d) comparing the fMRI or MEGI functional connectivity data from the fMRI or fMEGI images with the one or more pathology profiles of step (a).

Aspect 31. The method of Aspect 30, wherein the one or more pathology profiles is associated with acute or chronic tinnitus.

Aspect 32. The method of any one of Aspects 30-31, wherein the one or more pathology profiles is associated with hearing impairment.

Aspect 33. The method of Aspect 32, wherein hearing impairment comprises: i) acute or chronic hearing loss; ii) symmetric or asymmetric hearing loss; or iii) a combination thereof.

Aspect 34. The method of Aspect 33, wherein the one or more pathology profiles is associated with Tinnitus with or without hearing impairment.

Aspect 35. The method of any one of Aspects 30-34, wherein the one or more pathology profiles is derived from a plurality of fMRI or MEGI images of one or more subjects having the one or more pathology profiles.

Aspect 36. The method of Aspect 35, wherein the plurality of fMRI images are three dimensional images.

Aspect 37. The method of Aspect 35, wherein the plurality of MEGI images are three dimensional images.

Aspect 38. The method of any one of Aspects 30-37, further comprising receiving auditory-evoked field (AEF) data in response to a pure-tone stimulus.

Aspect 39. The method of Aspect 38, wherein the AEF data comprises AEF peaks corresponding to spatiotemporal auditory cortical activity.

Aspect 40. The method of any one of Aspects 38-39, wherein the database further comprises AEF data associated with the one or more pathology profiles.

Aspect 41. The method of any one of Aspects 38-40, wherein the method further comprises comparing latency of the AEF peaks in response to the pure-tone stimulus with the AEF data associated with the one or more pathology profiles.

Aspect 42. A method of analyzing fMRI signals or MEGI signals of the brain, the method comprising: (a) providing a database, using logistic regression algorithms, that comprises one or more pathology profiles associated with Tinnitus with or without hearing impairment; (b) receiving functional fMRI signals or functional MEGI signals from at least one region of the brain; (c) analyzing the plurality of fMRI or MEGI signals to obtain fMRI or MEGI functional connectivity data; and (d) comparing the fMRI or MEGI functional connectivity data from the fMRI or MEGI signals with the one or more pathology profiles of step (a).

Aspect 43. A multimodal automated system for determining the presence of Tinnitus in the subject, the system comprising: a) a functional magnetic resonance imaging (fMRI) device or a magnetoencephalographic imaging (MEGI) device; b) at least one memory storage medium configured to store functional connectivity data of the brain of the subject received from the fMRI or MEGI device; c) at least one processor operably coupled to the at least one memory storage medium, the at least one processor being configured to: i) process fMRI data or MEGI data recorded from at least one region of the brain in an individual, thereby generating fMRI functional connectivity data or MEGI functional connectivity data; ii) analyze fMRI or MEGI functional connectivity data, iii) prune, using logistic regression algorithms, the fMRI functional connectivity data or the MEGI functional connectivity data; iv) compare the fMRI or MEGI functional connectivity data obtained in step iii with functional connectivity data obtained from a database comprising one or more pathology profiles associated with Tinnitus; and v) determine if the individual has Tinnitus based on the data obtained in step iv.

Aspect 44. The system of Aspect 43, wherein the one or more pathology profiles is further associated with hearing impairment.

Aspect 45. The system of any one of Aspects 43-44, wherein the processor is further configured to identify latencies of the auditory-evoked field (AEF) peaks recorded from the auditory cortex of the individual in response to a pure-tone stimulus.

Aspect 46. The system of any one of Aspects 43-45, wherein the at least one region of the brain comprises the: a) caudate nucleus region of the brain; b) caudate head region of the brain; c) caudate body region of the brain; d) auditory cortex region of the brain; e) frontal lobe region of the brain; f) superior occipital cortex region of the brain; g) cuneus region of the brain; or h) a combination thereof.

Aspect 47. The system of any one of Aspects 43-46, wherein the MEGI functional connectivity data is recorded in the frontal cortex of the frontal lobe region of the brain.

Aspect 48. The system of any one of Aspects 43-46, wherein the MEGI functional connectivity data is recorded in the left and right superior frontal gyrus region of the frontal lobe.

Aspect 49. The system of any of the proceeding Aspects, wherein the processing fMRI data comprises linearly detrending and bandpass filtering the fMRI data or MEGI data.

Aspect 50. The system of any of the proceeding Aspects, wherein the fMRI functional connectivity data comprises a plurality of images.

Aspect 51. The system of any of the proceeding Aspects, wherein the MEGI functional connectivity data comprises a plurality of images.

Aspect 52. The system of any of the proceeding Aspects, wherein the processor is further configured to define seed regions within the plurality of images: i) anatomically based on subdivisions of the caudate nucleus of the rest of the brain; and ii) functionally using localizers for the auditory cortex auditory-evoked field (AEF) data recorded from the auditory cortex of the individual in response to a pure-tone stimulus.

Aspect 53. The system of any of the proceeding Aspects, wherein the processor is further configured to define seed regions using a statistical map and stereotactic coordinates of the at least one region of the brain.

Aspect 54. The system of any one of Aspects 43-53, wherein the comparing further comprises comparing the AEF latency peaks from the individual with one or more latency peaks AEF latency peaks obtained from the database.

Aspect 55. The system of any of the proceeding Aspects, wherein the logistic regression algorithm is a linear least squares regression, robust linear regression, support vector machine, k-means clustering, or ridge regression.

Aspect 56. The system of any one of Aspects 43-55, wherein the logistic regression algorithm comprises a plurality of logistic regression models.

Aspect 57. The system of any one of Aspects 43-56, wherein the logistic regression algorithm is a relevance vector machine that executes automatic feature pruning.

Aspect 58. The system of any one of Aspects 43-56, wherein the logistic regression algorithm deploys variants of relevance vector machines to perform pruning.

Aspect 59. The system of Aspect 56, wherein at least one or the plurality of logistic regression models comprises predictor variables of functional connectivity data.

Aspect 60. The system of any of the proceeding Aspects, wherein the functional connectivity at each oscillatory frequency is quantified by averaging an imaginary component of coherence across a plurality of seeds.

Aspect 61. A multimodal neuroimaging system, the system comprising: a) a functional magnetic resonance imaging (fMRI) device or a magnetoencephalographic imaging (MEGI) device; b) at least one memory storage medium configured to store functional connectivity data of the brain of the subject received from the fMRI or MEGI device; c) at least one processor operably coupled to the at least one memory storage medium, the at least one processor being configured to: i) process fMRI data or MEGI data recorded from at least one region of the brain in an individual, thereby generating fMRI functional connectivity data or MEGI functional connectivity data; ii) analyze fMRI or MEGI functional connectivity data, iii) prune, using logistic regression algorithms, the fMRI functional connectivity data or the MEGI functional connectivity data; iv) compare the fMRI or MEGI functional connectivity data obtained in step iii with functional connectivity data obtained from a database comprising one or more pathology profiles associated with Tinnitus; and v) determine if the individual has Tinnitus based on the data obtained in step iv.

Aspect 62. A neuroimaging system, the system comprising: a) a functional magnetic resonance imaging (fMRI) device; b) a processor; and c) a non-transient computer-readable medium comprising instructions that, when executed by the processor, cause the processor to: i) process fMRI functional connectivity data of a brain of an individual, thereby generating fMRI functional connectivity data for at least one region of the brain ii) analyze the fMRI functional connectivity data; and iii) determine if the individual has Tinnitus based on a binomial logistic regression model of functional connectivity between the caudate and auditory cortex region of the brain, wherein the binomial logistic regression model comprises functional connectivity values from bihemispheric caudate connectivity maps extracted from the ipsilateral posterior middle temporal gyrus of the brain.

Aspect 63. A non-transitory computer-readable memory medium comprising instructions that when executed cause a processor to: i) process fMRI data or MEGI data recorded from at least one region of the brain in an individual, thereby generating fMRI functional connectivity data or MEGI functional connectivity data; ii) analyze fMRI or MEGI functional connectivity data, iii) prune, using logistic regression algorithms, the FMRI functional connectivity data or the MEGI functional connectivity data; iv) compare the fMRI or MEGI functional connectivity data obtained in step iii with functional connectivity data obtained from a database comprising one or more pathology profiles associated with Tinnitus; and v) determine if the individual has Tinnitus based on the data obtained in step iv.

Aspect 64. A non-transitory computer-readable memory medium comprising instructions that when executed cause a processor to: i) process fMRI data recorded from at least one region of the brain in an individual, thereby generating fMRI functional connectivity data; ii) analyze fMRI functional connectivity data, iii) prune, using logistic regression algorithms, the fMRI functional connectivity data; iv) compare the fMRI functional connectivity data obtained in step iii with functional connectivity data obtained from a database comprising one or more pathology profiles associated with Tinnitus; and v) determine if the individual has Tinnitus based on the data obtained in step iv.

Aspect 65. A non-transitory computer-readable memory medium comprising instructions that when executed cause a processor to: i) process MEGI data recorded from at least one region of the brain in an individual, thereby generating MEGI functional connectivity data; ii) analyze MEGI functional connectivity data, iii) prune, using logistic regression algorithms, the MEGI functional connectivity data; iv) compare the MEGI functional connectivity data obtained in step iii with functional connectivity data obtained from a database comprising one or more pathology profiles associated with Tinnitus; and v) determine if the individual has Tinnitus based on the data obtained in iv.

Aspect 66. A method of treating Tinnitus in a subject, the method comprising: a) acquiring functional magnetic resonance imaging (fMRI) functional connectivity data or magnetoencephalographic imaging (MEGI) functional connectivity data of at least one of the caudate nucleus, the caudate head, the caudate body, the frontal lobe, and the auditory cortex regions of the brain of the subject; b) assessing the fMRI functional connectivity data or the MEGI functional connectivity data in at the at least one region of the brain; c) determining if the fMRI functional connectivity data or the MEGI functional connectivity data are above, below, or at a reference level associated with at least one or more pathology profiles of Tinnitus, wherein at least one pathology profile of Tinnitus comprises: i) modulated fMRI functional connectivity between the caudate nucleus and the rest of the brain as compared to the reference level; ii) modulated MEGI functional connectivity in the frontal lobe as compared to the reference level; or iii) modulated MEGI functional connectivity in the auditory cortex regions as compared to the reference level; and d) delivering electrical, acoustic, or magnetic stimulation in one or more of the caudate nucleus, the caudate head, the caudate body, the frontal lobe, and the auditory cortex regions of the brain to reduce tinnitus loudness in the individual.

Aspect 67. The method of Aspect 66, wherein the electrical stimulation is deep brain stimulation (DBS).

Aspect 68. The method of Aspect 67, wherein the electrical stimulation is macrostimulation.

Aspect 69. The method of Aspect 66, wherein magnetic stimulation is generated by at least one of a Low Field Magnetic Stimulator (LFMS), a Magnetic Resonance Imager (MRI), a Transcranial Magnetic Stimulator (TMS), a Neuro-EEG synchronization Therapy device, or a combination thereof.

Aspect 70. The method of any one of Aspects 66-70, wherein said delivering stimulation comprises delivering one or more synchronized stimulations to the at least one or more of the caudate nucleus, the caudate head, the caudate body, the frontal lobe, and the auditory cortex regions of the brain.

Aspect 71. The method of any one of Aspects 66-70, wherein at least one synchronized stimulation comprises stimulation of multiple non-auditory pathways of 10 or more, 20 or more, or 30 or more locations across the caudate nucleus, the caudate head, the caudate body, the frontal lobe, and the auditory cortex regions of the brain.

Aspect 72. The method of any one of Aspects 66-68, wherein electrical stimulation is performed in one or more locations in the caudate body region of the brain.

Aspect 73. The method of any one of Aspects 66-68, wherein the electrical stimulation was performed in one or more locations in the caudate head or the brain.

The following examples are offered by way of illustration and not by way of limitation.

### EXAMPLES

### Example 1

### Striatal Gate Model

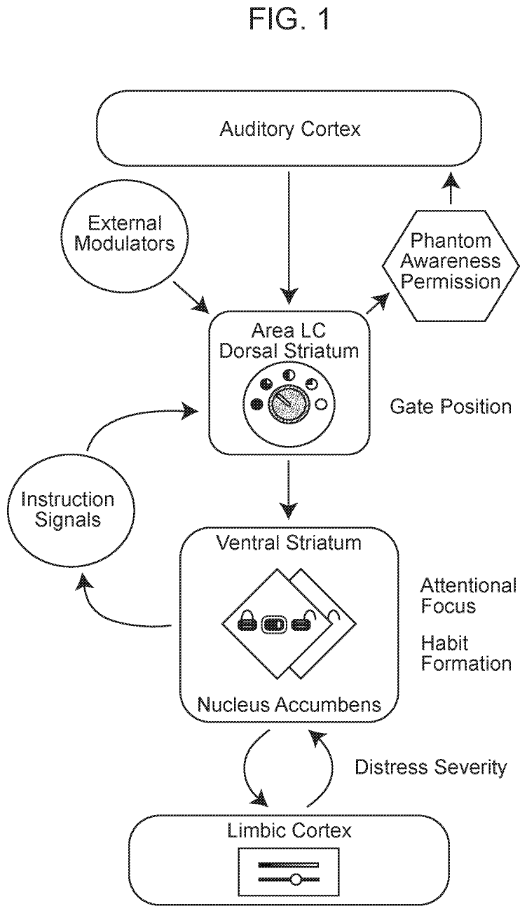

A diagnostic tool for detecting Tinnitus can be based on the following anchoring features of the striatal gate model (FIG. 1): instruction on details of phantom percepts are represented in the central auditory system, permission to gate candidate phantom percepts for conscious awareness is controlled by the dorsal striatum, action to attend, reject or accept phantom percepts, and form perceptual habits is decided by the ventral striatum, and determination of tinnitus distress severity is mediated through the limbic and paralimbic system-nucleus accumbens-ventral striatum loop.

Predictions arising from the striatal gate model are evaluable by multimodal neuroimaging and interventional neurostimulation methods. The latter include direct electrical (DBS), external magnetic (deep transcranial), external ultrasound (MRI guided ultrasound), and destructive lesion (GammaKnife) approaches. As such, the following anchoring features may be evaluated: 1) chronic tinnitus exhibits increased functional connectivity between striatum and auditory cortex; 2) dorsal striatal stimulation reduces tinnitus distress by altering strength of corticostriatal connectivity; 3) ventral striatal stimulation reduces tinnitus distress by altering attentional networks; and 4) cortical modulators external to the basal ganglia modify striatal gating function to effect tinnitus modulation.

### Example 2

### Caudate-Cortical Connectivity fMRI Differentiation Feature

At the cohort level, it has been demonstrated that caudate nucleus subdivision specificity of increased corticostriatal connectivity in chronic tinnitus. The striatal gate model was tested to examine the roles of auditory and auditory-limbic networks in chronic tinnitus noninvasively by comparing resting-state fMRI functional connectivity patterns in tinnitus patients against controls. Resting-state functional connectivity of the caudate dorsal striatum (area LC), caudate head (CH), nucleus accumbens (NA), and primary auditory cortex (A1) were tested to determine patterns of abnormal connectivity (Hinkley et al 2015 Front Hum Neurosci).

A comparison of chronic tinnitus patients adjusted for hearing loss levels with matched control subjects and normal hearing showed increased coherence between area LC and ipsilateral auditory cortical fields of the middle temporal gyrus (MTG) and superior temporal gyrus (STG). Increased coherence was specific to dorsal striatal area LC and was distinct from patterns of connectivity at other subdivisions of the basal ganglia, including the ventral striatum. Among other findings of increased connectivity between subdivisions of the basal ganglia and cortical areas, the area LC to auditory cortex network was unique, indicating its specificity to auditory phantoms. These findings provide growing support for a basal ganglia-centric model of chronic tinnitus. Abnormal caudate-cortical connectivity on resting-state fMRI was used as an anchoring feature to differentiate patients with tinnitus from those without tinnitus.

### Example 3

### fMRI and MEGI in Subjects with and without Tinnitus

fMRI cohort contrast studies that controlled for hearing loss level (moderate and unilateral profound hearing losses) to differentiate between tinnitus and no-tinnitus subjects showed nearly identical resting-state functional connectivity patterns. Intraoperative caudate nucleus stimulation experiments revealed caudate subdivision specificity of tinnitus modulation responses. fMRI study in moderately severe tinnitus subjects to contrast caudate head versus caudate body functional connectivity with auditory cortex confirmed the caudate body to be a more promising differentiation feature candidate. MEGI showed the left frontal gyrus to be correlated with tinnitus distress magnitude and increased latency of the peak M100 response to a 1 kHz tone differentiated chronic tinnitus subjects from controls. Those observations support the development of a multimodal neuroimaging-based objective tool to detect tinnitus that would be applicable across a wide range of hearing loss profiles.

### Example 4

### Increased Caudate to Auditory Cortex Connectivity in Tinnitus

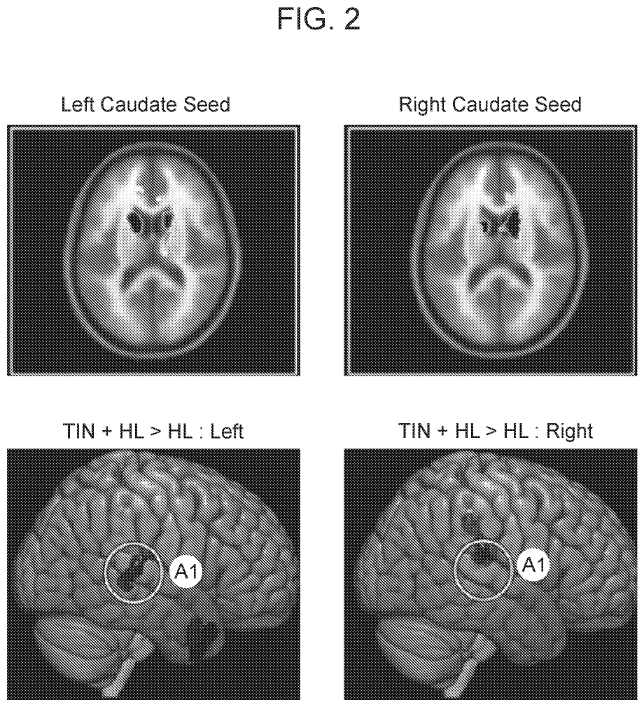

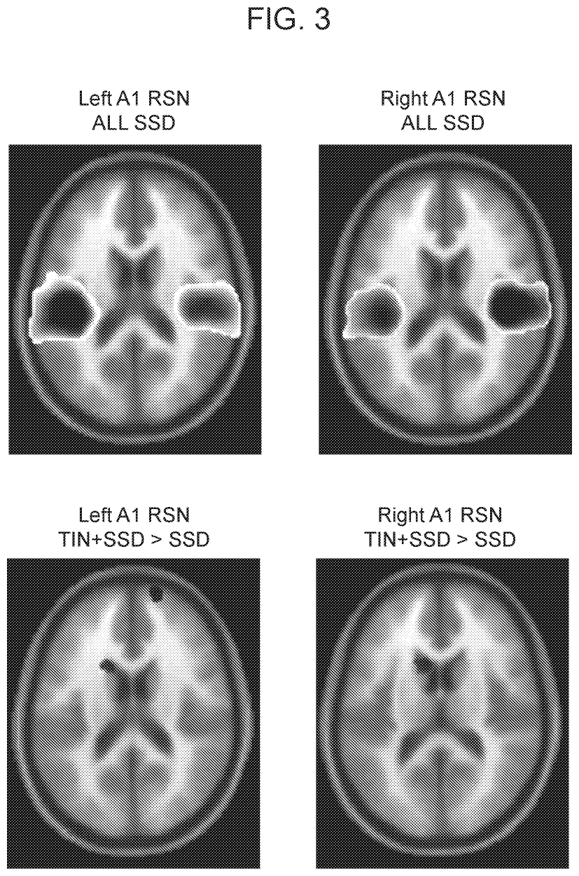

RS-fMRI increased connectivity between the caudate nucleus and auditory cortex differentiates between cohorts with tinnitus and hearing loss and hearing loss alone. In FIGS. 2 and 3, this robust feature is shown to remain valid across moderate hearing loss and unilateral hearing loss profiles. Abnormal corticostriatal functional connectivity serve as an anchoring feature in diagnostic tool construction.

FIG. 2 shows seeded regions of each caudate to have reciprocal patterns of connectivity with contralateral striatal structures in both cohorts, those with and without tinnitus. When subjects with tinnitus and moderate hearing loss (TIN+HL) and subjects with moderate hearing loss alone (HL) are compared, both the left and right caudate regions independently show increased (p<0.005) resting-state functional connectivity with primary auditory cortex (A1) in the chronic tinnitus cohort.

### Example 5

### Specificity of Increased Corticostriatal Connectivity in Tinnitus

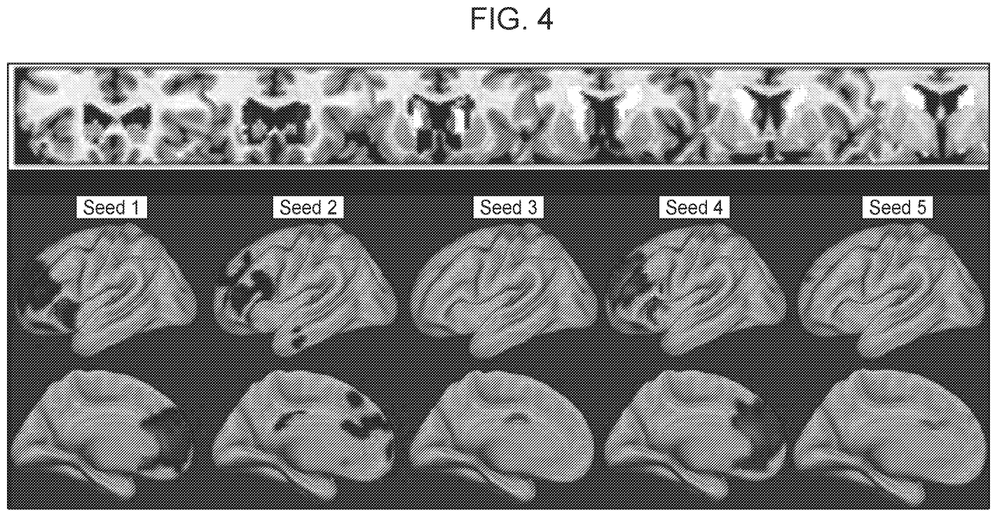

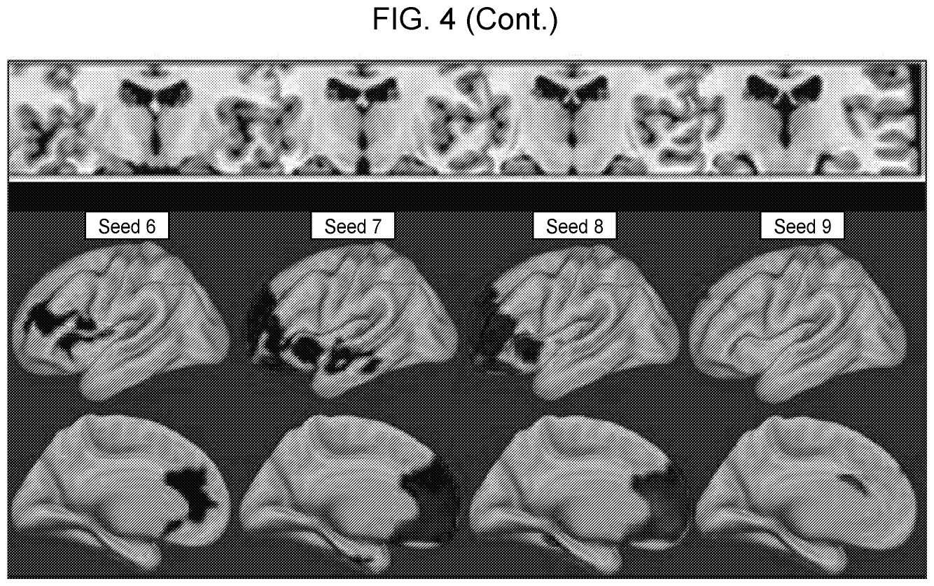

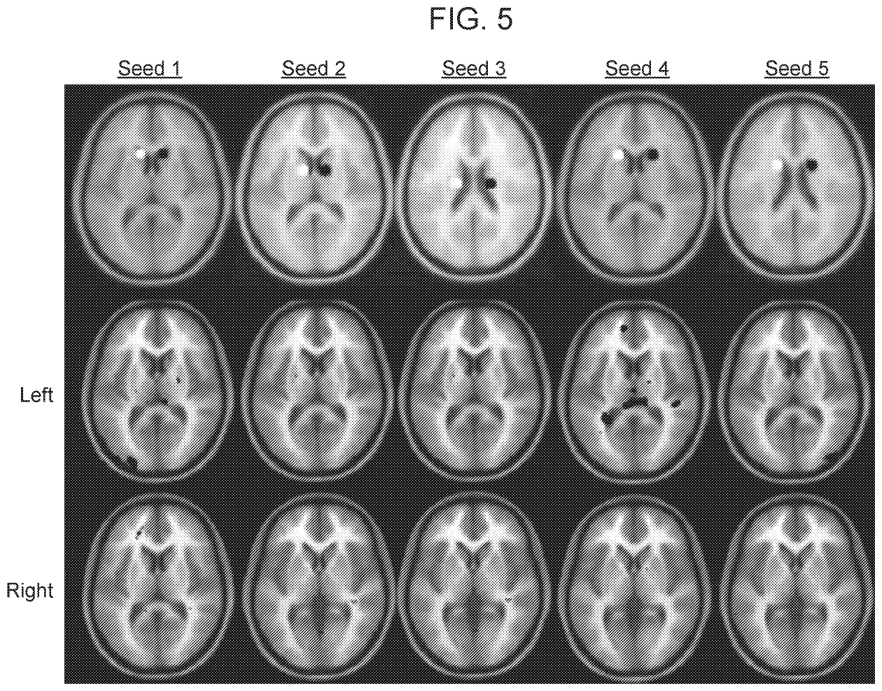

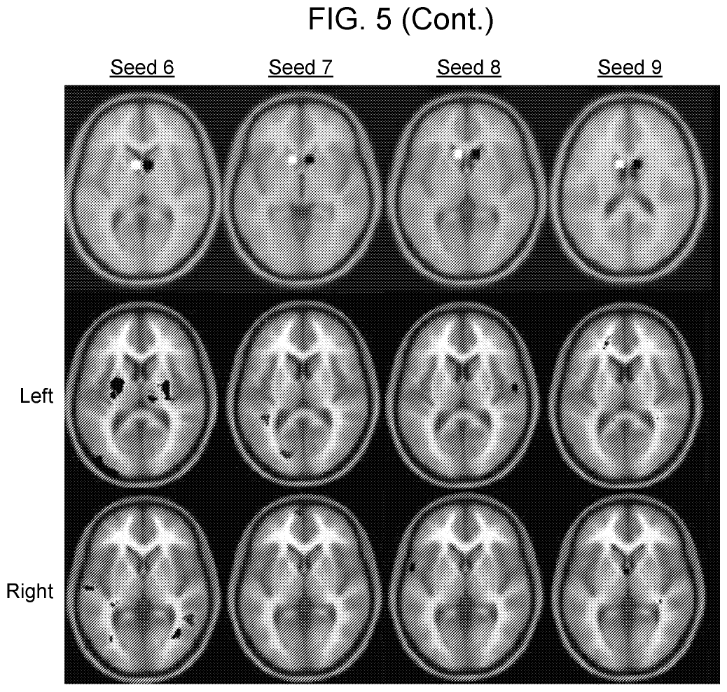

Specificity of fMRI increased corticostriatal connectivity within the caudate nucleus may be leveraged to improve diagnostic tool performance. In FIG. 4, upper row (Jung W H, Jang J H, Park J W, Kim E, Goo E H, Im O S, Kwon J S PLoS One. 2014; 9(9): e106768), the 9 subdivisions of the caudate nucleus functionally defined by FMRI are shown. In FIG. 4 middle and lower rows, a 5 mm radius sphere seed positioned at the centroid coordinate for each subdivision for cohorts with tinnitus and hearing loss (TIN+HL) and hearing loss alone (HL) confirm validity of caudate segmentation in chronic tinnitus. Distinct connectivity patterns are found for each separate seed. In FIG. 5, cohort contrast between TIN+HL and HL demonstrate specificity of increased corticostriatal connectivity to seed 6 and seed 7 in chronic tinnitus.

Subdivisions of the caudate nucleus exhibit distinct connectivity patterns that are common to cohorts with and without tinnitus. With this necessary background information established, direct contrasts between tinnitus and control cohorts may be made using either a whole brain approach or focused caudate subdivision seed-based approach. Those data-driven features will be added and pruned to augment tinnitus diagnostic tool construction.

### Example 6

### Caudate Segment Location and Tinnitus Modulation

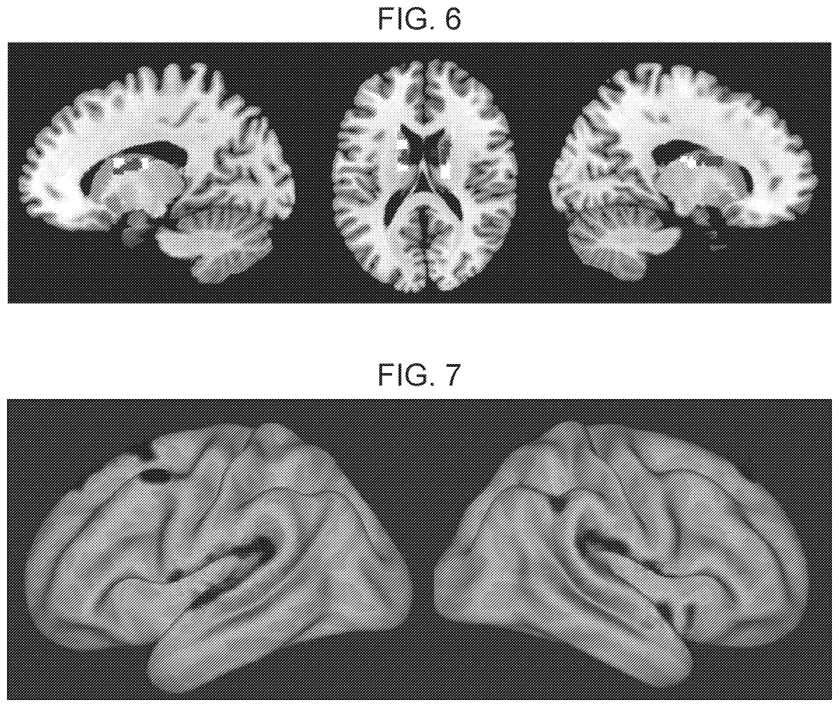

The relationship between caudate segment location and tinnitus modulation by direct stimulation provides important clinical context to the caudate subdivisions defined by fMRI. Six chronic tinnitus subjects who enrolled in an NIH-funded Phase I clinical trial of deep brain stimulation to treat moderately severe or worse medically refractory tinnitus underwent intraoperative stimulation of various locations along the anteroposterior axis of the caudate nucleus (Cheung et al, J Neurosurgery. 2019. In press). FIG. 6 plots the 20 locations that were systematically interrogated by positioning the DBS lead at the desired locale of the caudate nucleus and delivering broad stimulation under different frequency and intensity parameters. The primary acute stimulation outcome measure was reproducible tinnitus loudness reduction. Three of the four acute intraoperative responders (green) were positioned posteriorly, while all 16 non-responders (red) were positioned anteriorly in this limited sample. FIG. 7 shows a comparison of fMRI functional connectivity profiles of responders versus non-responders by seeding the centroids of respective clusters in 20 chronic tinnitus subjects with Tinnitus Functional Index scores>50, the minimum tinnitus severity level to enroll in the DBS Phase I trial. Acute tinnitus loudness reduction by direct basal ganglia stimulation is best realized in the caudate body subdivision, which has increased functional connectivity auditory cortex. Together, human physiological and neuroimaging functional connectivity evidence establishes the important relationship between increased corticostriatal functional connectivity and the likelihood of acute tinnitus loudness reduction with neuromodulation. As all tinnitus subjects in this study were without comorbid movement disorder conditions, these findings are likely applicable to most of the general population.

### Example 7

### Frontal Cortex Hyposynchrony and Tinnitus Severity

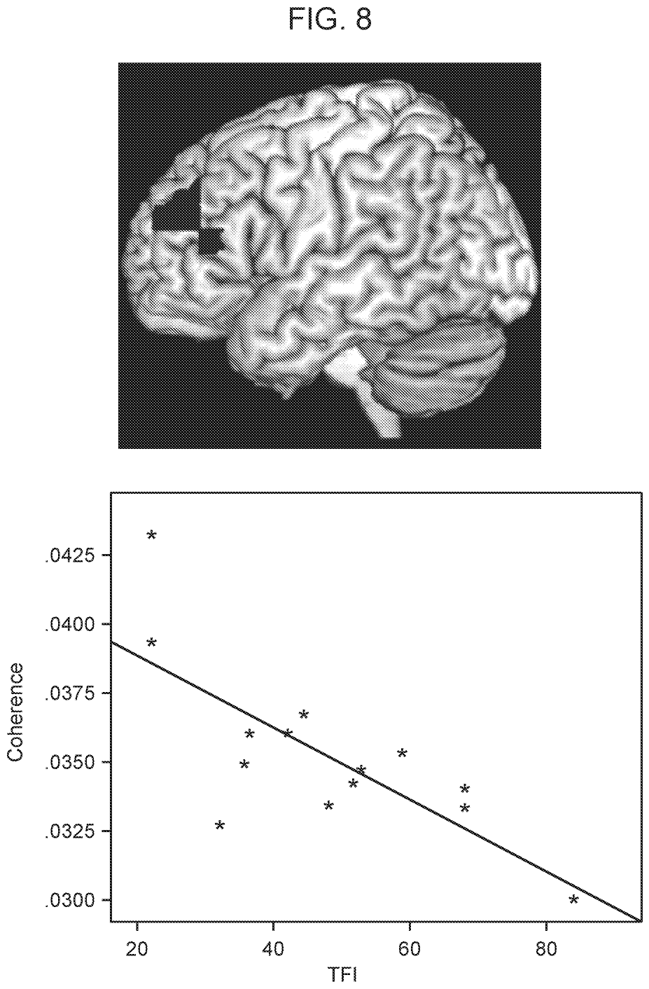

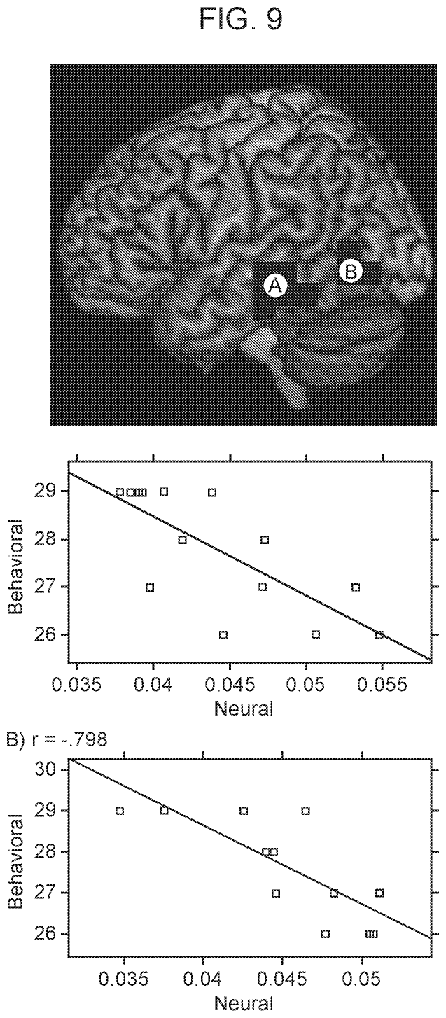

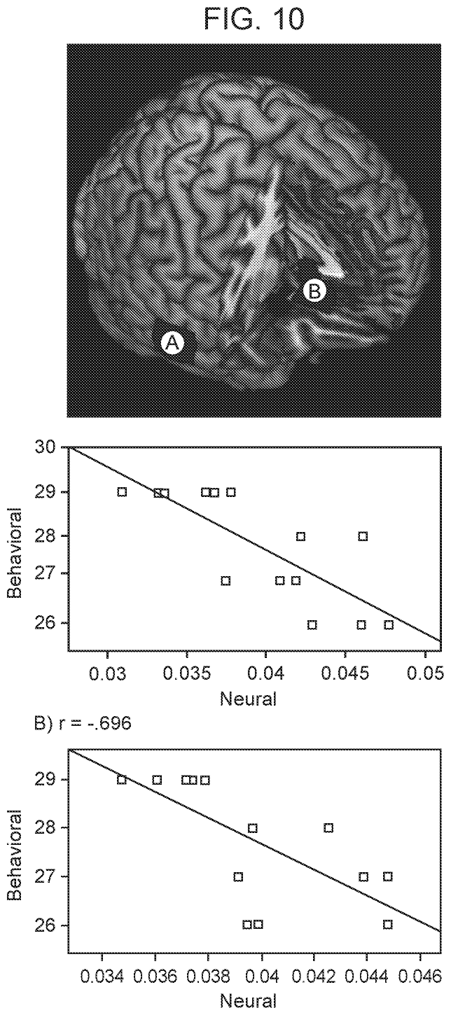

RS-MEGI left superior frontal gyrus functional connectivity strength is another candidate complementary anchoring feature of the tinnitus diagnostic tool. FIG. 8 shows this feature on whole brain MEGI, where frontal cortex hyposynchrony magnitude is correlated with tinnitus severity level.

### Example 8

### Delayed M100 Response to 1 kHz Tone in Tinnitus

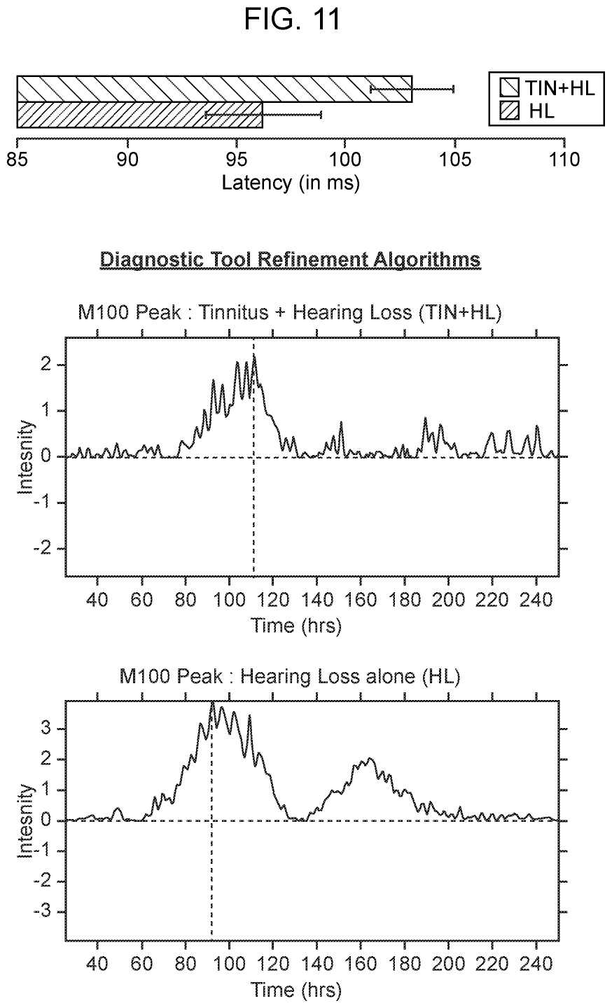

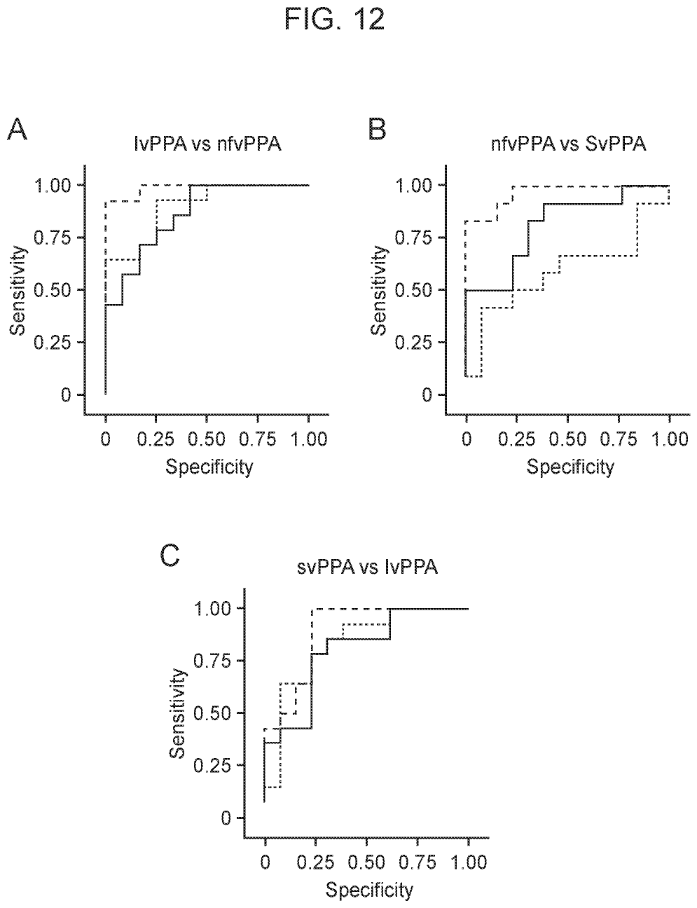

Spatiotemporal auditory cortical activity estimation from MEGI. Auditory evoked fields to evaluate the M100 response to a 1 kHz tone may serve as another anchoring feature of the tinnitus diagnostic tool. FIG. 11 shows increased M100 latency of the auditory evoked peak response in subjects with tinnitus and moderate hearing loss (TIN+HL) compared to subjects with moderate hearing loss alone (HL).

### Example 9

### Diagnostic Tool Refinement Algorithms

### Neuroimaging-Based Diagnostic Tool

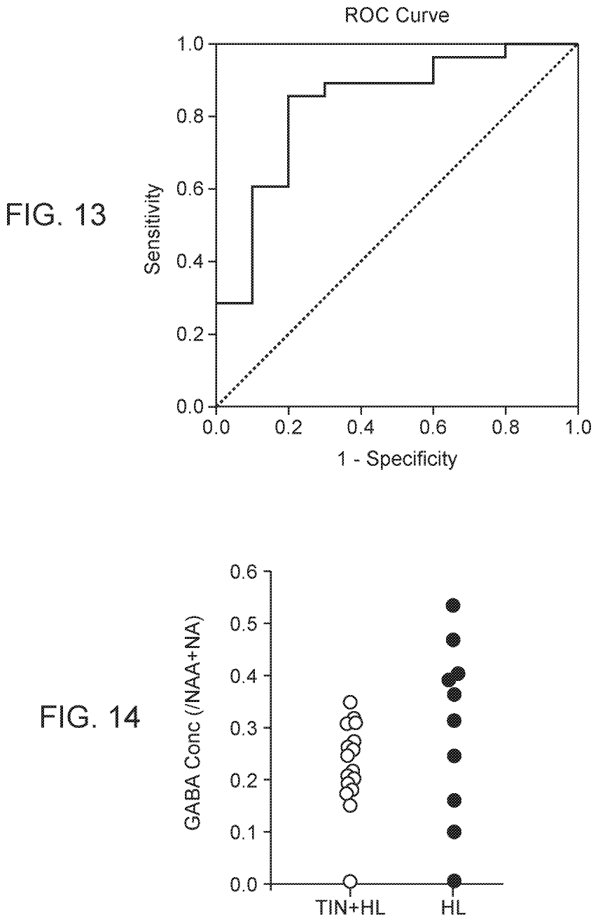

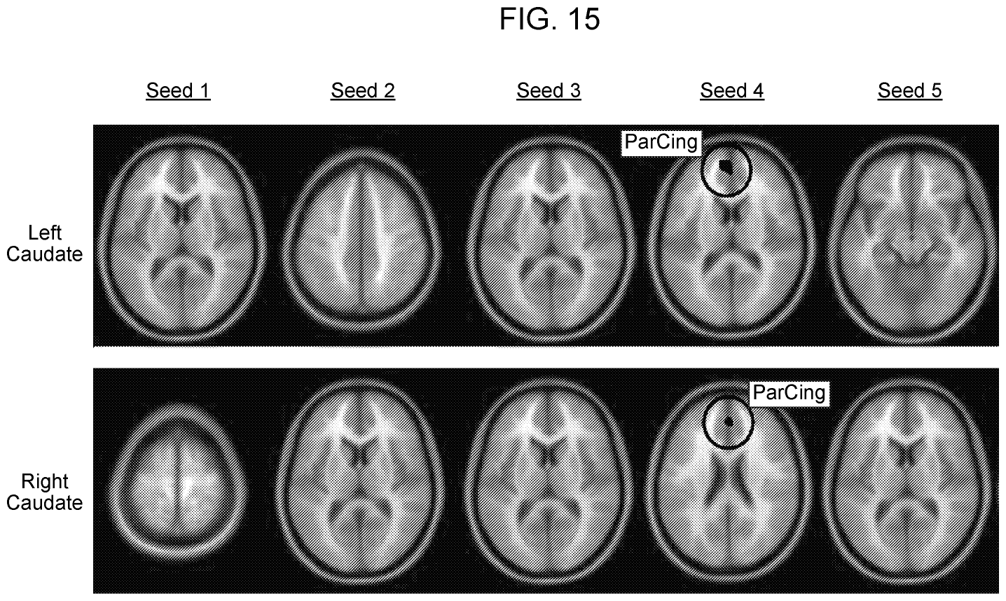

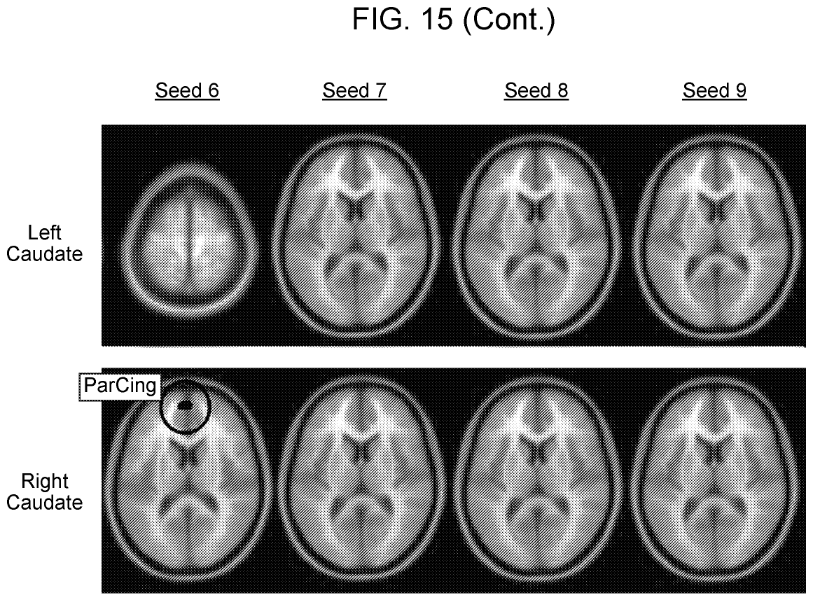

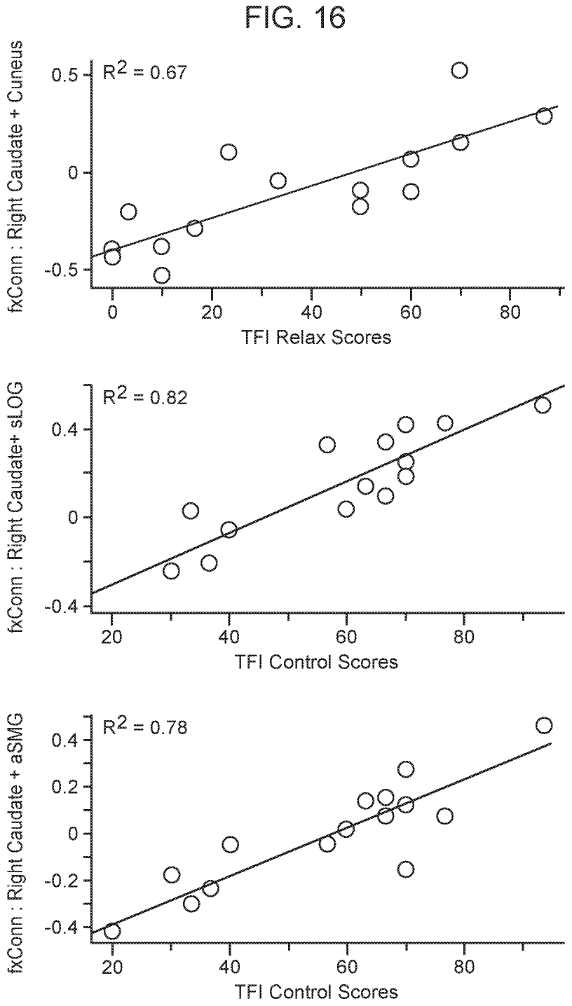

In subjects with tinnitus and without tinnitus that were controlled for moderate hearing loss, age, gender, and handedness, the tinnitus diagnostic tool performance of a binomial logistic regression model of functional connectivity between the caudate and auditory cortex was evaluated as an fMRI anchoring feature. Functional connectivity values (correlation coefficients) from the bihemispheric caudate connectivity maps were extracted from the ipsilateral posterior middle temporal gyrus and entered into our logistic regression model. The model was statistically significant (χ²=8.15, p<0.004) and performed very well with a sensitivity=96% and specificity=90%. FIG. 13 shows receiver operator characteristics, with an area under curve of 0.836 (p=0.002). These results provide strong evidence in favor of developing a robust tinnitus diagnostic tool anchored on neuroimaging data and augmented by behavioral and audiometric features. This objective tinnitus diagnostic tool is applicable to all adults, irrespective of hearing profile.

fMRI-Based Tinnitus Diagnostic Tool

In subjects with tinnitus and without tinnitus that were controlled for moderate hearing loss, age, gender, and handedness, the tinnitus diagnostic tool performance of a binomial logistic regression model of functional connectivity between the caudate and auditory cortex was evaluated as an fMRI anchoring feature. Functional connectivity values (correlation coefficients) from the bihemispheric caudate connectivity maps were extracted from the ipsilateral posterior middle temporal gyrus and entered into our logistic regression model. The model was statistically significant (χ²=8.15, p<0.004) and performed very well with a sensitivity=96% and specificity=90%. FIG. 13 shows receiver operator characteristics, with an area under curve of 0.836 (p=0.002). These preliminary results provide strong evidence in favor of the proposed approach for developing a robust tinnitus diagnostic tool anchored on neuroimaging data and augmented by behavioral and audiometric features. The objective was to construct a general tinnitus diagnostic tool applicable to all adults, irrespective of hearing profile.

### Example 10

### Imaging Procedures

Neuroimaging assessments of RS-MRI and RS-MEGI functional connectivity and spatiotemporal auditory cortical activity (MEG I) will be performed using established methods by the research team.

For RS-fMRI: Eight minutes (240 repetitions) of spontaneous 3T fMRI data (GE healthcare, Waukesha, Wis.) will be collected (supine position, eyes closed) with a gradient EPI sequence (2.0 mm×2.0 mm×2.0 mm slides, TR=2000 ms, TE=28 ms). Data from all voxels will be linearly detrended and bandpass filtered (2nd-order Butterworth; 0.01-0.08 Hz) prior to functional connectivity analysis. Seed regions will be defined both anatomically (e.g. subdivisions of the caudate, basal ganglia, and cortical regions) and functionally using localizers for auditory cortex obtained from task-based MEGI. Spatial extent and amplitude of resting-state connectivity networks, seeded from the basal ganglia and auditory cortex (Greicius et al., 2003 Proc Natl Acad Sci USA.; 100(1):253-8), will be evaluated using standard bivariate metrics such as correlation and coherence, as well as multivariate methods such as independent components analysis (Brookes et al. 2011, Neuroimage. 56(3):1082-104). Custom-built software tools are already in place for those tasks.

For Resting-state MEGI: Five minutes of resting-state MEGI data will be obtained in all subjects with their eyes closed and with their eyes open. Data will be collected at a sampling rate of 1 kHz. Source reconstruction algorithms will be used to reconstruct the electromagnetic neural activity at each brain voxel from the signal recorded by the entire MEGI sensor array (Dalal et al., 2008. Neuroimage; 40(4):1686-700; Dalal et al., 2011. Comput Intell Neurosci; 758973; Owen et al., 2012 Front Neurosci; 6: 186). A 3D grid of voxels with 2 mm spatial resolution covering the entire brain will be created for each subject and recording, based on a multisphere head model of coregistered structural 3D T1-weighted MR scans. Alignment of structural and functional images is ensured by marking 3 prominent anatomical points (nasion and both preauricular points) on the subject's head in MR images and localizing 3 fiducials attached to the same points before and after each MEGI scan Here, the focus was on functional connectivity of oscillating neural signals between auditory cortex and the rest of the brain (Guggisberg et al., 2008. Annals of Neurology; 63(2):193-203; Martino et al., 2011 Annals of Neurology; 69(3):521-532). An open-source toolbox, called NUTMEG, may be used for this analysis.

For spatiotemporal auditory cortical activity estimation from MEGI: Responses from bilateral auditory cortices in response to pure tone stimuli with frequencies will be chosen to be within their hearing range will also be sampled at 1 kHz with an MEGI sensor array of 275 axial magnetometers than span the whole scalp surface. Time-frequency dynamics of bihemispheric auditory cortices will be examined using standard methods (Dalal et al., 2008. Neuroimage; 40(4):1686-700; Owen et al., 2012 Front Neurosci; 6: 186). Auditory evoked fields task-based MEGI will localize primary and/or secondary auditory cortex bilaterally (Pross et al., 2015 Otol Neurotol 36(8):1443-1449; Chang et al., 2016 Laryngoscope 126(12):2785-2791) NUTMEG may be used for this analysis.

For High-resolution structural MRI: All MR studies will be performed using 32-channel receive-only array with a volume transmit head coil on a GE 3T scanner. For each subject, a high-resolution anatomical MRI will be acquired (MPRAGE; 160 1 mm slices, FOV=256 mm, TR=2300 ms, TE=2.98 ms). Additionally a gradient read-out echo sequence (GRE), a standard 2D, T2* weighted sequence, will be acquired in all subjects at 0.352×0.352 mm voxel size with a 512×512 matrix over an 18 cm field-of-view (FOV), ten 2 mm slices spaced 4 mm apart, an echo time (TE) of 11.4 ms, a repetition time (TR) of 250 ms, a 20° flip angle and 3 repetitions (number of excitations, NEX) in a 6.4 min scan. Custom-built software tools for this task have already been developed.

The sparse Bayesian classification algorithm demonstrates superior performance under a variety of simulation conditions. Performance evaluation criteria were error rates as a function of feature redundancy, sparsity, and signal dimension size. These algorithms may be used to prune large sets of hypothesis-driven, data-driven, psychometric and audiometric features to improve tinnitus diagnostic tool performance.

### Example 11

### Human Caudate Nucleus Subdivisions in Tinnitus Modulation

The purpose of this study was to define caudate nucleus locations responsive to intraoperative direct electrical stimulation for tinnitus loudness modulation and relate those locations to functional connectivity maps between caudate nucleus subdivisions and auditory cortex.

### Methods

Six awake study participants who underwent bilateral deep brain stimulation (DBS) electrode placement in the caudate nucleus as part of a phase I clinical trial were analyzed for tinnitus modulation in response to acute stimulation at 20 locations. Resting-state 3-T functional MRI (fMRI) was used to compare connectivity strength between centroids of tinnitus loudness-reducing or loudness-nonreducing caudate locations and the auditory cortex in the 6 DBS phase I trial participants and 14 other neuroimaging participants with a Tinnitus Functional Index>50.

### Study Participants

One hundred ninety-five prospective study were prescreened for this phase I clinical trial, which is registered with the ClinicalTrials.gov database (www(dot)clinicaltrials(dot)gov) and has a registration no. of NCT01988688. A large number of patients were eliminated from further consideration because of factors that included anxiety, depression, and expressed suicidality, yielding 14 prospective participants who advanced to comprehensive audiological and neuropsychological screening and resting-state 3-T fMRI. Nine study participants met eligibility criteria and 6 elected to proceed with DBS implantation between August 2014 and February 2017, providing tinnitus perceptual data in response to acute DBS electrode macrostimulation during surgery. Inclusion criteria included men and women between the ages of 22 and 75 years, subjective unilateral or bilateral nonpulsatile tinnitus of 1 year's duration or more, Tinnitus Functional Index (TFI)>50 (moderate problem or more severe), tinnitus unsatisfactorily responsive to acoustical or behavioral therapy, and Montreal Cognitive Assessment score≥26. Exclusion criteria included hyperacusis and profound hearing loss in both ears.

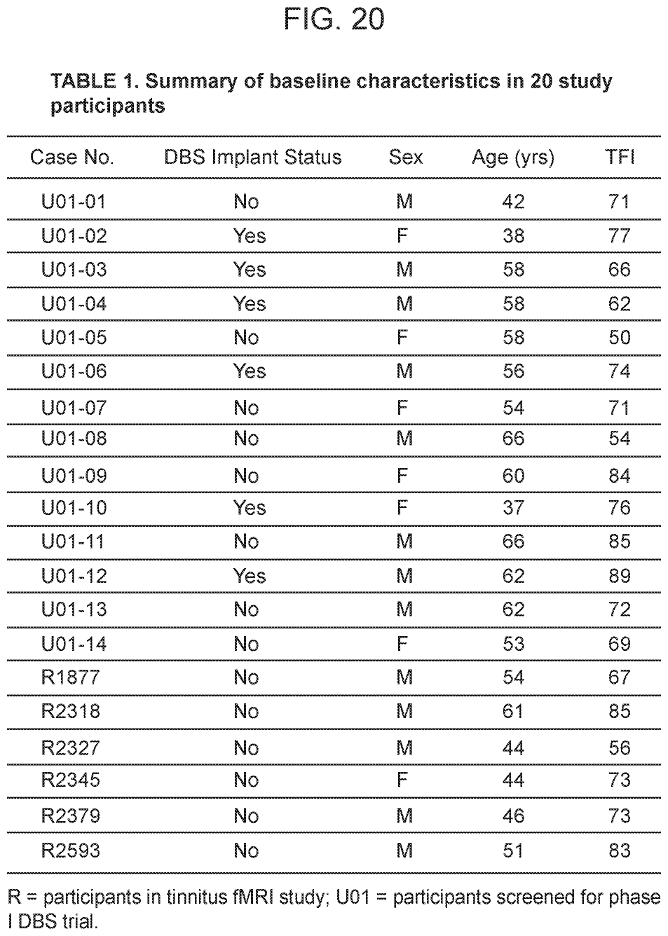

Two sets of experiments are reported herein. For the intraoperative data set to evaluate acute tinnitus loudness differences between the head and body subdivisions of the caudate nucleus in response to acute electrical stimulation, there were 6 participants (2 females) with a mean age (mean±standard deviation) of 51.5±11 years (range 37-62 years) and mean TFI of 74.2±9.8 (range 62-89). For the resting-state 3-T fMRI data set to evaluate auditory corticostriatal connectivity differences between caudate nucleus subdivisions, all 14 prospective participants were included, and another 6 tinnitus patients with TFI>50 who had participated in a neuroimaging study using the same scanner were added to the cohort, for a total of 20 patients (7 females) with a mean age of 53.5±8.9 years (range 37-66 years) and mean TFI of 71.9±10.8 (range 50-89; big problem or relatively severe). Baseline information for these 20 participants is summarized in Table 1 of FIG. 20. All participants gave written informed consent following explanation of the study procedures, which were approved by the UCSF Committee on Human Research. All experiments were conducted in accordance with the Declaration of Helsinki.

Caudate Nucleus Mapping with Electrical Stimulation

Awake stereotactic functional neurosurgery was performed using a Leksell frame (Elekta) and Framelink stereotactic software (Medtronic StealthStation). The caudate nucleus was targeted using an entry point at or just anterior to the coronal suture. A trajectory was planned to the subthalamic region, avoiding sulci, visible blood vessels, and the ventricles. The trajectory was then shortened to the caudate nucleus and medialized in the coronal plane to place the bottom of the trajectory at the base of the caudate. The depth of the trajectory was adjusted to center the 10.5-mm-long electrode array of a model 3387 DBS electrode (Medtronic) within the caudate nucleus in the coronal plane. Targeting was modified in the anteroposterior direction in the caudate nucleus to interrogate different loci as intraoperative mapping progressed. Microelectrode recording (MER) was performed using an Alpha Omega recording system (Alpha Omega Co.). A single MER pass was performed at the originally planned target in all cases. This was followed by placement of the DBS lead along the same tract, with the contacts spanning the caudate top to bottom in the coronal oblique trajectory plane based on the depth of the superior and inferior borders determined by MER. If stimulation-induced tinnitus loudness modulation (defined below) was observed at the original target, no further MER passes were made. If no significant tinnitus loudness modulation was observed, the DBS lead was removed and a second MER pass was performed along a parallel tract 5 mm anterior or posterior to the original target within the caudate. The DBS lead was placed in the second tract, and macrostimulation was again performed. This process was repeated until a location in the caudate that produced tinnitus modulation via macrostimulation was identified or a maximum of three passes were made per hemisphere. Bipolar macrostimulation was initially performed with the most distal contact (contact 0) set as the cathode and the most proximal contact (contact 3) set as the anode.

On a tinnitus loudness numeric rating scale (NRS) that ranged from 0 to 10 (0=no tinnitus, 5=conversation level, 10=jet engine), participants provided baseline values for both ears. Intraoperative use of the TFI, a 25-item validated instrument, was not feasible to assess tinnitus. The stimulation parameters of frequency, amplitude, and pulse width were varied only one at a time in a stepwise fashion, and study participants were queried to assess for any change in the tinnitus loudness rating. A total 2-point change from baseline summed across both ears was used as the threshold to determine stimulation-induced tinnitus loudness modulation. The receiver operating characteristics of this change in tinnitus loudness to change in tinnitus severity were as follows: sensitivity=0.84 and specificity=0.38.3 Tinnitus loudness modulation effects typically lasted no more than 1 minute following return of the stimulation amplitude to the lowest setting 2 V), which was performed without subject knowledge. The DBS electrode locations within the caudate nuclei were transformed from anatomical coordinates to normalized Montreal Neurological Institute (MNI) brain template coordinates for subsequent analysis.

Resting-State fMRI Data Acquisition

Data were collected using a GE Discovery 3-T MRI scanner (General Electric Healthcare MRI). Participants underwent both high-resolution fast spoiled gradient (FSPGR) brain volume (BRAVO) anatomical T1-weighted (0.5×⊐0.5 mm, TR=7 msec, TE=3 msec) and resting-state echo-planar imaging (EPI; 1.88×1.88 mm, 3-mm slice thickness, TR=2000 msec, TE=28 msec, 100 repetitions) sequences. Data were preprocessed using SPM12 (www(dot)fil(dot)ion(dot)ucl(dot)ac(dot)uk/spm/software/spm12/), and functional connectivity metrics were estimated using the CONN toolbox (www(dot)nitrc(dot)org/projects/conn).

### Data Preprocessing

Resting-state fMRI data were spatially preprocessed, and EPI images were spatially realigned to a mean image and co-registered with the T1-weighted image for each individual by using SPM8 (www(dot)fil(dot)ion(dot)ucl(dot)ac(dot)uk/spm/software/spm8/). Preprocessing with the default pipeline in the CONN functional connectivity toolbox included functional realignment and unwarp, slice-timing correction, structural segmentation and normalization, functional normalization, artifact detection tools (ART)-based functional outlier detection and scrubbing, and functional smoothing with an 8-mm Gaussian kernel in MNI space.

### Seed Definition

Seed regions were generated using the MarsBar Matlab toolbox (http://marsbar.sourceforge.net). A 5-mm-radius sphere was centered on a region of interest (ROI) defined by the average x, y, z coordinates of 1) the two left posterior DBS electrode locations that resulted in decreased tinnitus loudness, 2) the two right DBS electrode locations that resulted in decreased tinnitus loudness, 3) the nine left DBS electrode locations that did not result in decreased tinnitus loudness, and 4) the six right DBS electrode locations that did not result in decreased tinnitus loudness, for a total of four seed ROIs. The one left anterior DBS electrode location that resulted in decreased tinnitus loudness was treated as an outlier and was not included in the generation of seed regions.

### Functional Connectivity Analysis

The CONN toolbox was used for functional connectivity analysis. Seed-to-voxel analysis was performed to compare the positive contrast of functional networks connected to the more posterior caudate seed generated from DBS locations that had resulted in decreased tinnitus loudness and the more anterior caudate seed generated from DBS locations that had not resulted in decreased tinnitus loudness. Analyses were performed separately for the right and left hemispheres. Thresholds for differences were set at p<0.05 with an additional cluster correction threshold set at p<0.05 using a false discovery rate correction.

Tinnitus Loudness Modulation with Caudate Stimulation

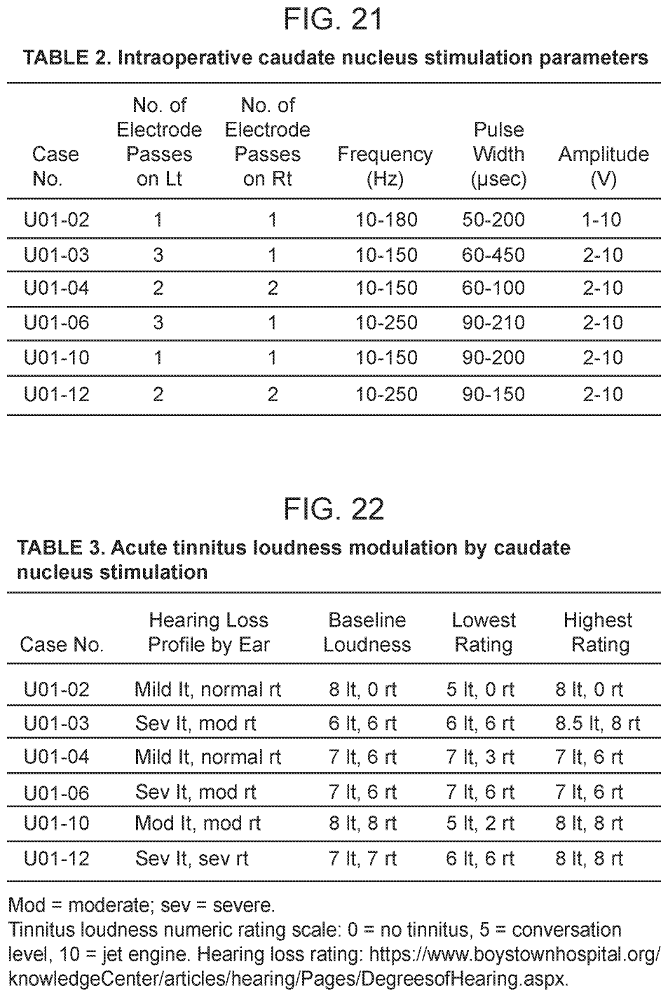

In the 6 participants who underwent DBS device implantation, acute tinnitus modulation by DBS electrode macrostimulation was assessed in 12 locations of the left caudate and 8 locations of the right caudate nuclei. All intraoperatively induced changes to tinnitus loudness perception returned to baseline values within 1 minute of returning the stimulation amplitude to the lowest level. The number of DBS electrode passes in the left and right hemispheres ranged between 1 and 3, and the stimulation parameters of frequency, pulse width, and amplitude varied widely (FIG. 21, Table 2). The hearing loss profile was asymmetrical in 4 participants (U01-02, -03, -04, and -06) with tinnitus loudness rated higher in the poorer ear in 3 of the 4 and was symmetrical in 2 participants (U01-10, -12) with tinnitus loudness rated at the same level in both ears (FIG. 22, Table 3). Reports of tinnitus loudness modulation, defined as a total 2-point change from baseline summed across both ears, or a change in tinnitus sound quality from awake participants during caudate nucleus mapping procedures guided final DBS electrode placement for long-term, chronic stimulation. During acute macrostimulation, 4 participants (U01-02, -04, -10, and -12) reported decreased tinnitus loudness at specific stimulation parameters (FIG. 22, Table 3). One of these 4 participants (U01-12) also reported increased tinnitus loudness. Among the remaining 2 participants, one (U01-06) reported no change in tinnitus loudness and one (U01-03) reported only increased tinnitus loudness.

### Caudate Subdivisions and Tinnitus Loudness Modulation

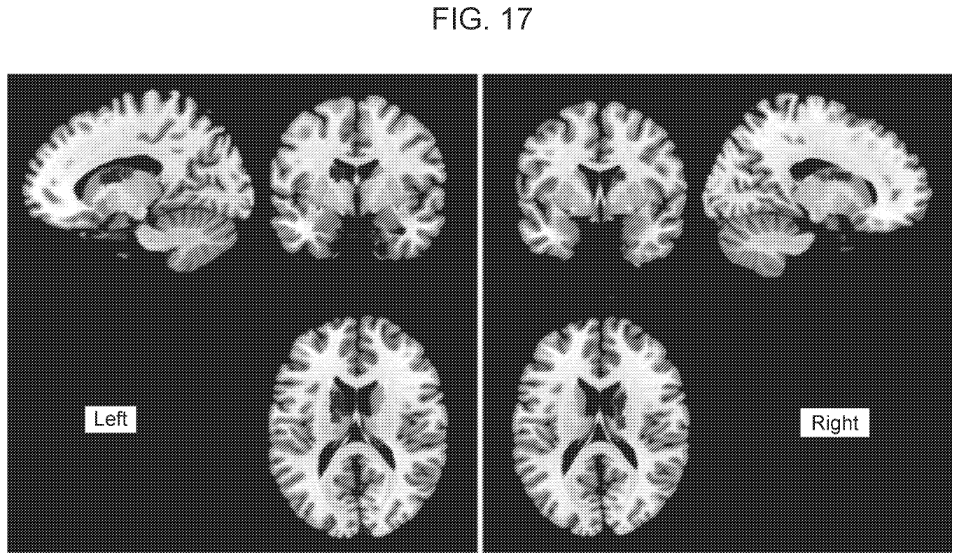

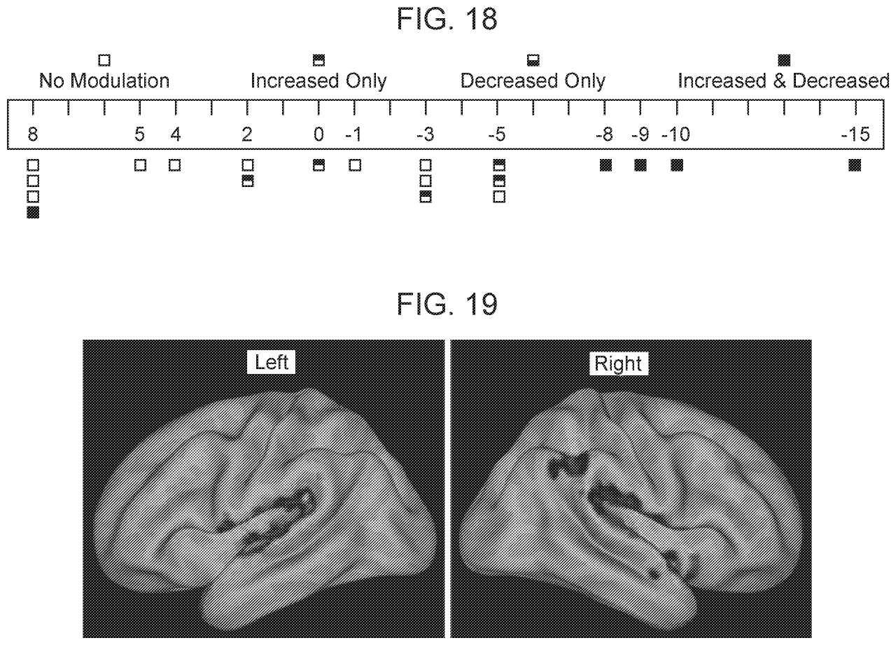

Macrostimulation at 5 DBS electrode locations resulted in decreased tinnitus loudness. The remaining 15 electrode locations resulted in either no change or increased tinnitus loudness. Four of the 5 electrode locations with decreased tinnitus loudness were positioned more posteriorly in the caudate body, whereas all 15 locations without decreased tinnitus loudness were located anteriorly, toward the caudate head. Electrode positions in the left and right caudate nuclei in MNI space with color coding of the stimulation locations with and without tinnitus loudness reduction are displayed in FIG. 17. By collapsing right and left hemispheric caudate nucleus interrogation data, a anteroposterior map of the caudate nucleus can be constructed for tinnitus modulation. The caudate nucleus head is anterior (positive) and the body is posterior (negative). Combined decreases and increases in tinnitus loudness modulation are strongly clustered for MNI coordinates in the caudate body subdivision, between −8 and −15 mm (FIG. 18).

Caudate Subdivisions and Resting-State fMRI Connectivity

Short-term tinnitus loudness reduction derived from acute intraoperative stimulation experiments motivated a comparison of functional connectivity patterns between the caudate head and body subdivisions. For this analysis, a total of 20 participants with chronic bothersome tinnitus defined as TFI>50 were evaluated using the CONN toolbox. The seeded connectivity analysis was performed for both hemispheres. While the right hemisphere did not show a statistically significant difference, the left hemisphere revealed the more posteriorly positioned caudate body to have increased auditory corticostriatal functional connectivity to the left and right superior temporal gyri as shown in FIG. 19.

By identifying a subdivision of the caudate nucleus in which acute electrical stimulation led to decreased tinnitus loudness and employing fMRI techniques to show that the caudate body has increased functional connectivity to auditory cortex, results that relate tinnitus perceptual data to functional neuroanatomy were demonstrated. This first-in-human striatal mapping study provides further insight into the corticostriatal networks involved in chronic bothersome tinnitus and enables more precise targeting for clinical intervention.

In this study, it was found that the posterior region of the caudate, corresponding to the body subdivision, modulated tinnitus loudness more consistently in response to electrical stimulation.

Acute DBS of the caudate nucleus in a small phase I clinical trial cohort reveals auditory phantom neuromodulatory and functional connectivity distinctions between the head and body subdivisions. The posteriorly located caudate body more reliably results in short-term tinnitus loudness reduction. Compared to the caudate head, the caudate body has stronger functional connectivity to the auditory cortex.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, Aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of detecting Tinnitus in a subject, the method comprising:
a) acquiring functional magnetic resonance imaging (fMRI) functional connectivity data or magnetoencephalographic imaging (MEGI) functional connectivity data of at least one of the caudate nucleus, the caudate head, the caudate body, the frontal lobe, and the auditory cortex regions of the brain of the subject;
b) assessing the fMRI functional connectivity data or the MEGI functional connectivity data in at the at least one region of the brain;
c) determining if the fMRI functional connectivity data or the MEGI functional connectivity data are above, below, or at a reference level associated with at least one or more pathology profiles of Tinnitus, wherein at least one pathology profile of Tinnitus comprises:
i) modulated fMRI functional connectivity between the caudate nucleus and the rest of the brain as compared to the reference level;
ii) modulated MEGI functional connectivity in the frontal lobe as compared to the reference level; or
iii) modulated MEGI functional connectivity in the auditory cortex regions as compared to the reference level.

2. The method of claim 1, wherein the modulated fMRI functional connectivity comprises increased fMRI functional connectivity between the caudate nucleus and the auditory cortex region of the brain.

3. The method of claim 1, wherein the modulated fMRI functional connectivity comprises decreased fMRI functional connectivity between the caudate nucleus and the frontal lobe region of the brain.

4. The method of claim 1, wherein the modulated MEGI functional connectivity comprises increased MEGI functional connectivity in the frontal cortex of the frontal lobe region of the brain.

5. The method of claim 1, wherein the modulated MEGI functional connectivity comprises increased MEGI functional connectivity in the auditory cortex of the temporal lobe region of the brain.

6. The method of claim 1, wherein the modulated MEGI functional connectivity comprises decreased MEGI functional connectivity in the auditory cortex of the temporal lobe region of the brain.

7. The method of claim 1, wherein the modulated MEGI functional connectivity comprises decreased MEGI functional connectivity in the frontal cortex of the frontal lobe region of the brain.

8. The method of claim 1, wherein the at least one region of the brain is at least two regions of the brain.

9. The method of claim 1, wherein the method further comprises recording auditory-evoked field (AEF) peak latency in the subject in response to a pure-tone stimulus, wherein the AEF peaks are recorded using a MEGI imaging (MEGI) device.

10. The method of claim 1, wherein the determining further comprises determining if the AEF peak latency in the subject is above, below, or at a second reference level associated a second pathology profile of Tinnitus, wherein the second pathology profile comprises delayed latency of the AEF peaks in response to the pure-tone stimulus as compared to the second reference level.

11. The method of claim 1, wherein the fMRI functional connectivity data comprises oscillating neural signals between the auditory cortex and the rest of the brain.

12. The method of claim 1, wherein assessing the MEGI functional connectivity comprises assessing the hyposynchrony in the frontal cortex of the brain.

13. The method of claim 1, wherein assessing the hyposynchrony in the frontal cortex of the brain comprises assessing the global connectivity of the frontal cortex of the brain with the rest of the brain.

14. The method of claim 1, wherein the frontal cortex hyposynchrony magnitude is correlated with Tinnitus severity level.

15. The method of claim 1, wherein assessing the MEGI functional connectivity comprises assessing shifts in MEGI bandwidth frequencies in the frontal cortex as associated with the one or more pathology profiles of Tinnitus.

16. The method of claim 1, wherein decreased MEGI functional connectivity comprises decreased MEGI alpha-band activity ranging from 8-12 Hz.

17. The method of claim 1, wherein assessing the fMRI functional connectivity comprises assessing coherence between:
a) the caudate nucleus and the auditory cortex;
b) the caudate nucleus and the frontal lobe; or
c) a combination thereof.

18. The method of claim 1, wherein assessing the fMRI functional connectivity comprises assessing hypoconnectivity between the caudate nucleus and the frontal lobe.

19. The method of claim 1, wherein assessing the fMRI functional connectivity comprises assessing hyperconnectivity between the caudate nucleus and the frontal lobe.

20. The method of claim 1, wherein the one or more pathology profiles of Tinnitus is further associated with:
a) modulated functional connectivity between the caudate nucleus and the cuneus region of the brain;
b) modulated functional connectivity between the caudate nucleus and the superior lateral occipital cortex (sLOC); or
c) modulated functional connectivity between the caudate nucleus and the anterior supramarginal gyrus (aSMG).

21. The method of claim 1, wherein the modulated functional connectivity comprises increased functional connectivity between the caudate nucleus and the cuneus region of the brain.

22. The method of claim 1, wherein modulated functional connectivity comprises increased functional connectivity between the caudate nucleus and the sLOC.

23. The method of claim 1, wherein modulated functional connectivity comprises increased functional connectivity between the caudate nucleus and the aSMG.

24. The method of claim 9, wherein AEFs are evoked by the pure-tone stimulus at 1 kHz.

25. The method of claim 1, the method further comprises acquiring a plurality of high-resolution MR images.

26. The method of claim 25, wherein the plurality of high-resolution MR images is reconstructed into three-dimensional images.

27. The method of claim 1, wherein the acquiring comprising acquiring the MEGI functional connectivity data with a resting-state MEGI imaging device (MEGI) with the subject's eyes closed.

28. The method of claim 24, wherein the recording comprises collecting the AEF peaks with the MEGI device with the subject's eyes open.

29. The method of claim 1, wherein the acquiring comprises acquiring the MEGI functional connectivity data with the subject's eyes closed.

30.-73. (canceled)
